(19) United States
(12) Patent Application Publication
(10) Pub. No.: US 2025/0339566 A1
(43) Pub. Date: Nov. 6, 2025

(54) INHIBITORS OF FIBROBLAST ACTIVATION PROTEIN

(71) Applicant: UNIVERSITEIT ANTWERPEN, Antwerpen (BE)

(72) Inventors: Koen AUGUSTYNS, Wilrijk (BE); Lucas BEROSKE, Wilrijk (BE); Lorenzo CIANNI, Wilrijk (BE); Ingrid DE MEESTER, Wilrijk (BE); Filipe ELVAS, Wilrijk (BE); Nicolò FILIPPI, Wilrijk (BE); Sergei GRINTSEVICH, Wilrijk (BE); Muhammet TANÇ, Wilrijk (BE); Yentl VAN RYMENANT, Wilrijk (BE); Sigrid STROOBANTS, Wilrijk (BE); Pieter VAN DER VEKEN, Wilrijk (BE)

(21) Appl. No.: 18/871,115

(22) PCT Filed: Jun. 2, 2023
(86) PCT No.: PCT/EP2023/064880

§ 371 Date: Dec. 2, 2024

(30) Foreign Application Priority Data
Jun. 3, 2022 (EP) 22177276.7

(51) Int. Cl. A61K 51/04; A61K 31/4709; A61K 31/675; C07D 401/12; C07F 9/6558

(52) U.S. Cl. CPC A61K 51/0455; A61K 31/4709; A61K 31/675; C07D 401/12; C07F 9/65583

(57) ABSTRACT

The current invention relates to a compound of Formula (I) or a stereoisomer, tautomer, racemic, metabolite, prodrug, salt, hydrate, or solvate thereof, The invention also relates to a pharmaceutical composition comprising said compound. The invention also relates to said pharmaceutical composition or said compound for use as a medicine, for use in the prevention and/or treatment of diseases.

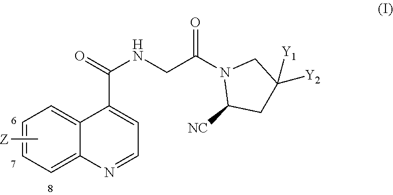

The present invention relates to a compound of Formula I or a stereoisomer, tautomer, racemic, metabolite, prodrug, salt, hydrate, or solvate thereof. The present invention further relates to a pharmaceutical composition and use thereof.

### BACKGROUND

Fibroblast activation protein (FAP), also referred to as FAPa, Seprase or a2-antiplasmin converting enzyme, is a type II integral membrane serine protease that belongs to the prolyl oligopeptidase family S9, which also includes DPPIV, DPP8, DPP9, and PREP enzymes. This family is characterized for having an exo-dipeptidyl peptidase (DPP) activity. The family is characterized by the ability to cleave a post proline bond. DPPIV, DPP8 and 9 and FAP have exopeptidase activity releasing dipeptides from peptides having a proline on the second place. PREP has an endopeptidase activity and FAP has both endo-end exopeptidase activity. FAP is mainly found as a cell surface homodimer but it has also been reported to form heterodimers with DPPIV in vivo. Purported physiological substrates of FAP endopeptidase activity include a2-antiplasmin, type I collagen, gelatin, and Fibroblast growth factor 21 (FGF21), and for the exopeptidase activity include Neuropeptide Y, B-type natriuretic peptide, substance P and peptide YY.

FAP has been implicated in pathological processes involving proliferation, tissue remodeling, chronic inflammation and/or fibrosis, including but not limited to fibrotic disease, wound healing, keloid formation, osteoarthritis, rheumatoid arthritis, and related disorders involving cartilage degradation, atherosclerotic disease, and Crohn's disease. Based on FAP's role in (patho-)physiology, documented extensively in literature, it is reasonable to foresee further and/or potential applications of inhibitors in disease domains characterized by: (a) invasion, metastasis and proliferation (including but not limited to cancer) (b) tissue remodeling and/or chronic inflammation (including but not limited to fibrotic disease, wound healing, keloid formation, osteoarthritis, rheumatoid arthritis and related disorders involving cartilage degradation) and (c) endocrinological disorders (including but not limited to disorders of glucose metabolism).

One of the most potent and selective FAP inhibitors in the public domain, is UAMC1110 (also referred to as ‘cmpd. 60’ in the scientific literature). This is an orally bioavailable molecule with a promising biopharmaceutical profile that was reported and patented by inventors of this application (see, e.g., Jansen et al. J. Med. Chem. 2014, 3053-3074 and WO2013107820). During the past years, chemical derivatives of UAMC1110 have been published with specific functionalities (e.g. radionuclides, drugs, fluorophores). All these derivatives rely on UAMC1110 for efficient and selective delivery of the functionality to FAP-expressing cells or tissues (e.g. tumors).

WO2020132661 discloses compounds for modulating fibroblast activation protein. Some of the described compounds comprise a UAMC1110 derivative in which the quinoline moiety is substituted with a phenyl or pyridine linker.

Comparably, WO2013107820 discloses inhibitors having selectivity and specificity for FAP (fibroblast activation protein). Some of the described compounds comprise a quinoline ring substituted with a halo or methoxy.

There is a need to tailor the properties of FAP inhibitors to improve safety and efficacy in vivo for different application types (e.g. therapeutic applications, PET-diagnostics, etc.). Furthermore, good stability and an ability to penetrate different kinds of tissues is desired. The current invention aims to provide a solution thereto.

### SUMMARY OF THE INVENTION

The present invention relates in a first aspect to a compound of Formula I or a stereoisomer, tautomer, racemic, metabolite, prodrug, salt, hydrate, or solvate thereof, according to claim 1. The compound comprises a linker with a quaternary ammonium cation. Compounds with a quaternary ammonium cation showed a better in vivo pharmacokinetic profile, were less susceptible to metabolization and often had an exquisite, unprecedented selectivity with respect to PREP, a protease that is very closely related to FAP. Several advantages can be connected with the greater metabolic stability, for example, increased in vivo half-life, or reduced dosage requirements, or in the framework of molecular imaging tracers higher image quality. In addition, the polarity imparted by the quaternary ammonium group promotes higher water solubility and urinary excretion, as opposed to more lipophilic linker systems that typically cause lower water solubility and can promote hepatobiliary secretion, followed by excretion via the gut. Hepatobiliary secretion can be an undesirable feature, for example in the framework of radionuclide imaging and radionuclide therapy. More specifically, important gut excretion causes a strong background signal in diagnostic imaging applications. Likewise, it can impose a higher radio-toxicological burden on the patient in radiotherapeutic applications. Noteworthy, however, the polarity imparted by a quaternary ammonium cation is still less pronounced than the polarity imparted by protonated primary, secondary and tertiary amines. In primary, secondary and tertiary amines, the electrostatic charge is typically less shielded from the environment because of the lower number of N-substituents, translating in a higher polar surface area. Moreover, the higher number of (alkyl-type) N-substituents in quaternary ammonium compounds further decreases the polarity because of the inductive stabilization of the +-charge by the substituents. This can imply that quaternary ammonium groups, despite being overall polar functionalities, can still have significantly better tissue permeability than the corresponding, protonated primary, secondary and tertiary amines. A higher permeability can be especially important in applications where dense tissue types need to be targeted by the molecules, e.g., in fibrotic tissue, atherosclerosis capping tissue or in specific tumor types with extensive desmoplasia (e.g., pancreatic cancer). The compounds as described herein were optimized to have better pharmacokinetic properties compared to overall comparable compounds that lack a quaternary ammonium group (vide infra). Furthermore, the quaternary ammonium group in the compounds according to this invention, is typically part of a short linker moiety of relatively low molecular weight. Furthermore, radio-isotopes that are eventually present in these compounds, typically are covalently attached to the linker. This is fundamentally different from many UAMC1110-derived FAP targeting compounds that are known in the state of the art. These contain one or several primary, secondary or tertiary amine functions that are protonated at physiological pH and that are part of large linker systems that also comprise a chelator for non-covalent complexation of a radionuclide. These linkers often have a molecular weight above 500 Da. This high molecular weight and the additional electrostatic charges that are present in the chelators of these frequently used compounds (e.g., DOTA, NOTA or DATA) can reasonably be expected to cause more limited tissue permeability compared to the quaternary ammonium-based linkers in this invention.

In a second aspect, the present invention relates to a pharmaceutical composition according to claim 10.

In a third aspect, the present invention relates to a use according to claim 11, claim 12, claim 13, claim 14 and claim 15. The compounds according to the invention are suited for diagnostics and/or therapeutics (including theranostics), preferably of FAP related disorders. More in particular, said compounds can be used for imaging such as PET, radiological diagnosis, preferably in situations wherein (tumor) cells express fibroblast activation protein (FAP).

### DESCRIPTION OF FIGURES

FIG. 1 shows a comparison of Compound 1 with a compound without quaternary ammonium cation (Compound 0) regarding their selectivity for FAP.

FIG. 2 shows a comparison of Compound 1 with a compound without quaternary ammonium cation (Compound 0) regarding their metabolic stability.

FIG. 3 shows a comparison of Compound 1 with a compound without quaternary ammonium cation (Compound 0) regarding their biodistribution profile.

FIG. 4 shows the in vivo uptake of [18F]UAMC-4522 (black) and [68Ga]Ga-DOTA-FAPI-04 (grey) in % ID/ml in tumor tissue over the course of 60 minutes post-injection with (dotted lines) or without (straight lines) a pre-injection of UAMC-1110.

### DETAILED DESCRIPTION OF THE INVENTION

Described herein are compounds according to Formula I:

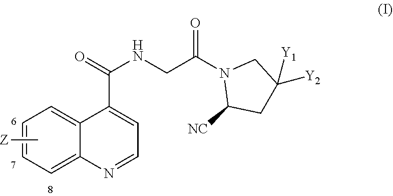

or a stereoisomer, tautomer, racemic, metabolite, prodrug, salt, hydrate, or solvate thereof, wherein Z comprises a quaternary ammonium cation.

During research on UAMC1110 derivatives, the inventors have found that the nature of the linker part in these molecules is a critical determinant of the biological profile (e.g., pharmacokinetics-profile and target selectivity) of these compounds. Combining 1) a UAMC1110 moiety or a structurally related FAP inhibiting moiety and 2) a quaternary ammonium-containing linker, can result in compounds with a more desirable pharmacokinetic profile and better target selectivity than observed for comparable molecules lacking a quaternary ammonium-based linker. This finding can be exploited to obtain new FAP inhibitors with optimized or tailored in vivo behavior. Comparably, it can be exploited to obtain new, functionally labeled (e.g., radiolabeled or drug-labeled) UAMC1110-derivatives with a more desirable pharmacokinetic profile and/or target selectivity.

To the best of our knowledge, the optimization or tailoring of UAMC1110 derivatives via the introduction of specific linker moieties has not been widely explored.

The compounds can be used for inhibiting fibroblast activation protein (FAP). In certain embodiments, the compound is used to treat a disease or a disorder mediated by FAP in an individual. Such diseases or disorders can include or be characterized by proliferation, tissue remodeling, chronic inflammation, obesity, glucose intolerance, and/or insulin insensitivity. In some embodiments, the compound is used to diagnose and/or treat diseases characterized by proliferation, tissue remodeling, chronic inflammation, obesity, glucose intolerance, and/or insulin insensitivity. A non-limiting list of such diseases includes cancer, fibrosis or diseases characterized by fibrotic lesions, atherosclerosis, arthritis and diabetes.

### Definitions

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

The recitation of numerical ranges by endpoints includes all numbers and fractions subsumed within that range, as well as the recited endpoints.

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a C₁-C₂₀ alkyl”), having 1 to 10 carbon atoms (a C₁-C₁₀ alkyl), having 6 to 10 carbon atoms (a C₆-C₁₀ alkyl), or having 1 to 4 carbon atoms (a C₁-C₄ alkyl). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Halo” or “halogen” refers to elements of the Group 17 series having atomic number 9 to 85. Preferred halo groups include the radicals of fluorine, chlorine, astatine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl.

A “heterocycle” or “heterocyclic” as used herein refers to a saturated or an unsaturated non-aromatic cyclic group having a single ring or multiple condensed rings and having from 1 to 14 annular carbon atoms and from 1 to 6 annular heteroatoms, such as nitrogen, phosphorous, sulfur or oxygen, and the like. A heterocycle comprising more than one ring may be fused, bridged or spiro, or any combination thereof, but excludes heteroaryl groups. The heterocyclic group may be optionally substituted independently with one or more substituents described herein. Particular heterocyclic groups are 3 to 14-membered rings having 1 to 13 annular carbon atoms and 1 to 6 annular heteroatoms independently selected from nitrogen, phosphorous, oxygen and sulfur, 3 to 12-membered rings having 1 to 11 annular carbon atoms and 1 to 6 annular heteroatoms independently selected from nitrogen, phosphorous, oxygen and sulfur, 3 to 10-membered rings having 1 to 9 annular carbon atoms and 1 to 4 annular heteroatoms independently selected from nitrogen, phosphorous, oxygen and sulfur, 3 to 8-membered rings having 1 to 7 annular carbon atoms and 1 to 4 annular heteroatoms independently selected from nitrogen, phosphorous, oxygen and sulfur, or 3 to 6-nienibered rings having 1 to 5 annular carbon atoms and 1 to 4 annular heteroatoms independently selected from nitrogen, phosphorous, oxygen and sulfur. Particular heterocyclic groups are monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, phosphorous, oxygen and sulfur. Particular heterocyclic groups are polycyclic non-aromatic rings having from 1 to 12 annular carbon atoms and 1 to 6 annular heteroatoms independently selected from nitrogen, phosphorous, oxygen and sulfur.

The term “quaternary ammonium cation” used herein is intended to refer to a cation containing at least one nitrogen atom carrying a positive electric charge, which nitrogen atom is bonded only to carbon. The positive electrostatic charge is present, independently of the pH. The nitrogen atom may be saturated, being bonded to four carbon atoms by single bonds, or may be unsaturated, being bonded to two carbon atoms by single bonds and to a third carbon atom by a double bond. Where the nitrogen atom is unsaturated, it may be part of a heteroaromatic ring, such as an imidazolium cation. Where the nitrogen atom is saturated, it may be part of an alicyclic ring, such as a pyrrolidinium or a piperidinium cation.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

As used herein, “treatment” or “treating” is an approach for obtaining beneficial or desired results including clinical results. Beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more oilier medications required to treat the disease, enhancing effect of another medication, delaying the progression of the disease, increasing the quality of life, and/or prolonging survival. The methods described herein contemplate any one or more of these aspects of treatment.

The term “diagnostic” as used herein means having the ability to detect, monitor, follow, and/or identify a disease or condition in an animal (including humans) or from a biological sample.

The term “theragnostic” as used herein means having the combined effects of a therapeutic and a diagnostic composition. The composition is suitable to identify (diagnose) and to deliver therapy (therapeutics).

As used herein, the term “radionuclide” includes metallic and non-metallic radionuclides. The radionuclide is chosen based on the medical application of the radiolabeled pharmaceutical composition. When the radionuclide is a metallic radionuclide, a chelator is typically employed to bind the metallic radionuclide to the rest of the molecule. When the radionuclide is a non-metallic radionuclide, the non-metallic radionuclide is typically linked directly to the rest of the molecule. Radionuclides are routinely used in nuclear medicine for the diagnosis and/or therapy of various diseases. The radiolabeled pharmaceutical agent, for example, a radiolabeled medicament, contains a radionuclide which serves as the radiation source. Radionuclide therapy is a therapy using said radionuclides.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration and/or have been approved by the administrations such as EMA and/or United States Food and Drug Administration as being acceptable for use in humans or domestic animals.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc; coatings include, e.g, cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for tablets include, e.g., dextrose, fructose de, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

### Compounds

In a first aspect, the invention relates to a compound of Formula I.

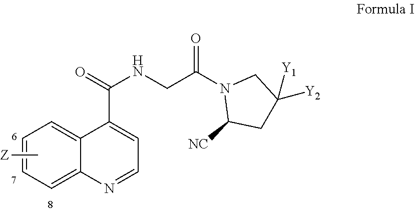

In an embodiment, the compound of Formula I is present as a stereoisomer, tautomer, racemic, metabolite, prodrug, salt, hydrate, or solvate thereof. In an embodiment, the compound of Formula I is present as a pharmaceutically acceptable salt, stereoisomer or tautomer thereof. The inventors have found that compounds according to Formula I are very effective in inhibiting the fibroblast activation protein (FAP).

In an embodiment Y₁ and Y₂ are independently H or F, preferably Y₁ and Y₂ are both F. In a further embodiment F is present in natural proportions of atomic isotopes. The presence of said F results in improved selectivity characteristics when compared to other FAP inhibitors, while retaining high affinity for the target enzyme. In another embodiment, Y₁ and Y₂ are both H. In another embodiment, Y₁ is F and Y₂ is H. In another embodiment, Y₁ and Y₂ are both ¹⁸F.

In an embodiment, linker (Z) comprises an oxygen, wherein said oxygen is covalently bound to the quinoline structure of said compound on position 6, 7 or 8. Positions of the quinoline structures are numbered as shown in Formula II (see below):

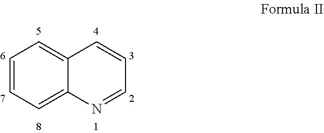

In an embodiment the linker is covalently linked via oxygen to the quinoline structure of said compound on position 6. In an embodiment the linker is covalently linked via oxygen to the quinoline structure of said compound on position 7. In an embodiment the linker is covalently linked via oxygen to the quinoline structure of said compound on position 8.

In an embodiment said linker comprises a quaternary ammonium cation. In a further embodiment, the quaternary ammonium cation is bound to 4 carbon atoms. Compounds with a quaternary ammonium cation showed a better in vivo pharmacokinetic profile, were less susceptible to metabolization and had an exquisite, unprecedented selectivity with respect to PREP, a protease that is very closely related to FAP. All these properties are advantageous in certain therapeutic, diagnostic or theragnostic settings.

In an embodiment said linker, optionally comprising a radionuclide, has a molecular weight of maximal 1000 Da, preferably maximal 750 Da, preferably maximal 600 Da, more preferably maximal 500 Da, more preferably maximal 400 Da, even more preferably maximal 300 Da and most preferably maximal 200 Da. In an embodiment said linker, optionally comprising a radionuclide, has a molecular weight of maximal 1000 Da, preferably maximal 750 Da, preferably maximal 600 Da, more preferably maximal 500 Da, more preferably maximal 400 Da and a molecular weight of at least 100 Da, preferably at least 120 Da, more preferably more than 150 Da. In an embodiment, the linker, without the substituted radionuclide, has a molecular weight of maximal 600 Da, more preferably maximal 500 Da, more preferably maximal 400 Da, even more preferably maximal 300 Da and most preferably maximal 200 Da. It was shown that compounds having a linker with a molecular weight that is higher than the above-mentioned threshold have a negative impact on the pharmacokinetics of the compound. In vivo stability, i.e., biochemical resistance in blood serum under physiological conditions, is essential to function efficiently. Large chelating linkers can relatively easily be disturbed or degraded, and their functionality might be reduced.

In the descriptions herein, it is understood that every description, embodiment or aspect of a moiety may be combined with every description, embodiment or aspect of other moieties the same as if each and every combination of descriptions is specifically and individually listed. For example, every description, embodiment or aspect provided herein with respect to Z of formula (I) may be combined with every description, embodiment or aspect of Y¹ and/or Y² the same as if each and every combination were specifically and individually listed. It is also understood that all descriptions, embodiments or aspects of formula (I), where applicable, apply equally to other formulae detailed herein, and are equally described, the same as if each and every description, embodiment or aspect were separately and individually listed for all formulae.

In an embodiment, said linker is linked to a radionuclide. In an embodiment, the radionuclide is chosen from the group of ¹⁸F, ¹²⁰I, ¹²²I, ¹²³I, ¹²⁴I, ¹²⁵I, ¹³¹I, ²¹¹At, ⁴³Sc, ⁴⁴Sc, ⁵¹Mn, ⁵²Mn, ⁶⁴Cu, ⁶⁷Ga, ⁶⁸Ga, ⁸⁶Y, ⁸⁹Zr, ¹¹In, ¹⁵²Tb, ¹⁵⁵Tb, ²⁰³Pb, ⁷⁶Br, ⁷⁷Br, ⁴⁷Sc, ⁶⁷Cu, ⁸⁹Sr, ⁹⁰Y, ¹⁵³Sm, ¹⁴⁹Tb, ¹⁶¹Tb, ¹⁷⁷Lu, ¹⁸⁶Re, ¹⁸⁸Re, ²¹²Pb, ²¹³Bi, ²²³Ra, ²²⁵Ac, ²²⁶Th, ²²⁷Th ²²⁵Ac, ²¹²Bi, ²¹³Bi, and ¹⁷⁷Lu. In an embodiment, said radionuclide is selected from ¹²⁰I, ¹²²I, ¹²³I, ¹²⁴I, ¹²⁵I, ¹³¹I or ²¹¹At. In an embodiment, the linker is bound to the radioisotope covalently. In an embodiment, the radionuclide has a half-life of 10 minutes to 60 days, preferably 1 hour to 7 days, more preferably 2 hours to 3 days.

In an embodiment, the radionuclide is covalently bound to the linker. A covalent bond between the linker and the radionuclide is considered more stable compared to complexed radionuclide in a large chelating structure.

In an embodiment, a counterion is present to offset the positive charge of the quaternary ammonium cation. In a further embodiment, said counterion is selected from the group of: halide, hydroxide, carboxylate, sulphate, phosphate, nitrate, alkyl sulfonate, aryl sulfonate, other organic anions and combinations thereof. In a further embodiment, the counterion is a monovalent anion. In a further embodiment, the counterion is Cl or Br or a combination thereof.

In the descriptions herein, it is understood that the wavy line represents the point of attachment to the rest of the compound. If a structure is not symmetrical, the wavy line closer to the quinoline structure shown in Formula I, is accompanied of a *. If a structure is not symmetrical, the wavy line closer to E, is accompanied of a ●.

In an embodiment, —Z is —O-L₁-A-(L₂-D)₀₋₂-L₃-E, wherein:
each L₁, L₂, L₃ is independently

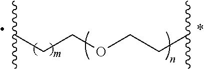

wherein n is 0, 1, 2 or 3 and m is 0, 1, 2, 3, or 4;
A is selected from the group of:

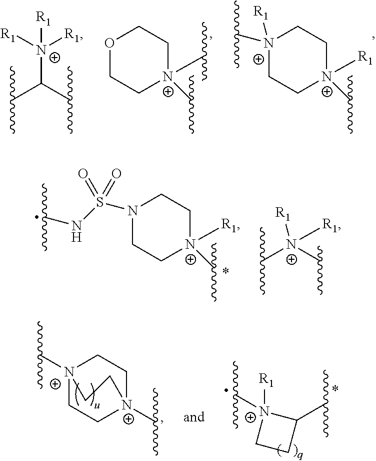

wherein q is 0, 1, 2 or 3 and u is 0, 1, 2 or 3;
D is selected from the group of:

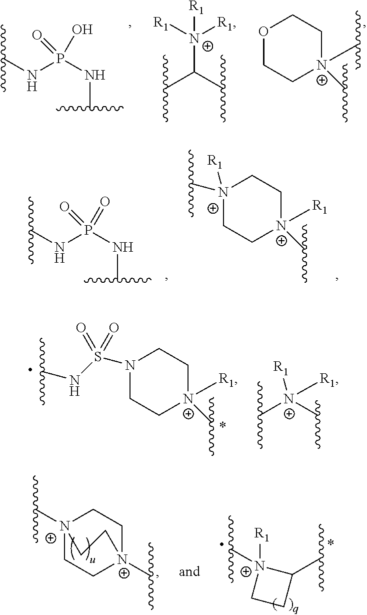

wherein q is 0, 1, 2 or 3 and u is 0, 1, 2 or 3; and
E is selected from the group of:
R₁, a chelating moiety, a benzamide, a borane, a carborane, a metal complex and a 5-, 6- or 7-membered aromatic ring, optionally heterocyclic, optionally substituted;
and wherein each R₁ independently is selected from the group of:
H, C₁₋₄ alkyl and C₁₋₄ alkyl substituted with halo, preferably ¹⁸F, ²¹¹At, ¹²⁰I, ¹²²I, ¹²³I, ¹²⁴I, ¹²⁵I or ¹³¹I. In an embodiment, R₁ is an alkyl comprising 2, 3 or 4 radionuclides. In an embodiment, R₁ comprises deuterium (d) and ¹⁸F, preferably R₁ is fluoro-¹⁸F methyl-d2.

In an embodiment, E is a chelating moiety, wherein the chelating moiety is a radical selected from the group of: DOTA (1,4,7,10-tetraazacyclododecane-1,4,7,10-tetraacetic acid), CB-DO2A (4,10-bis(carboxymethyl)-1,4,7,10-tetraazabicyclo[5.5.2]tetradecane), TCMC (1,4,7,10-tetrakis(carbamoylmethyl)-1,4,7,10-tetraazacyclododecane), 3p-C-DEPA (2-[(carboxymethyl)]-[5-(4-nitrophenyl-1-[4,7,10-tris-(carboxymethyl)-1,4,7,10-tetraazacyclododecan-1-yl]pentan-2-yl)-amino]acetic acid), TETA (1,4,8,11-tetraazacyclotetradecane-1,4,8,11-tetraacetic acid), NOTA (1,4,7-triazacyclononane-1,4,7-triacetic acid), NETA ({4-[2-(bis-carboxymethylamino)-ethyl]-7-carboxymethyl-[1,4,7]triazonan-1-yl}-acetic acid), 3p-C-NEPA (2-{[2-(4-{2-[Bis(carboxymethyl)amino]-5-(4-nitrophenyl)pentyl}-7-(carboxymethyl)-1,4,7-triazonan-1-yl)ethyl](carboxymethyl) amino}acetic acid), 3p-C-NETA-NCS ({4-[2-(Bis-carboxymethylamino)-5-(4-isothiocyanatophenyl) pentyl]-7-carboxymethyl-[1,4,7]triazonan-1-yl}acetic acid), TACN-TM (N,N′,N″, tris(2-mercaptoethyl)-1,4,7-triazacyclononane), DTPA (diethylenetriaminepentaacetic acid), CHX-A″-DTPA (2-(p-isothiocyanatobenzyl)-cyclohexyldiethylenetriaminepentaacetic acid), TRAP (1,4,7-triazacyclononane-1,4,7-tris[methyl(2-carboxyethyl)phosphinic acid]), H2dedpa (1,2-[[6-(carboxy)-pyridin-2-yl]-methylamino]ethane), H4octapa (N,N′-bis(6-carboxy-2-pyridylmethyl)-ethylenediamine-N,N′-diacetic acid), H2azapa (N,N′-[1-benzyl-1,2,3-triazole-4-yl]methyl-N,N′-[6-(carboxy)pyridin-2-yl]-1,2-diaminoethane), H5decapa (N,N″-[[6-(carboxy)pyridin-2-yl]methyl]-diethylenetriamine-N,N′,N″-triacetic acid), HBED (N,N′-bis(2-hydroxybenzyl)-ethylenediamine-N,N′-diacetic acid), SHBED (N,N′-bis(2-hydroxy-5-sulfobenzyl)-ethylenediamine-N,N′-diacetic acid), PCTA (3,6,9,15-tetraazabicyclo[9.3.1]-pentadeca-1(15),11,13-triene-3,6,9,-triacetic acid), HEHA (1,4,7,10,13,16-hexaazacyclohexadecane-N,N′,N″,N′″,N″″,N′″″-hexaacetic acid), and PEPA (1,4,7,10,13-pentaazacyclopentadecane-N,N′,N″,N′″,N″″,N′″″-pentaacetic acid). In a further embodiment, a radionuclide is bound to the chelating moiety in a stable coordination complex. In a further embodiment, the radionuclide is suitable for single photon emission computed tomography (SPECT, e.g. ⁶⁷Ga, ^{99m}Tc, ¹¹¹In, ¹⁷⁷Lu), or positron emission tomography (PET, e.g. ⁶⁸Ga, ⁶⁴Cu, ⁴⁴Sc, ⁸⁶Y, ⁸⁹Zr), or therapeutic applications (e.g. ⁴⁷Sc, ^{114m}In, ¹⁷⁷Lu, ⁹⁰Y, ^{212/213}Bi, ²¹²Pb, ²²⁵Ac, ^{186/188}Re).

In an embodiment, E is a benzamide, wherein said benzamide is selected from the group of:

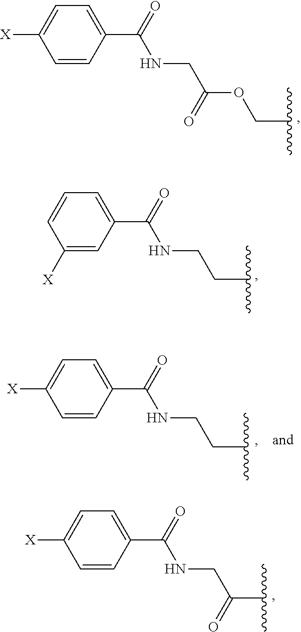

wherein X is chosen from the group of SnBu₃, I, ¹²⁵, ²¹¹At, halo, ¹⁸F and ²H.

In an embodiment, E is a borane or a carborane, wherein said borane or carborane is selected from the group of:
decaborate optionally substituted with X, dodecaborate optionally substituted with X,

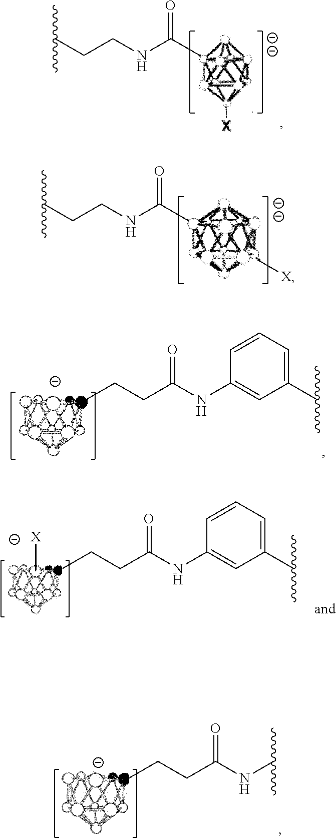

wherein each X is selected from the group of H, ²H, I, ¹²⁵I and ²¹¹At; the open circles represent B (where substituted) or BH atoms; the filled circles represent carbon atoms. In an embodiment, a counterion is present, wherein said counterion is a cation, preferably selected from the group of Bu₄N⁺ and Et₃NH⁺.

In an embodiment, E is a metal complex, wherein said metal is a metal from the platinum group. In an embodiment, E is a metal complex, wherein said metal complex is selected from the group of:

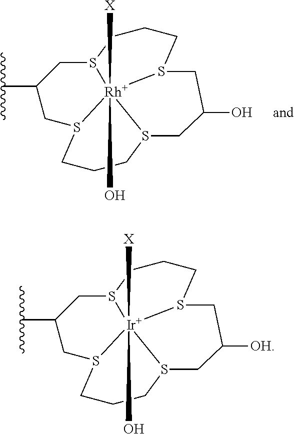

Several classes can be identified in the structures according to the current invention. A first class comprises compounds according to Formula I or a stereoisomer, tautomer, racemic, metabolite, prodrug, salt, hydrate, or solvate thereof,
wherein Z is selected from the group of

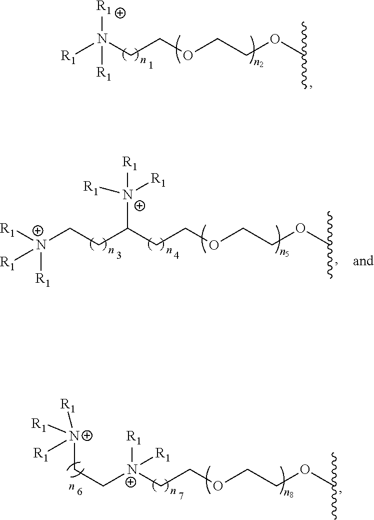

wherein each R₁ is independently selected from the group of:
—H, —CH₃, CH₂CH₃, —CH₂CH₂CH₃, —CH₂CH₂CH₂CH₃, —CH₂F, CH₂CH₂F, —CH₂CH₂CH₂F, —CH₂CH₂CH₂CH₂F, —CH₂I, CH₂CH₂I, —CH₂CH₂CH₂I, —CH₂CH₂CH₂CH₂At, —CH₂At, CH₂CH₂At, —CH₂CH₂CH₂At, and —CH₂CH₂CH₂CH₂At wherein n₁, n₂, n₃, n₄, n₅, n₆, n₇, n₈ is independently 0-4.

In an embodiment, F is present as ¹⁸F. In an embodiment, I is present as ¹²⁰I, ¹²²I, ¹²³I, ¹²⁴I, ¹²⁵I or ¹³¹I. In an embodiment, At is present as ¹⁹¹At, ¹⁹³At, ¹⁹⁴At, ¹⁹⁵At, ¹⁹⁶At, ¹⁹⁷At, ¹⁹⁸At, ¹⁹⁹At, ²⁰⁰At, ²⁰¹At, ²⁰²At, ²⁰³At, ²⁰⁴At, ²⁰⁵At, ²⁰⁶At, ²⁰⁷At, ²⁰⁸At, ²⁰⁹At, ²¹⁰At, ²¹¹At, ²¹²At, ²¹³At, ²¹⁴At, ²¹⁵At, ²¹⁶At, ²¹⁷At, ²¹⁸At, ²¹⁹At, ²²⁰At, ²²¹At, ²²²At, ²²³At, more particularly ²¹¹At. In an embodiment one H is present as ²H or ³H. In an embodiment, C is present as ¹³C, ¹¹C or ¹⁴C. In an embodiment, N is present as ¹³N. In an embodiment, O is present as ¹⁵O or ¹⁷O.

In said compounds of this first class, every n is independently selected from 0-4. For 0-4, a selection can be made between 0, 1, 2, 3 and 4. In an embodiment, each n is independently selected from 0-3. In an embodiment, n is selected from 0 or 1. In an embodiment, for all n in the compound n is 0. In an embodiment, for all n in the compound n is 1.

When selecting moieties for R¹, one should be aware of the requirements that the compound should contain a quaternary ammonium cation. A R₁ bound to a first nitrogen can only be hydrogen if there is a second nitrogen wherein the second nitrogen is a cation and is bound to only carbon and or phosphorous atoms.

Preferably each R₁ independently is selected from the group of:
—CH₃, CH₂CH₃, —CH₂CH₂CH₃, —CH₂CH₂CH₂CH₃, —CH₂F, CH₂CH₂F, —CH₂CH₂CH₂F, —CH₂CH₂CH₂CH₂F, —CH₂I, CH₂CH₂I, —CH₂CH₂CH₂I, —CH₂CH₂CH₂CH₂At, —CH₂I, CH₂CH₂At, —CH₂CH₂CH₂At, and —CH₂CH₂CH₂CH₂At wherein F is present as ¹⁸F and/or I as ¹²⁰I, ¹²²I, ¹²³I, ¹²⁴I, ¹²⁵I or ¹³¹I and/or At as ²¹¹At.

In an embodiment, the linker comprises one F and no I. In an embodiment, the linker comprises one I and no F. In an embodiment, the linker comprises one F and one I.

Examples of compounds according to this first class include (but are not limited to):

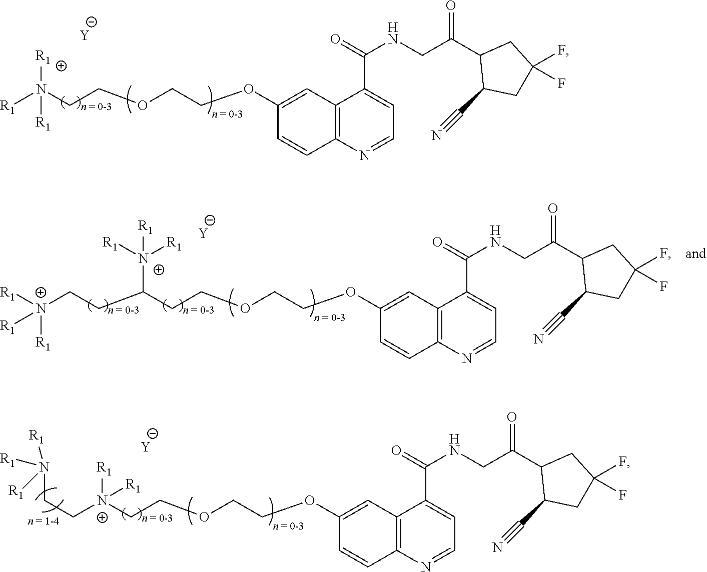

wherein Y is a counterion, preferably Cl or Br;
and wherein every n is independently selected from the range stipulated in proximity of the parenthesis. For example, for n=0-3, a selection can be made between 0, 1, 2 and 3. In an embodiment, the selected n is the lowest number suggested by the range. In an embodiment, for all n in one structure the lowest number suggested by the range is selected. In an embodiment, the selected n is the lowest number +1 suggested by the range. In an embodiment, for all n in one structure the lowest number suggested by the range +1 is selected.

A second class of compounds according to an embodiment of the current invention, comprise compounds according to Formula I or a stereoisomer, tautomer, racemic, metabolite, prodrug, salt, hydrate, or solvate thereof, wherein Z is selected from the group of

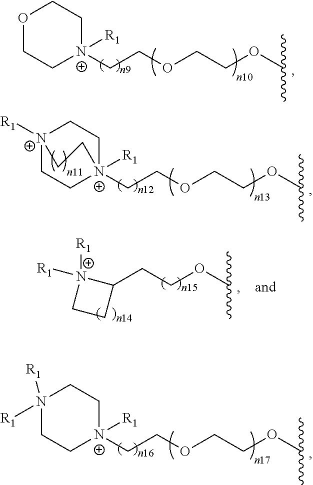

wherein each R₁ is independently selected from the group of:
—H, —CH₃, CH₂CH₃, —CH₂CH₂CH₃, —CH₂CH₂CH₂CH₃, —CH₂F, CH₂CH₂F, —CH₂CH₂CH₂F, —CH₂CH₂CH₂CH₂F, —CH₂I, CH₂CH₂I, —CH₂CH₂CH₂I, —CH₂CH₂CH₂CH₂I, —CH₂At, CH₂CH₂At, —CH₂CH₂CH₂At, —CH₂CH₂CH₂CH₂At, COOC(CH₃)₃ and COC₆H₆—R₂, wherein n₉, n₁₀, n₁₁, n₁₂, n₁₃, n₁₄, n₁₅, n₁₆, n₁₇ is independently 0-4. In a further embodiment, R₂ is selected from the group consisting of I, F, At and B(OH)₂.

In an embodiment, F is present as ¹⁸F. In an embodiment, I is present as ¹²⁰I, ¹²²I, ¹²³I, ¹²⁴I, ¹²⁵I or ¹³¹I. In an embodiment, At is present as ¹⁹¹At, ¹⁹³At, ¹⁹⁴At, ¹⁹⁵At, ¹⁹⁶At, ¹⁹⁷At, ¹⁹⁸At, ¹⁹⁹At, ²⁰⁰At, ²⁰¹At, ²⁰²At, ²⁰³At, ²⁰⁴At, ²⁰⁵At, ²⁰⁶At, ²⁰⁷At, ²⁰⁸At, ²⁰⁹At, ²¹⁰At, ²¹¹At, ²¹²At, ²¹³At, ²¹⁴At, ²¹⁵At, ²¹⁶At, ²¹⁷At, ²¹⁸At, ²¹⁹At, ²²⁰At, ²²¹At, ²²²At, ²²³At, more particularly ²¹¹At. In an embodiment one H is present as ²H or ³H. In an embodiment, C is present as ¹³C, ¹¹C or ¹⁴C. In an embodiment, N is present as ¹³N. In an embodiment, O is present as ¹⁵O or ¹⁷O.

Every n is independently selected from 0-4. For 0-4, a selection can be made between 0, 1, 2, 3 and 4. In an embodiment, each n is independently selected from 0-3. In an embodiment, n is selected from 0 or 1. In an embodiment, for all n in the compound n is 0. In an embodiment, for all n in the compound n is 1.

When selecting moieties for R₁, one should be aware of the requirements that the compound should contain a quaternary ammonium cation. A R₁ bound to a first nitrogen can only be hydrogen if there is a second nitrogen wherein the second nitrogen is a cation and is bound to only carbon atoms.

Preferably each R₁ independently is selected from the group of:
—CH₃, CH₂CH₃, —CH₂CH₂CH₃, —CH₂CH₂CH₂CH₃, —CH₂F, CH₂CH₂F, —CH₂CH₂CH₂F, —CH₂CH₂CH₂CH₂F, —CH₂I, CH₂CH₂I, —CH₂CH₂CH₂I, —CH₂CH₂CH₂CH₂I, —CH₂At, CH₂CH₂At, —CH₂CH₂CH₂At, —CH₂CH₂CH₂CH₂At, COOC(CH₃)₃ and COC₆H₆—R₂, wherein F is present as ¹⁸F and/or I as ¹²⁰I, ¹²²I, ¹²³I, ¹²⁴I, ¹²⁵I or ¹³¹I and/or At as ²¹¹At and wherein R₂ is selected from the group consisting of I, F, At and B(OH)₂.

In an embodiment, the linker comprises one F and no I. In an embodiment, the linker comprises one I and no F.

In a second class of compounds according to an embodiment of the current invention, the compound is selected from the group of:

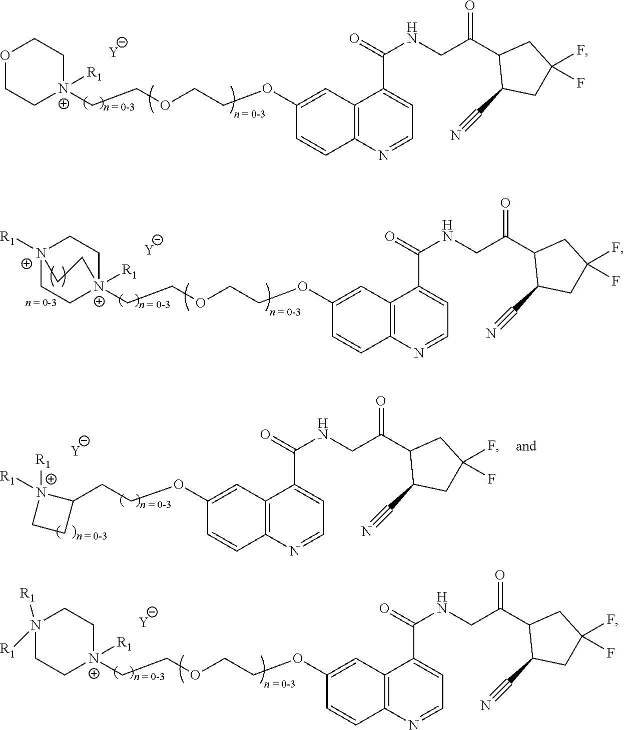

wherein Y is a counterion, preferably Cl or Br,
and wherein every n is independently selected from the range stipulated in proximity of the parenthesis. For example, for n=0-3, a selection can be made between 0, 1, 2 and 3. In an embodiment, the selected n is the lowest number suggested by the range. In an embodiment, for all n in one structure the lowest number suggested by the range is selected. In an embodiment, the selected n is the lowest number +1 suggested by the range. In an embodiment, for all n in one structure the lowest number suggested by the range +1 is selected.

In a third class of compounds according to an embodiment of the current invention, the compound is a compound according to Formula I or a stereoisomer, tautomer, racemic, metabolite, prodrug, salt, hydrate, or solvate thereof,
wherein Z is selected from the group of

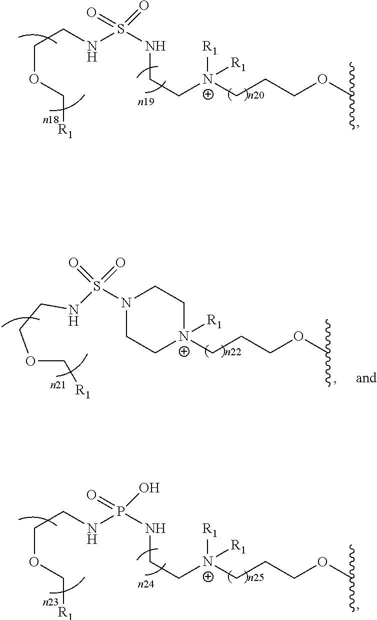

wherein each R₁ is independently selected from the group of:
—H, —CH₃, CH₂CH₃, —CH₂CH₂CH₃, —CH₂CH₂CH₂CH₃, —CH₂F, CH₂CH₂F, —CH₂CH₂CH₂F, —CH₂CH₂CH₂CH₂F, —CH₂At, CH₂CH₂At, —CH₂CH₂CH₂At, —CH₂CH₂CH₂CH₂At, —CH₂I, CH₂CH₂I, —CH₂CH₂CH₂I, and —CH₂CH₂CH₂CH₂I, and wherein n₁₈, n₁₉, n₂₀, n₂₁, n₂₂, n₂₃, n₂₄, n₂₅ is independently 0-4.

Every n is independently selected from 0-4. For 0-4, a selection can be made between 0, 1, 2, 3 and 4. In an embodiment, each n is independently selected from 0-3. In an embodiment, n is selected from 0 or 1. In an embodiment, for all n in the compound n is 0. In an embodiment, for all n in the compound n is 1.

In an embodiment, F is present as ¹⁸F. In an embodiment, F is present as ¹⁸F. In an embodiment, I is present as ¹²⁰I, ¹²²I, ¹²³I, ¹²⁴I, ¹²⁵I or ¹³¹I. In an embodiment, At is present as ²¹¹At. In an embodiment one H is present as ²H or ³H. In an embodiment, C is present as ¹³C, ¹¹C or ¹⁴C. In an embodiment, N is present as ¹³N. In an embodiment, O is present as ¹⁵O or ¹⁷O.

When selecting moieties for R¹, one should be aware of the requirements that the compound should contain a quaternary ammonium cation. A R₁ bound to a first nitrogen can only be hydrogen if there is a second nitrogen wherein the second nitrogen is a cation and is bound to only carbon atoms.

Preferably each R₁ independently is selected from the group of:
—CH₃, CH₂CH₃, —CH₂CH₂CH₃, —CH₂CH₂CH₂CH₃, —CH₂F, CH₂CH₂F, —CH₂CH₂CH₂F, —CH₂CH₂CH₂CH₂F, —CH₂At, CH₂CH₂At, —CH₂CH₂CH₂At, —CH₂CH₂CH₂CH₂At, —CH₂I, CH₂CH₂I, —CH₂CH₂CH₂I, and —CH₂CH₂CH₂CH₂I, wherein F is present as ¹⁸F and/or At is present as ²¹¹At and/or I as ¹²⁰I, ¹²²I, ¹²³I, ¹²⁴I, ¹²⁵I or ¹³¹I.

In an embodiment, the linker comprises one F and no I. In an embodiment, the linker comprises one I and no F.

In a third class of compounds according to an embodiment of the current invention, the compound is selected from the group of:

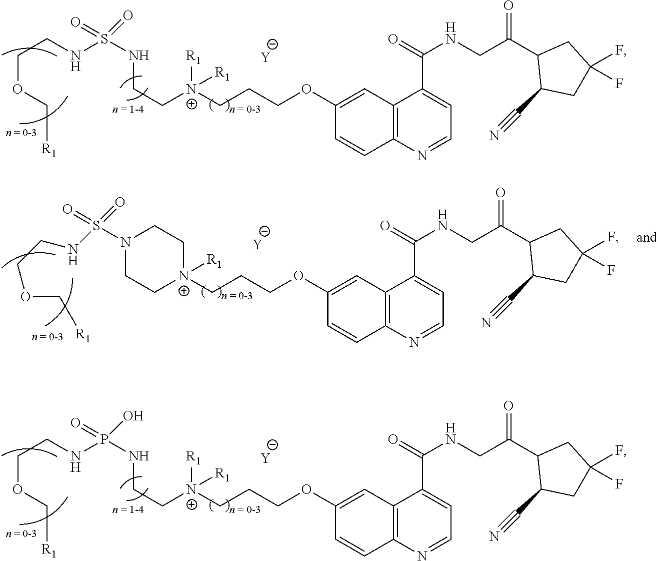

wherein Y is a counterion, preferably Cl or Br;
and wherein every n is independently selected from the range stipulated in proximity of the parenthesis. For example, for n=0-3, a selection can be made between 0, 1, 2 and 3. In an embodiment, the selected n is the lowest number suggested by the range. In an embodiment, for all n in one structure the lowest number suggested by the range is selected. In an embodiment, the selected n is the lowest number +1 suggested by the range. In an embodiment, for all n in one structure the lowest number suggested by the range +1 is selected.

In an embodiment, the compound is a compound according to Formula I or a stereoisomer, tautomer, racemic, metabolite, prodrug, salt, hydrate, or solvate thereof, wherein Z comprises an aromatic ring, optionally heterocyclic and 5-, 6- or 7-membered.

In an embodiment, the compound is a compound according to Formula I or a stereoisomer, tautomer, racemic, metabolite, prodrug, salt, hydrate, or solvate thereof, wherein Z comprises a 5-, 6- or 7-membered aromatic ring, optionally heterocyclic. In an embodiment, said 5-, 6- or 7-membered aromatic ring, optionally heterocyclic, and optionally substituted with R₂ and R₃ is shown in Formula III:

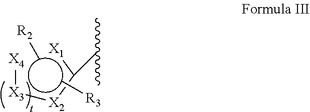

The X₁, X₂, X₃ and X₄ are independently chosen from: C, S, N and O, to form a pharmaceutically acceptable 5-, 6- or 7-membered aromatic ring which can be substituted with R₂ and R₃, preferably X₁, X₂, X₃ and X₄ are chosen from: C, N and O to form a 5-, 6- or 7-membered aromatic ring. The t is chosen from 1, 2 and 3. In an embodiment, the aromatic ring comprises one heteroatom. In an embodiment, the aromatic ring comprises two heteroatoms. In an embodiment, the aromatic ring comprises no heteroatoms. In a further embodiment, R₂ is bound to X₄ and X₄ is carbon. In an embodiment, R₃ is bound to X₂ and X₂ is carbon. In another embodiment, R₃ is bound to X₁ and X₁ is carbon.

In an embodiment, the 5-, 6- or 7-membered aromatic ring, optionally heterocyclic, is selected from the group of: furan, pyrrole, pyrazole, isoxazole, imidazole, 1,2,3-triazole, 1,2,4-triazole, oxazole, thiazole, benzene, thiophene, pyridine, pyrazine, pyrimidine, pyridazine or triazine.

In an embodiment, the 5-, 6- or 7-membered aromatic ring, optionally heterocyclic, is selected from the group of:

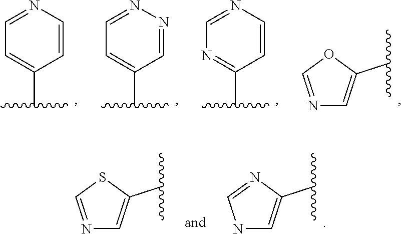

In an embodiment, R₂ is selected from the group of: ¹⁸F, ¹²⁰I, ¹²²I, ¹²³I, ¹²⁴I, ¹²⁵I, ¹³¹I and At, preferably ²¹¹At.

In an embodiment, R₃ is selected from the group of: guanidine, aminomethyl and dialkylaminomethyl. In a further embodiment, the dialkylaminomethyl is selected from the group of: dimethylaminomethyl, diethylaminomethyl and dipropylaminoethyl. Guanidine has a general structure (R₁₀R₁₁N)(R₁₂R₁₃N)C═N—R₁₄. The guanidine can be bound to the rest of the compound on any position. In an embodiment, R₁₀ is the rest of the compound and R₁₁-R₁₄ are H. In another embodiment R₁₀-R₁₃ are H and the rest of the compound is R₁₄.

In a fourth class of compounds according to an embodiment of the current invention, the compound is a compound according to Formula I or a stereoisomer, tautomer, racemic, metabolite, prodrug, salt, hydrate, or solvate thereof, wherein linker (Z) is selected from the group of:

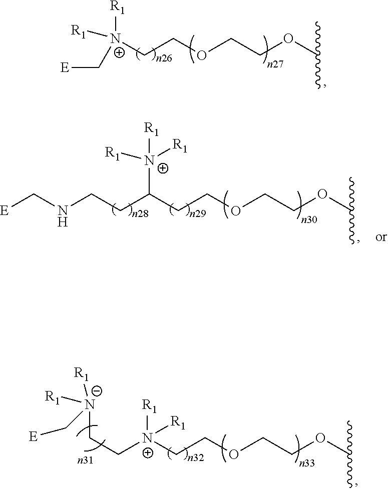

wherein
each R₁ is independently selected from the group of:
—H, —CH₃, CH₂CH₃, —CH₂CH₂CH₃, and —CH₂CH₂CH₂CH₃;
n₂₆, n₂₇, n₂₈, n₂₉, n₃₀, n₃₁, n₃₂, n₃₃ is independently 0-4;
E is

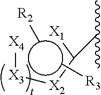

wherein each X₁, X₂, X₃, X₄ is independently selected from the group of C, O and N;
t is independently 1, 2 or 3;
each R₂ is independently selected from the group of:
¹⁸F, ¹²⁰I, ¹²²I, ¹²³I, ¹²⁴I, ¹²⁵I, ¹³¹I and ²¹¹At; and
each R₃ is independently selected from the group of:
guanidine, aminomethyl and dialkylaminomethyl.

In an further embodiment Z is selected from the group of

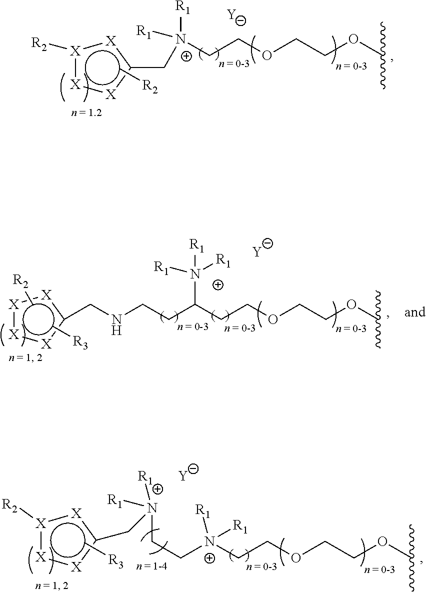

wherein
each X is independently selected from the group of C, O and N,
each R₁ is independently selected from the group of:
—H, —CH₃, CH₂CH₃, —CH₂CH₂CH₃, and —CH₂CH₂CH₂CH₃,
each R₂ is independently selected from the group of:
¹⁸F, ¹²⁰I, ¹²²I, ¹²³I, ¹²⁴I, ¹²⁵I, ¹³¹I and ²¹¹At, preferably ²¹¹At,
each R₃ is independently selected from the group of:
guanidine, aminomethyl and dialkylaminomethyl, preferably the dialkylaminomethyl is selected from the group of: dimethylaminomethyl, diethylaminomethyl and dipropylaminoethyl.

When selecting moieties for X, one should be aware of the requirements stipulated in the other embodiments of the current invention, so that a 5-, 6- or 7-membered aromatic ring is formed.

In a fourth class of compounds according to an embodiment of the current invention, the compound is selected from the group of:

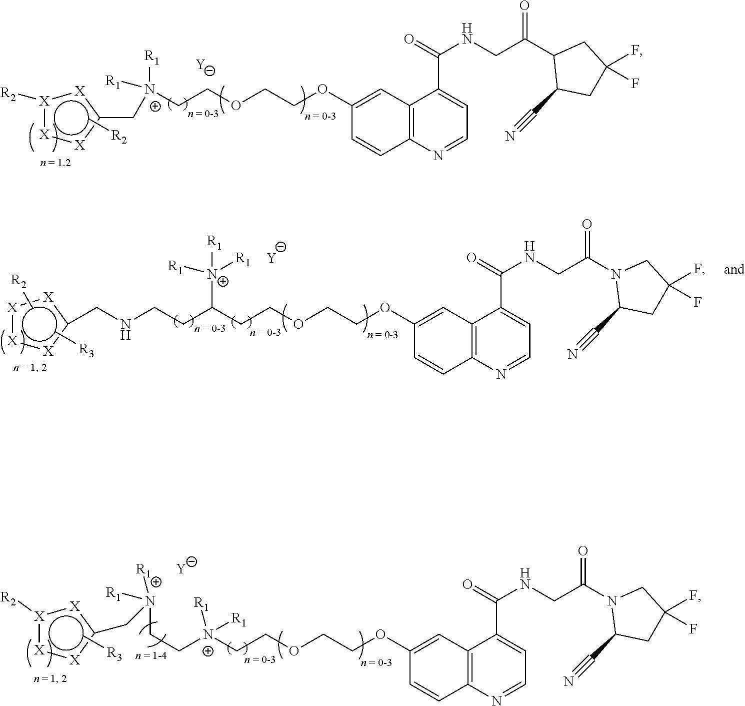

wherein Y is a counterion, preferably Cl or Br;
and wherein every n is independently selected from the range stipulated in proximity of the parenthesis. For example, for n=0-3, a selection can be made between 0, 1, 2 and 3. In an embodiment, the selected n is the lowest number suggested by the range. In an embodiment, for all n in one structure the lowest number suggested by the range is selected. In an embodiment, the selected n is the lowest number +1 suggested by the range. In an embodiment, for all n in one structure the lowest number suggested by the range +1 is selected.

In a fifth class of compounds according to an embodiment of the current invention, the compound is a compound according to Formula I or a stereoisomer, tautomer, racemic, metabolite, prodrug, salt, hydrate, or solvate thereof,
wherein Z is selected linker from the group of

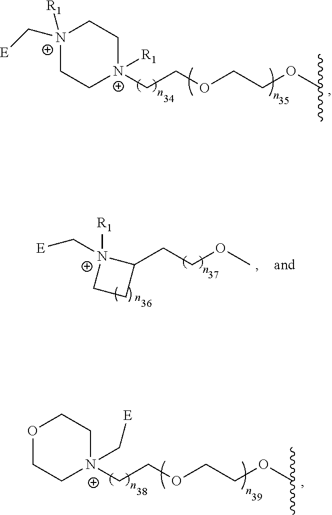

wherein
each R₁ is independently selected from the group of:
—H, —CH₃, CH₂CH₃, —CH₂CH₂CH₃, and —CH₂CH₂CH₂CH₃;
N₃₄, n₃₅, n₃₆, n₃₇, n₃₈, n₃₉ is independently 0-4;
E is

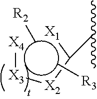

wherein each X₁, X₂, X₃, X₄ is independently selected from the group of C, O and N;
t is independently 1, 2 or 3;
each R₂ is independently selected from the group of:
¹⁸F, ¹²⁰I, ¹²²I, ¹²³I, ¹²⁴I, ¹²⁵I, ¹³¹I and ²¹¹At; and
each R₃ is independently selected from the group of:
guanidine, aminomethyl and dialkylaminomethyl.

In a further embodiment, Z is selected from the group of

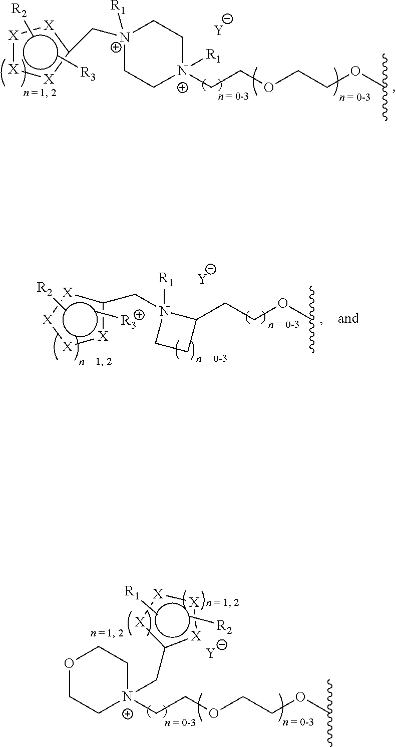

wherein
each X is independently selected from the group of C, O and N,
each R₁ is independently selected from the group of:
—H, —CH₃, CH₂CH₃, —CH₂CH₂CH₃, and —CH₂CH₂CH₂CH₃,
each R₂ is independently selected from the group of:
¹⁸F, ¹²⁰I, ¹²²I, ¹²³I, ¹²⁴I, ¹²⁵I, ¹³¹I and At, preferably ²¹¹At,
each R₃ is independently selected from the group of:
guanidine, aminomethyl and dialkylaminomethyl, preferably the dialkylaminomethyl is selected from the group of: dimethylaminomethyl, diethylaminomethyl and dipropylaminoethyl.

When selecting moieties for X, one should be aware that a 5-, 6- or 7-membered aromatic ring is formed.

Every n is independently selected from the range stipulated in proximity of the parenthesis. For example, for n=0-3, a selection can be made between 0, 1, 2 and 3. In an embodiment, the selected n is the lowest number suggested by the range. In an embodiment, for all n in one structure the lowest number suggested by the range is selected. In an embodiment, the selected n is the lowest number +1 suggested by the range. In an embodiment, for all n in one structure the lowest number suggested by the range +1 is selected.

In a fifth class of compounds according to an embodiment of the current invention, the compound is selected from the group of:

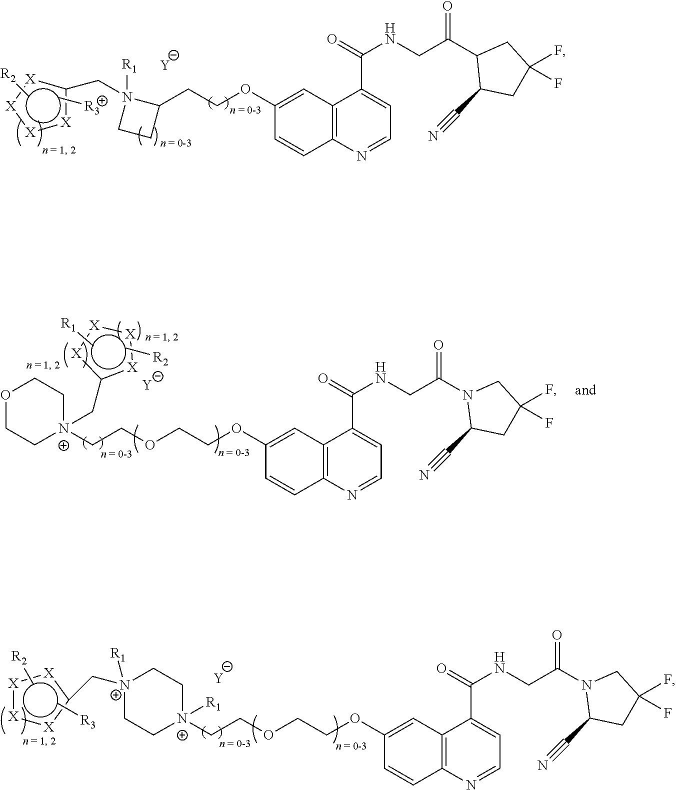

wherein Y is a counterion, preferably Cl or Br;
wherein each X is independently selected from the group of C, O and N;
wherein each R₁ is independently selected from the group of:
—H, —CH₃, CH₂CH₃, —CH₂CH₂CH₃, and —CH₂CH₂CH₂CH₃;
and wherein every n is independently selected from the range stipulated in proximity of the parenthesis. For example, for n=0-3, a selection can be made between 0, 1, 2 and 3. In an embodiment, the selected n is the lowest number suggested by the range. In an embodiment, for all n in one structure the lowest number suggested by the range is selected. In an embodiment, the selected n is the lowest number +1 suggested by the range. In an embodiment, for all n in one structure the lowest number suggested by the range +1 is selected.

In an embodiment, the compound is a compound of Formula I or a stereoisomer, tautomer, racemic, metabolite, prodrug, salt, hydrate, or solvate thereof as presented in Table 1.

TABLE 1
examples of structures according to an embodiment of the current
invention

| Compound | Chemical structure |
|---|---|
| [18F]-UAMC- 0004522 (1) | 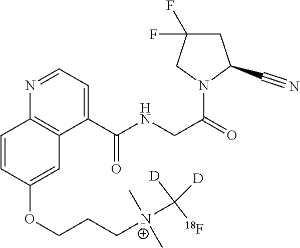 [SMILES: [2H]C([2H])([18F])[N+](C)(C)CCCOc1ccc2nccc(C(=O)NCC(=O)N3CC(F)(F)C[C@H]3C#N)c2c1] |
| UAMC-0004522 (4a) | 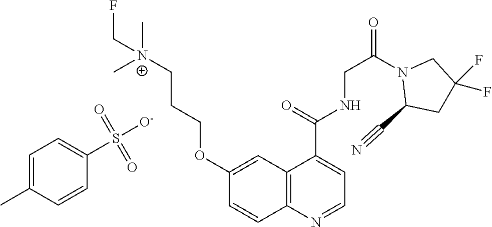 [SMILES: Cc1ccc(S(=O)(=O)[O-])cc1 ; C[N+](C)(CF)CCCOc1ccc2nccc(C(=O)NCC(=O)N3CC(F)(F)C[C@H]3C#N)c2c1] |
| UAMC-0005086 (4b) | 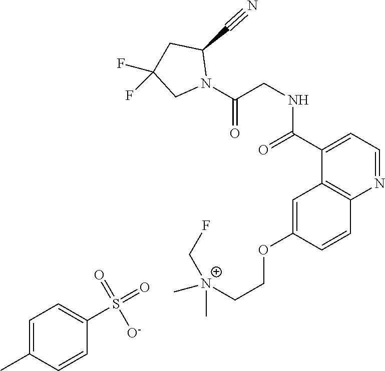 [SMILES: Cc1ccc(S(=O)(=O)[O-])cc1 ; C[N+](C)(CF)CCOc1ccc2nccc(C(=O)NCC(=O)N3CC(F)(F)C[C@H]3C#N)c2c1] |
| 4c | 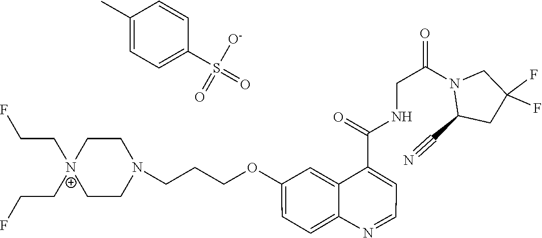 [SMILES: N#C[C@@H]1CC(F)(F)CN1C(=O)CNC(=O)c1ccnc2ccc(OCCCN3CC[N+](CCF)(CCF)CC3)cc12 ; Cc1ccc(S(=O)(=O)[O-])cc1] |
| 5 | 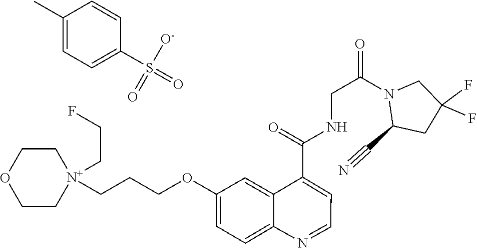 [SMILES: N#C[C@@H]1CC(F)(F)CN1C(=O)CNC(=O)c1ccnc2ccc(OCCC[N+]3(CCF)CCOCC3)cc12 ; Cc1ccc(S(=O)(=O)[O-])cc1] |
| 6 | 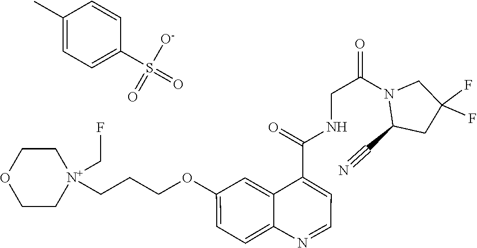 [SMILES: N#C[C@@H]1CC(F)(F)CN1C(=O)CNC(=O)c1ccnc2ccc(OCCC[N+]3(CF)CCOCC3)cc12 ; Cc1ccc(S(=O)(=O)[O-])cc1] |
| UAMC-0005258 (7) | 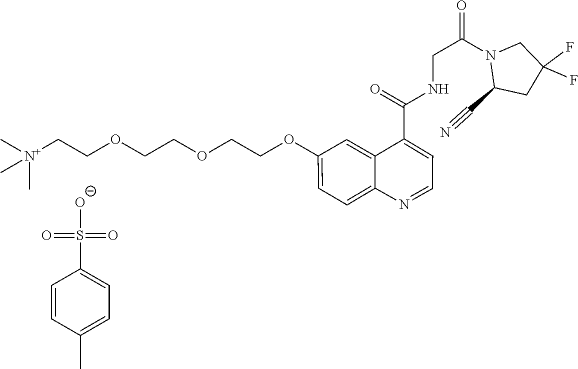 [SMILES: C[N+](C)(C)CCOCCOCCOc1ccc2nccc(C(=O)NCC(=O)N3CC(F)(F)C[C@H]3C#N)c2c1 ; Cc1ccc(S(=O)(=O)[O-])cc1] |
| UAMC-0005095 (8) | 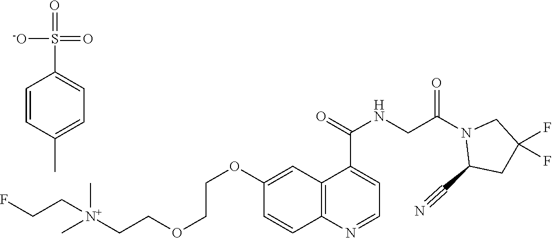 |
| UAMC-0005300 (9) | 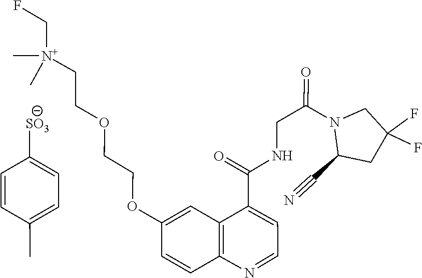 |
| UAMC-0005297 (10) | 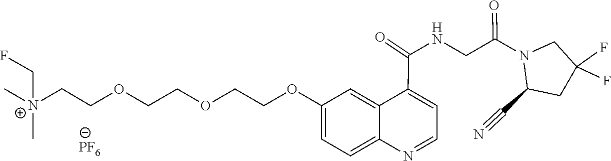 |
| UAMC-0005269 (11) | 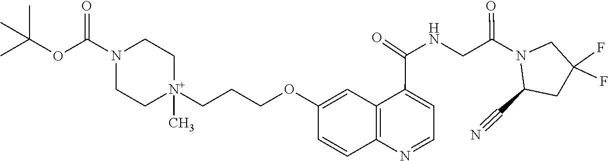 [SMILES: CC(C)(C)OC(=O)N1CC[N+](C)(CCCOc2ccc3nccc(C(=O)NCC(=O)N4CC(F)(F)C[C@H]4C#N)c3c2)CC1] |
| UAMC-0005288 (12) | 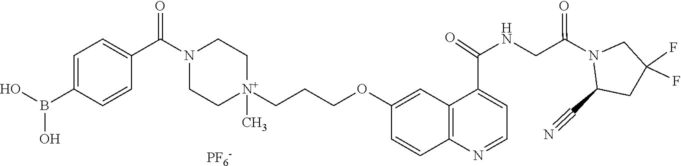 |
| UAMC-0005268 (13) | 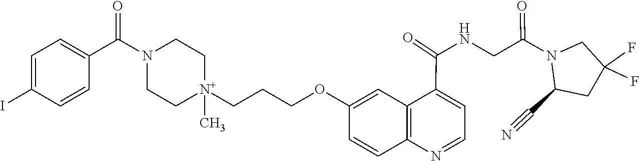 [SMILES: C[N+]1(CCCOc2ccc3nccc(C(=O)NCC(=O)N4CC(F)(F)C[C@H]4C#N)c3c2)CCN(C(=O)c2ccc(I)cc2)CC1] |
| UAMC-0005267 (14) | 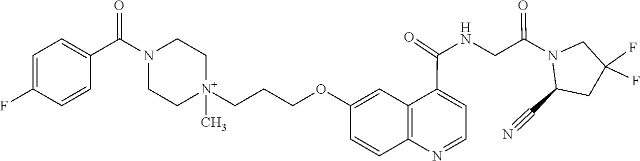 [SMILES: C[N+]1(CCCOc2ccc3nccc(C(=O)NCC(=O)N4CC(F)(F)C[C@H]4C#N)c3c2)CCN(C(=O)c2ccc(F)cc2)CC1] |

A compound as detailed herein may in one embodiment be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

The compounds depicted herein may be present as salts even if salts are not depicted and it is understood that the present disclosure embraces all salts and solvates of the compounds depicted here, as well as the non-solvate form of the compound, as is well understood by the skilled artisan. In some embodiments, the salts of the compounds provided herein are pharmaceutically acceptable salts.

In an embodiment, the compound is present as a pharmaceutically acceptable salt. Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate and the like.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein. The present disclosure also includes any or all of the stereochemical forms, including any enantiomeric or diastereomeric forms of the compounds described, such as the compounds of Table 1. The structure or name is intended to embrace all possible stereoisomers of a compound depicted. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

The invention also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or embodiments thereof described herein, where a fraction of one or more atoms are replaced by an isotope of the same element. Exemplary isotopes that can be incorporated into compounds of the invention include isotopes of hydrogen, carbon, nitrogen, oxygen, phosphorus, sulfur, halo, such as ²H, ³H, ¹³C, ¹¹C, ¹⁴C, ¹³N, ¹⁵O, ¹⁷O, ³²p, ³⁵S, ¹⁸F. Certain isotope labeled compounds (e.g. ³H and ¹⁴C) are useful in compound or substrate tissue distribution studies. Incorporation of heavier isotopes such as deuterium (²H) can cause certain therapeutic advantages resulting from greater metabolic stability, for example, increased in vivo half-life, or reduced dosage requirements and, hence may be preferred in some instances.

Isotopically-labeled compounds of the present invention can generally be prepared by standard methods and techniques known to those skilled in the art or by procedures similar to those described in the accompanying Examples substituting appropriate isotopically-labeled reagents in place of the corresponding non-labeled reagent.

### Pharmaceutical Compositions and Formulations

In a second aspect, the invention relates to a pharmaceutical composition or formulation comprising a compound as described herein and at least one pharmaceutically acceptable carrier, diluent, excipient, or adjuvant. The present invention includes pharmaceutical compositions or formulations comprising a compound as detailed herein or a salt thereof and a pharmaceutically acceptable carrier or excipient. In an embodiment, the pharmaceutically acceptable salt is an acid addition salt.

A compound according to the present invention may in one embodiment be in a purified form. In an embodiment, the composition comprises a compound as detailed herein or a salt thereof. In some embodiments, the composition comprises a compound as detailed herein or a salt thereof in substantially pure form.

In an embodiment, the compounds herein are synthetic compounds prepared for administration to an individual. In another embodiment, compositions are provided containing a compound in substantially pure form. In another embodiment, the present invention embraces pharmaceutical compositions comprising a compound detailed herein and a pharmaceutically acceptable carrier. In another embodiment, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. Pharmaceutical compositions may take a form suitable for oral, buccal, parenteral, nasal, topical or rectal administration or a form suitable for administration by inhalation. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein or a salt thereof can be used in the preparation of a composition, such as a pharmaceutical composition, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical compositions may contain preservatives, solubilizers, stabilizers, re-wetting agents, emulators, sweeteners, dyes, adjusters, and salts for the adjustment of osmotic pressure, buffers, coating agents or antioxidants. Compositions comprising the compound may also contain other substances which have valuable therapeutic properties. Pharmaceutical compositions may be prepared by known pharmaceutical methods.

Compositions as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-oils, and so on. In addition, pharmaceutical compositions may contain preservatives, solubilizers, stabilizers, re-wetting agents, emulators, sweeteners, dyes, adjusters, and salts for the adjustment of osmotic pressure, buffers, coating agents or antioxidants.

In some embodiments, the composition is for use as a human or veterinary medicament. In some embodiments, the composition is for use in a method described herein. In some embodiments, the composition is for use in the treatment of a disease or disorder described herein.

By means of non-limiting examples, such a composition may be in a form suitable for oral administration, parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person.

Some preferred, but non-limiting examples of preparations include tablets, pills, powders, lozenges, sachets, cachets, elixirs, suspensions, emulsions, solutions, syrups, aerosols, ointments, creams, lotions, soft and hard gelatin capsules, suppositories, eye drops, sterile injectable solutions and sterile packaged powders (which are usually reconstituted prior to use) for administration as a bolus and/or for continuous administration, which may be formulated with carriers, excipients, and diluents that are suitable per se for such compositions, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The compositions can optionally contain other pharmaceutically active substances (which may or may not lead to a synergistic effect with the compounds of the invention) and other substances that are commonly used in pharmaceutical compositions, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein, for example using liposomes or hydrophilic polymeric matrices based on natural gels or synthetic polymers. In order to enhance the solubility and/or the stability of the compounds of a pharmaceutical composition according to the invention, it can be advantageous to employ α-, β- or γ-cyclodextrins or their derivatives.

In addition, co-solvents such as alcohols may improve the solubility and/or the stability of the compounds. In the preparation of aqueous compositions, addition of salts of the compounds of the invention can be more suitable due to their increased water solubility.

The preparations may be prepared in a manner known per se, which usually involves mixing at least one compound according to the invention with the one or more pharmaceutically acceptable carriers, and, if desired, in combination with other pharmaceutical active compounds, when necessary, under aseptic conditions.

For an oral administration form, the compositions of the present invention can be mixed with suitable additives, such as excipients, stabilizers, or inert diluents, and brought by means of the customary methods into the suitable administration forms, such as tablets, coated tablets, hard capsules, aqueous, alcoholic, or oily solutions. Examples of suitable inert carriers are Arabic gum, magnesia, magnesium carbonate, potassium phosphate, lactose, glucose, or starch, in particular, corn starch. In this case, the preparation can be carried out both as dry and as moist granules. Suitable oily excipients or solvents are vegetal or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art. When administered by nasal aerosol or inhalation, these compositions may be prepared according to techniques well-known in the art of pharmaceutical composition and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical compositions for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the composition can also additionally contain other pharmaceutical auxiliaries such as surfactants, emulsifiers and stabilizers as well as a propellant. For subcutaneous administration, the compound according to the invention, if desired with the substances customary therefore such as solubilizers, emulsifiers or further auxiliaries are brought into solution, suspension, or emulsion. The compounds of the invention can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these compositions may be prepared by mixing the compounds according to the invention with a suitable non-irritating excipient, such as cocoa butter, synthetic glyceride esters or polyethylene glycols, which are solid at ordinary temperatures, but liquefy and/or dissolve in the rectal cavity to release the drug. In a preferred embodiment, the compounds of the present invention are useful in human or veterinary medicine, in particular for use as FAP (fibroblast activation protein) inhibitors.

In an embodiment, the pharmaceutical composition may comprise a chelator selected from the group of: EUpypa, EDTA (ethylenediamine tetraacetate), EDTMP (diethylenetriaminepenta (methylenephosphonic acid)), DTPA (diethylenetriaminepentaacetate) and its derivatives, DOTA (Dodeca-1,4,7,10-tetraamine-tetraacetate), DOTAGA (2-(I, 4,7, 10-tetraazacyclododecane-4, 7,10) pentanedioic acid) and other DOTA derivatives, TRITA (trideca-I, 4,7,10-tetraamine-tetraacetate), TETA (tetradeca-I, 4,8, II-tetraamine-tetraacetate) and its derivatives, NOTA (Nona-I, 4,7-triamine-triacetate) and its derivatives such as NOTAGA (I, 4,7-triazacyclononane, I-glutaric acid, 4,7-acetate), TRAP (triazacyclononane phosphinic acid), NOPO (I, 4,7-triazacyclononane-1,4-bis [methylene (hydroxymethyl) phosphinic acid]-7-[methylene (2-carboxyethyl) phosphinic acid]), PEPA (pentadeca-1, 4,7,10,13-pentaamine pentaacetate), NETA ({4-[2-(bis-carboxymethylamino)-ethyl]-7-carboxymethyl-[1,4,7]triazonan-1-yl}-acetic acid), 3p-C-NEPA (2-{[2-(4-{2-[Bis(carboxymethyl)amino]-5-(4-nitrophenyl)pentyl}-7-(carboxymethyl)-1,4,7-triazonan-1-yl)ethyl](carboxymethyl) amino}acetic acid), 3p-C-NETA-NCS ({4-[2-(Bis-carboxymethylamino)-5-(4-isothiocyanatophenyl) pentyl]-7-carboxymethyl-[1,4,7]triazonan-1-yl}acetic acid), HEHA (hexadeca-1, 4,7,10,13,16-hexaamine-hexaacetate) and its derivatives, HBED (hydroxybenzyl-ethylene-di amine) and its derivatives, DEDPA and its derivatives, such as H2DEDPA (I, 2-[[6-(carboxylate) pyridin-2-yl]methylamine]ethane), DFO (deferoxamine) and its derivatives, trishydroxypyridinone (THP) and its derivatives such as YM103, TEAP (tetraazycyclodecanephosphinic acid) and its derivatives, AAZTA (6-amino-6-methylperhydro-1,4-diazepine-tetraacetate) and derivatives such as DATA ((6-Pentanoic acid)-6-(amino) methyl-1,4-diazepine triacetate); SarAr (IN-(4-aminobenzyl)-3,6,10,13,16,19-hexaazabicyclo [6.6.6]-eicosane-1,8-diamine) and salts thereof, (Nhh SAR (1,8-diamino-B, 6,10,13,16,19-hexaazabicyclo [6.6.6] icosane) and salts and derivatives thereof, aminothiols and their derivatives.

### Methods of Treatment and Use of Compounds or Compositions

Also disclosed herein are methods of treatment and/or diagnostic methods. More in particular, disclosed herein are compounds or pharmaceutical composition as disclosed herein for use for the treatment and/or the diagnosis of a disease.

In an embodiment, a method for treating a disease in an individual is disclosed herein, wherein said disease is a FAP-related disorder.

Said FAP-related disorder is preferably selected from the list comprising proliferative diseases selected from the group of breast cancer, colorectal cancer, ovarian cancer, prostate cancer, pancreatic cancer, kidney cancer, lung cancer, melanoma, fibrosarcoma, bone and connective tissue sarcomas, renal cell carcinoma, giant cell carcinoma, squamous cell carcinoma, and adenocarcinoma; diseases characterized by tissue remodeling and/or chronic inflammation such as fibrotic diseases, wound healing disorders, keloid formation disorders, osteoarthritis, rheumatoid arthritis, cartilage degradation disorders, atherosclerotic disease and Crohn's disease; disorders involving endocrinological dysfunction, such as disorders of glucose metabolism; and blood clotting disorders.

The term “FAP-related disorder” as used herein, means any disease or other deleterious condition in which FAP is known to play a role. The term “FAP-related disorder” also means those diseases or conditions that are alleviated by treatment with a FAP inhibitor. A non-limiting list of examples of FAP-related disorders can include proliferative diseases selected from the group of breast cancer, colorectal cancer, ovarian cancer, prostate cancer, pancreatic cancer, kidney cancer, lung cancer, melanoma, fibrosarcoma, bone and connective tissue sarcomas, renal cell carcinoma, giant cell carcinoma, squamous cell carcinoma, leukemia, skin cancer, soft tissue cancer, liver cancer, gastrointestinal carcinoma, and adenocarcinoma. In addition, the list of FAP-related disorders that are envisaged here, includes diseases characterized by tissue remodeling and/or chronic inflammation. These include but are not limited to fibrotic disease, wound healing, keloid formation, osteoarthritis, rheumatoid arthritis and related disorders involving cartilage degradation, atherosclerotic disease, Type II diabetes and Crohn's disease. Furthermore, FAP related disorders involving endocrinological dysfunction (including but not limited to disorders of glucose metabolism) and diseases involving blood clotting disorders are part of this list. The invention also provides methods for the prevention and/or treatment of a FAP-related disorder; said method comprising administering to a subject in need thereof a compound according to this invention, or a composition comprising said compound.

In an embodiment, a compound or salt thereof described herein or a composition described herein may be used in a method of treating a disease or disorder mediated by FGF21. In some embodiments, a compound or salt thereof described herein or a composition described herein may be used in a method of treating a FGF21-associated disorder, such as obesity, type I-and type II diabetes, pancreatitis, dyslipidemia, hyperlipidemia conditions, non-alcoholic fatty liver disease (NAFLD), non-alcoholic steatohepatitis (NASH), insulin resistance, hyperinsulinemia, glucose intolerance, hyperglycemia, metabolic syndrome, acute myocardial infarction, hypertension, cardiovascular diseases, atherosclerosis, peripheral arterial disease, apoplexy, heart failure, coronary artery heart disease, renal disease, diabetic complications, neuropathy, gastroparesis, disorder associated with a serious inactivation mutation in insulin receptor, and other metabolic disorders. In some embodiments, the FGF21-associated disorder is diabetes, obesity, dyslipidemia, metabolic syndrome, non-alcoholic fatty liver disease, non-alcoholic steatohepatitis or cardiovascular diseases. Provided herein is a method of increasing the level of FGF21 expression in an individual comprising administering to the individual a compound described herein, or a pharmaceutically acceptable salt thereof, or a pharmaceutical composition described herein. FGF21 stimulates glucose uptake in adipocytes and is believed to protective against obesity and insulin insensitivity. By way of example and not wishing to be bound by theory, FAP is believed to be the enzyme responsible for cleavage and inactivation of FGF21; therefore, inhibiting FAP may increase levels of FGF21 expression. Accordingly, provided herein are methods of treating diabetes mellitus, insulin insensitivity, and/or obesity in an individual in need thereof comprising administering to the individual a compound described herein, or a pharmaceutically acceptable salt thereof, or a pharmaceutical composition described herein, wherein FGF21 expression is increased. In some embodiments, the diabetes mellitus is type II diabetes.

Also provided herein is a method of enhancing an immune response in an individual comprising administering to the individual a compound described herein, or a pharmaceutically acceptable salt thereof, or a pharmaceutical composition described herein. In some embodiments, the individual has cancer. In some embodiments, the enhanced immune response is directed to a tumor or cancerous cell. By way of example and not wishing to be bound by theory, FAP is believed to suppress immune responses, especially in the context of cancer, therefore inhibiting FAP may enhance the immune response of an individual. Accordingly, provided herein are methods of treating cancer in an individual in need thereof comprising administering to the individual a compound described herein, or a pharmaceutically acceptable salt thereof, or a pharmaceutical composition described herein, wherein an immune response of the individual is increased.

In an embodiment, a compound or salt thereof described herein or a composition described herein may be used in a method of treating a disease or disorder characterized by proliferation, tissue remodeling, fibrosis, chronic inflammation, excess alcohol consumption, or abnormal metabolism. In some embodiments, a compound or salt thereof described herein or a composition described herein may be used in a method of treating cancer, such as breast cancer, colorectal cancer, ovarian cancer, prostate cancer, pancreatic cancer, kidney cancer, lung cancer, melanoma, fibrosarcoma, bone sarcoma, connective tissue sarcoma, renal cell carcinoma, giant cell carcinoma, squamous cell carcinoma, leukemia, skin cancer, soft tissue cancer, liver cancer, gastrointestinal carcinoma, or adenocarcinoma. In some embodiments, the compound, salt, or composition may be used in a method of treating metastatic kidney cancer, chronic lymphocytic leukemia, pancreatic adenocarcinoma, or non-small cell lung cancer

Compounds and compositions detailed herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays. Provided herein is a method of treating a disease or disorder in an individual in need thereof comprising administering a compound describes herein or any embodiment, or aspect thereof, or a pharmaceutically acceptable salt thereof. In some embodiments, the compound, pharmaceutically acceptable salt thereof, or composition is administered to the individual according to a dosage and/or method of administration described herein.

In an embodiment, the administration of the compound, salt, or composition reduces tumor growth, tumor proliferation, or tumorigenicity in the individual. In some embodiments, the compound, salt, or composition may be used in a method of reducing tumor growth, tumor proliferation, or tumorigenicity in an individual in need thereof. In some embodiments, tumor growth is slowed or stopped. In some embodiments, tumor growth is reduced at least about 10%, 20%, 30%, 40%, 50%, 60%, 70%, 80%, 90% or more. In some embodiments, the tumor is reduced in size. In some embodiments, tumor metastasis is prevented or slowed.

In some embodiments, a compound or salt thereof described herein or a composition described herein may be used in in a method of treating fibrotic disease, thrombosis, wound healing, keloid formation, osteoarthritis, rheumatoid arthritis and related disorders involving cartilage degradation, atherosclerotic disease, Crohn's disease, hepatic cirrhosis, idiopathic pulmonary fibrosis, myocardial hypertrophy, diastolic dysfunction, obesity, glucose intolerance, insulin insensitivity, or diabetes mellitus. In some embodiments, hepatic cirrhosis is viral hepatitis-induced, alcohol-induced, or biliary cirrhosis. In some embodiments, diabetes mellitus is type II diabetes. In some embodiments, the disease or disorder is fibrotic liver degeneration.

In some embodiments, provided herein is a method of inhibiting FAP. The compounds or salts thereof described herein and compositions described herein are believed to be effective for inhibiting FAP. In some embodiments, the method of inhibiting FAP comprises inhibiting FAP in a cell by administering or delivering to the cell a compound described herein, or a pharmaceutically acceptable salt thereof, or a pharmaceutical composition described herein. In some embodiments, the cell is a fibroblast, such as a myofibroblast, a keloid fibroblast, a cancer associated fibroblast (CAF), or a reactive stromal fibroblast, among others ceils with FAP expression. In some embodiments, the method of inhibiting FAP comprises inhibiting FAP in a tumor or in plasma by administering or delivering to the tumor or plasma a compound described herein, or a pharmaceutically acceptable salt thereof, or a pharmaceutical composition described herein. In some embodiments, the inhibition of FAP comprises inhibiting an endopeptidase and/or exopeptidase activity of FAP. In some embodiments, FAP is inhibited by at least about 10%, 20%, 30%, 40%, 50%, 60%, 70%, 80%, 90%, 95%, 98% or more. Inhibition of FAP can be determined by methods known in the art.

In some embodiments, (a) a compound as described herein, or a pharmaceutically acceptable salt thereof, or a pharmaceutical composition described herein and (b) an additional agent are sequentially administered, concurrently administered or simultaneously administered. In certain embodiments, (a) a compound as described herein, or a pharmaceutically acceptable salt thereof, or a pharmaceutical composition described herein and (b) an additional agent are administered with a time separation of about 15 minutes or less, such as about any of 10, 5, or 1 minutes or less. In certain embodiments, (a) a compound described herein, or a pharmaceutically acceptable salt thereof, or a pharmaceutical composition described herein and (b) an additional agent are administered with a time separation of about 15 minutes or more, such as about any of 20, 30, 40, 50, 60, or more minutes. Either (a) a compound described herein, or a pharmaceutically acceptable salt thereof, or a pharmaceutical composition described herein and (b) an additional agent may be administered first. In certain embodiments, (a) a compound described herein, or a pharmaceutically acceptable salt thereof, or a pharmaceutical composition described herein and (b) an additional agent are administered simultaneously.

In some embodiments, the agent targets an immune checkpoint protein. In some embodiments, the agent is an antibody that targets an immune checkpoint protein. In some embodiments, the additional agent targets PD-1, PD-L1, PD-L2, CTLA4, TIMS, LAGS, CCR4, OX40, OX40L, IDO, and A2AR. In some embodiments, the agent is an anti-PD-I antibody, an anti-PD-LI antibody, or an anti-CTL.4-4 antibody.

The compounds as disclosed herein show high selectivity and can be used to demonstrate the presence of cancer cells or cancer associated fibroblasts. Hence, use of the compounds or compositions as described herein in diagnostics is also covered by the current invention.

The compounds of the current invention, once administered to the patient, can localize to specific organs or cells allowing visualizing the extent of a disease-process in the body, based on the cellular function and physiology, rather than relying on physical changes in the tissue anatomy. The involvement of FAP in many physiological or physio-pathological aspects offers the possibility of targeting these cells, to obtain an early diagnosis by a non-invasive approach.

Also disclosed herein are methods of imaging tissues, organs and/or cell populations by the compounds and/or compositions described herein. The invention also relates to compounds or pharmaceutical compositions as described herein for use in tissue and/or organ imaging. The invention also relates to compounds or pharmaceutical compositions as described herein for use as a companion diagnostic. In an embodiment, methods of assessing whole-body target expression are disclosed herein. These methods can contribute to the evaluation and development of FAP-targeting agents: small molecules, mAbs, ADCs, BiTEs, and radionuclide therapy.

In an embodiment, methods of measuring tumor growth, tumor proliferation, and tumorigenicity are disclosed herein, for example by repeated imaging of the individual. In an embodiment, said tumor growth, tumor proliferation, or tumorigenicity is compared to the tumor growth, tumor proliferation, or tumorigenicity in the individual prior to the administration of the compound, salt, or composition. In some embodiments, the tumor growth, tumor proliferation, or tumorigenicity is compared to the tumor growth, tumor proliferation, or tumorigenicity in a similar individual or group of individuals.

In an embodiment, the compounds as described herein comprise a radionuclide, preferably ¹⁸F, ¹²⁰I, ¹²²I, ¹²³I, ¹²⁴I, ¹²⁵I, ¹³¹I and ²¹¹At. The compound comprising a radionuclide shows a high affinity for tissues characterized by significant FAP-expression, for example tumor tissue and limited accumulation in normal tissues. Furthermore, the compounds as described herein improve the contrast or signal-to-noise ratio in the imaging PET diagnostics of tumors. The radiation exposure to the targeted cells and neighboring organs is also reduced, which represents a considerable advantage for theragnostic treatment.

In an embodiment, the compound described above is used for the production of a radiopharmaceutical for imaging diagnostics by means of positron emission tomography (PET). In an embodiment, the compound described above is used for the production of a radiopharmaceutical for imaging diagnostics by means of single-photon emission computed tomography (SPECT).

In some embodiments, the individual is an animal, preferably a mammal. In some embodiments, the individual is a primate, bovine, ovine, porcine, equine, canine, feline, or rodent. In some embodiments, the individual is a human. In some embodiments, the individual has any of tire diseases or disorders disclosed herein. In some embodiments, the individual is a risk for developing any of the diseases or disorders disclosed herein. In some embodiments, the individual is human. In some embodiments, the human is at least about or is about any of 21, 25, 30, 35, 40, 45, 50, 55, 60, 65, 70, 75, 80, or 85 years old. In some embodiments, the human is a child. In some embodiments, the human is less than about or about an age of 21, 18, 15, 12, 10, 8, 6, 5, 4, 3, 2, or 1 years old.

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The pharmaceutical compositions of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300 or 400 mg per unit dosage.

The compounds can be administered by a variety of routes including the oral, rectal, ocular, transdermal, subcutaneous, intravenous, intramuscular or intranasal routes, depending mainly on the specific preparation used and the condition to be treated or prevented, and with oral and intravenous administration usually being preferred. At least one compound of the invention will generally be administered in an “effective amount”, by which is meant any amount of a compound of the Formula I, upon suitable administration, is sufficient to achieve the desired therapeutic or prophylactic effect in the individual to which it is administered. Usually, depending on the condition to be prevented or treated and the route of administration, such an effective amount will usually be between 0.01 to 1000 mg per kilogram body weight day of the patient per day, more often between 0.1 and 500 mg, such as between 1 and 250 mg, for example about 5, 10, 20, 50, 100, 150, 200 or 250 mg, per kilogram body weight day of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated.

The effective amount of the compound may in one embodiment be a dose of between about 0.01 and about 100 mg/kg. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

A compound or composition of the invention may be administered to an individual in accordance with an effective dosing regimen for a desired period of time or duration, such as at least about one month, at least about 2 months, at least about 3 months, at least about 6 months, or at least about 12 months or longer, which in some embodiments may be for the duration of the individual's life. In one embodiment, the compound is administered on a daily or intermittent schedule. The compound can be administered to an individual continuously (for example, at least once daily) over a period of time. The dosing frequency can also be less than once daily, e.g., about once-weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

In an embodiment, said pharmaceutical composition can be administered separately at different times during the course of therapy or concurrently in divided or single combination forms.

### Articles of Manufacture and Kits

The present disclosure further provides articles of manufacture comprising a compound described herein or a salt thereof, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the invention, which comprises one or more compounds described herein or a composition comprising a compound described herein. The kits may employ any of the compounds disclosed herein. In one embodiment, the kit employs a compound described herein or a salt thereof. The kits may be used for any one or more of the uses described herein, and, accordingly, may contain instructions for the treatment any disease or described herein, for example for the treatment of cancer. Kits generally comprise suitable packaging. The kits may comprise one or more containers comprising any compound described herein. Each component (if there is more than one component) can be packaged in separate containers or some components can be combined in one container where cross-reactivity and shelf-life permit. The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 wrecks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

### Synthesis

Compounds of formula I can be prepared as indicated in general Scheme 1.

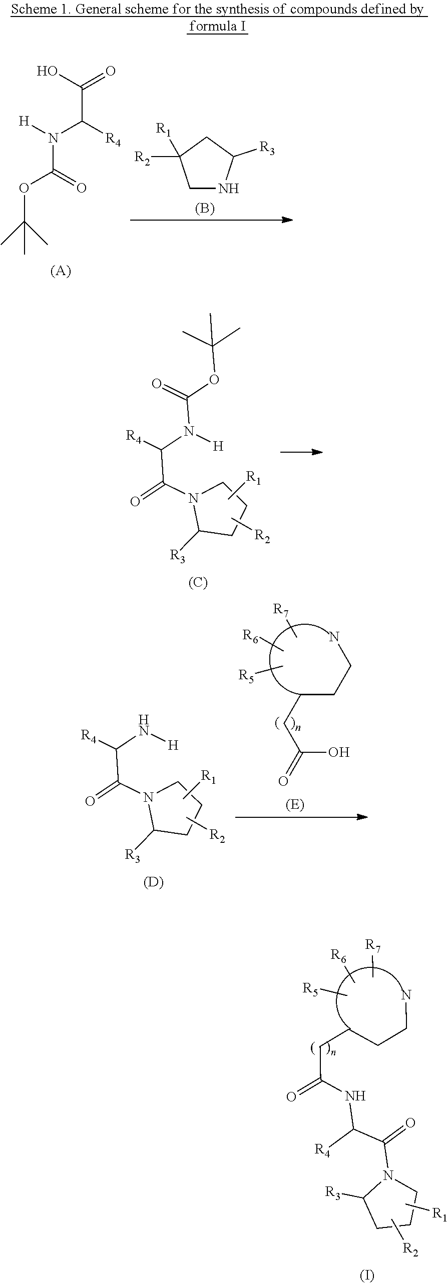

This can be carried out as mentioned below.

The reaction of Boc-aminocarboxylate (A) with compound (B) can be done using standard techniques for peptide coupling, known to a person skilled in the art, to afford compound (C). This is either pyrrolidine or a pyrrolidine derivative. In cases where the pyrrolidine is a pyrrolidinecarboxamide an additional dehydration step (e.g., using trifluoroacetic anhydride and pyridine, vide infra) has to follow the coupling step. The chosen protecting group of (C), including the Boc- and Z-group can be deprotected using a suitable acid or other deprotecting agent, known to a person skilled in the art, to generate compound (D).

The reaction of compound (D) with compound (E) can be done using standard peptide coupling procedures, or using a corresponding acyl halide or active ester of (E) which is made in situ or in a separate reaction using procedures known to a person skilled in the art.

Alternatively, compounds of formula (I) can also be prepared by converting one or more groups R₁, R₂, R₃, R₄, R₅, R₆ or R₇ of another compound of formula I obtained as mentioned above, into a desired substituent. The selected conversion method will depend on the kind of substituents desired.

The invention is further described by the following non-limiting examples which further illustrate the invention, and are not intended to, nor should they be interpreted to, limit the scope of the invention.

### EXAMPLES

Synthetic approach, procedures and compound characterization for compounds presented in Table 1.

### 1) Synthetic Approach, Procedures and Compound Characterization for Non-Radioactive Products (Compounds 1-14)

The overall synthetic approach to the described quaternary ammonium-containing molecules, is shown in Scheme 1. 6-Hydroxyquinoline-4-carboxylic acid is used as a common starting material and first transformed into the corresponding chloroethyl or chloropropyl or BoC-amine PEG derivatives ether via O-alkylation and subsequent saponification. After amination with the required secondary amine (either dimethylamine, morpholine or N-Boc-piperazine), or removal of the Boc-group and methylation (more specifically demethylation) of the primary amine, the glycine-(2-cyano-4,4-difluoropyrrolidine) moiety is installed through COMU or HATU coupling. The quaternary ammonium group is installed through alkylation, which in the case of the N-Boc-piperazinyl derivative is either preceded by trifluoroacetic acid mediated deprotection of the Boc-group, or the Boc-group deprotection is performed after quaternary ammonium formation and it is followed by amide coupling with the corresponding benzoic acid. The alkylation either involves fluoromethyl tosylate or fluoroethyl tosylate or iodomethane.

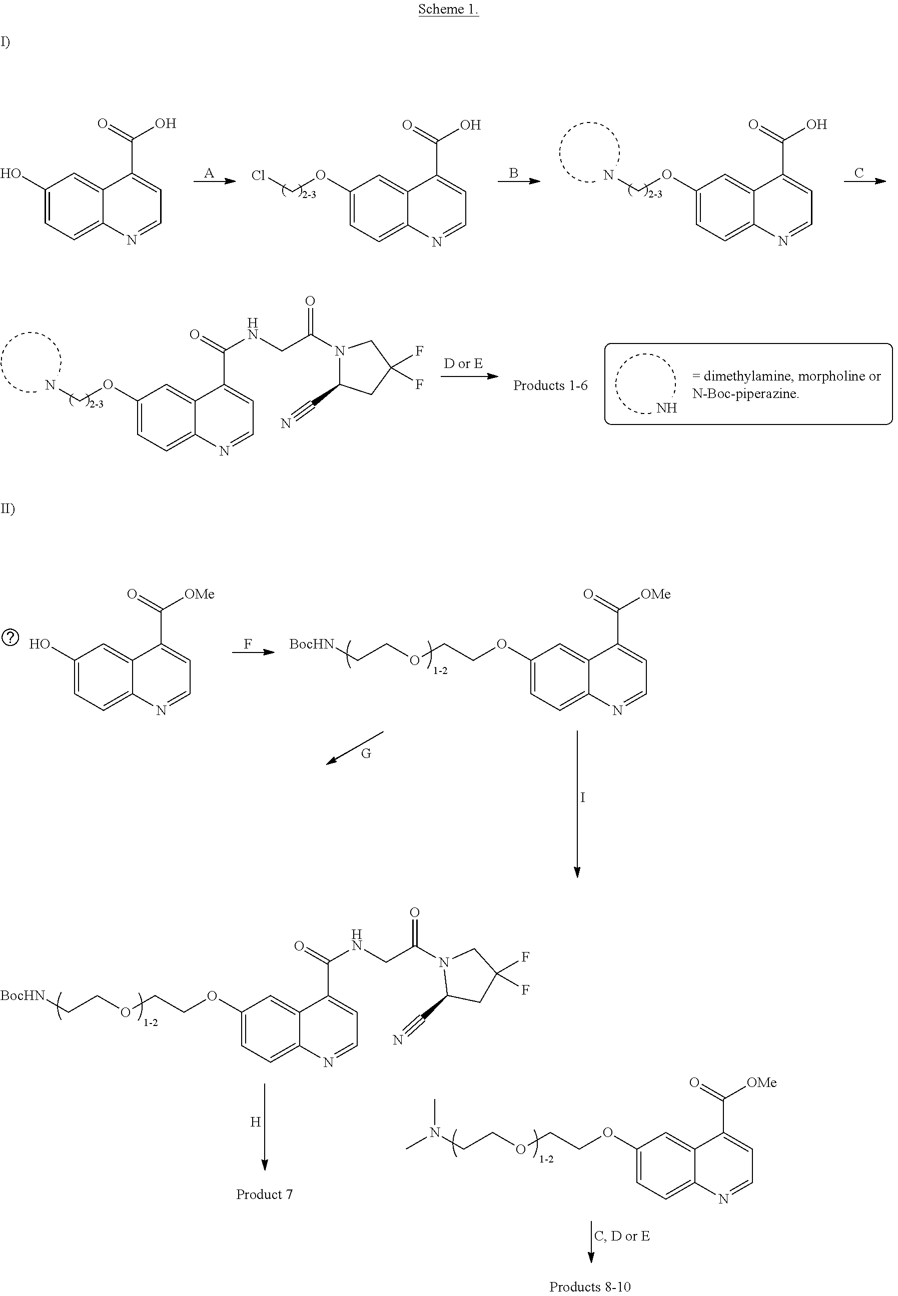

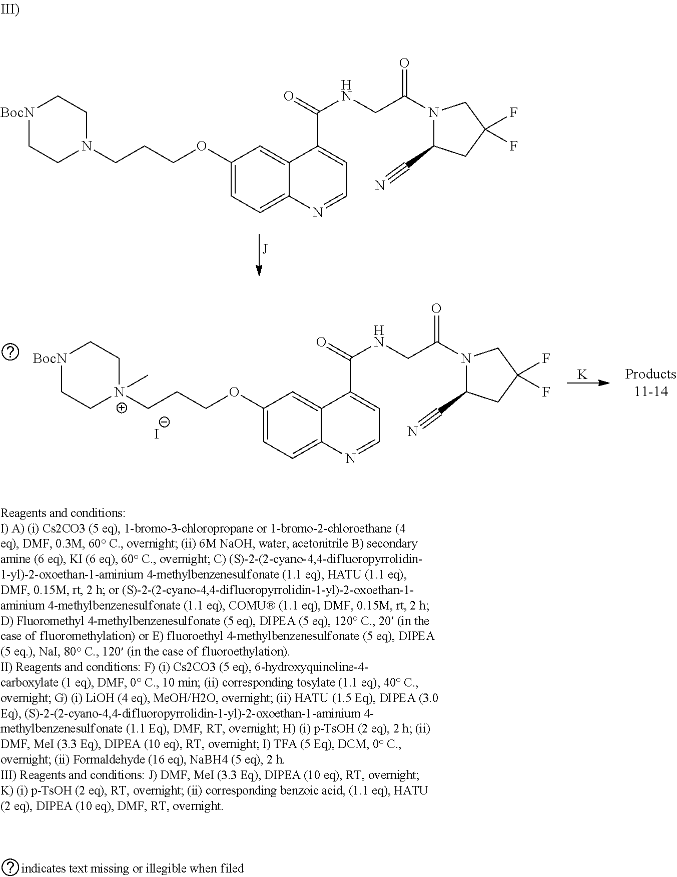

General Procedure a (Nucleophilic Substitution with Linker)

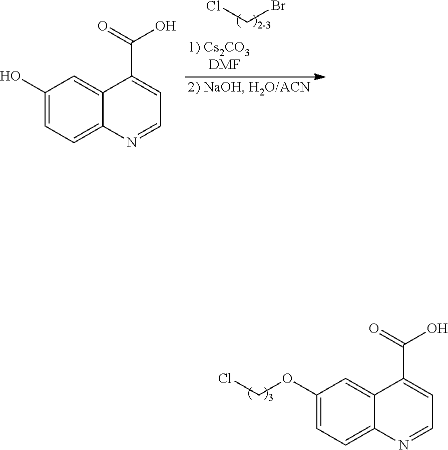

6-Hydroxyquinoline-4-carboxylic acid (1 eq) was stirred under argon atmosphere at 50° C. with Cs₂CO₃ (5 eq) in dry DMF (reaction molarity≈0.3 M) for 20′. The bromochloroalkane (4 eq) was added and the mixture was stirred at 60° C. overnight. The mixture was filtered under vacuum and the solids were washed with 80 mL of EtOAc. The filtrate was washed with 20 mL of water and 2×25 mL of brine. The aqueous phases were discarded and the organic fraction was evaporated under reduced pressure. The obtained residue was diluted with 12 mL of water and 25 mL of acetonitrile, then 5 mL of NaOH 6M were added and the mixture was stirred until complete hydrolysis of the ester (around 1 h30′). The mixture spontaneously separated into a top organic layer and a bottom aqueous layer. The aqueous layer was set aside and the organic layer was evaporated via rotary evaporator. The residue was redissolved in 15 mL of DCM and extracted with 15 mL of water. The combined water phases were washed with 2×25 mL of diethyl ether and subsequently titrated with HCl 6M to pH 1, forming a milky beige precipitate that was collected with a glass filter and dried under reduced pressure.

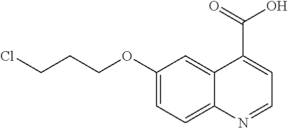 [SMILES: O=C(O)c1ccnc2ccc(OCCCCl)cc12]

6-(3-chloropropoxy)quinoline-4-carboxylic acid (1a) (2.289 mg, 8.62 mmol, 65% yield) was prepared according to general procedure A with 1-Bromo-3-chloropropane (5.23 ml, 52.9 mmol). MS (ESI): m/z=266.0 [M+H]⁺ (³⁵Cl); 268.0 [M+H]⁺ (³⁷Cl). ¹H NMR (400 MHz, DMSO) δ 8.87 (d, J=4.4 Hz, 1H), 8.19 (d, J=2.8 Hz, 1H), 8.03 (d, J=9.2 Hz, 1H), 7.93 (d, J=4.4 Hz, 1H), 7.52 (dd, J=9.2, 2.7 Hz, 1H), 4.24 (t, J=6.0 Hz, 2H), 3.86 (t, J=6.5 Hz, 2H), 2.27 (p, J=6.2 Hz, 2H).

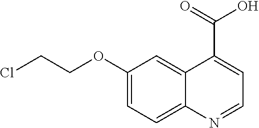 [SMILES: O=C(O)c1ccnc2ccc(OCCCl)cc12]

6-(2-chloroethoxy)quinoline-4-carboxylic acid (1b) (282 mg, 1.121 mmol, 42.4% yield) was prepared according to general procedure A with 1-bromo-2-chloroethane (0.872 mL, 10.57 mmol). MS (ESI): m/z=252.0 [M+H]⁺ (³⁵Cl); 254.1 [M+H]⁺ (³⁷Cl). ¹H NMR (400 MHz, DMSO) δ 13.83 (s, 1H), 8.89 (d, J=4.4 Hz, 1H), 8.20 (d, J=2.8 Hz, 1H), 8.05 (d, J=9.2 Hz, 1H), 7.94 (d, J=4.4 Hz, 1H), 7.55 (dd, J=9.2, 2.8 Hz, 1H), 4.40 (t, J=5.1 Hz, 2H), 4.05 (t, J=5.2 Hz, 2H).

General Procedure B (Nucleophilic Substitution with Amine)

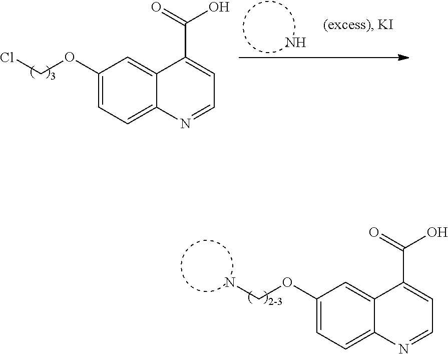

A suspension of 6-(chloroalkoxy)quinoline-4-carboxylic acid (1 eq), potassium iodide (6 eq), and the corresponding secondary amine (6 eq) in DMF (reaction molarity≈0.3 M) was stirred at 60° C. overnight. The crude mixture was filtered under vacuum and the solids were washed with ACN. 2 Eq of aqueous KOH 6M were added to the filtrate before evaporating it under reduced pressure. The obtained mass was triturated with 3×50 mL diethyl ether and used without further purification in the following reactions.

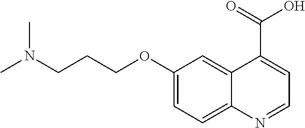 [SMILES: CN(C)CCCOc1ccc2nccc(C(=O)O)c2c1]

6-(3-(dimethylamino)propoxy)quinoline-4-carboxylic acid (2a) was prepared according to general procedure B with 6-(3-chloropropoxy)quinoline-4-carboxylic acid (1000 mg, 3.01 mmol) and dimethylamine 2M solution in THF (8.62 mL, 17.24 mmol). MS (ESI): m/z=275.1 [M+H]⁺.

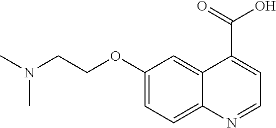 [SMILES: CN(C)CCOc1ccc2nccc(C(=O)O)c2c1]

6-(2-(dimethylamino)ethoxy)quinoline-4-carboxylic acid (2b) was prepared according to general procedure B with 6-(2-chloroethoxy)quinoline-4-carboxylic acid (90 mg, 0.358 mmol) and dimethylamine 2M solution in THF (1028 μl, 2.056 mmol). MS (ESI): m/z=261.2 [M+H]⁺.

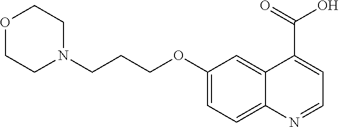 [SMILES: O=C(O)c1ccnc2ccc(OCCCN3CCOCC3)cc12]

6-(3-morpholinopropoxy)quinoline-4-carboxylic acid (2c) (119 mg, 0.376 mmol, 20% yield) was prepared according to general procedure B with 6-(3-chloropropoxy)quinoline-4-carboxylic acid (500 mg, 1,882 mmol) and morpholine (0.974 mL, 11.29 mmol). MS (ESI): m/z=317.1 [M+H]⁺.

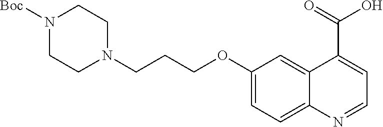 [SMILES: CC(C)(C)OC(=O)N1CCN(CCCOc2ccc3nccc(C(=O)O)c3c2)CC1]

6-(2-morpholinoethoxy)quinoline4-carboxylic acid (2d) was prepared according to general procedure B with 6-(2-chloroethoxy)quinoline-4-carboxylic acid (282 mg, 1.121 mmol) and Morpholine (198 μl, 2.296 mmol). MS (ESI): m/z=401.2 [M+H]⁺.

### General Procedure C (Amide Coupling)

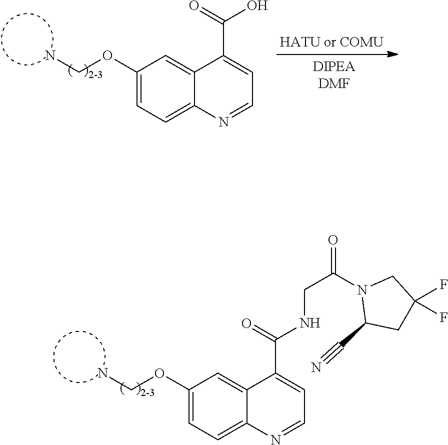

A 10 mL round bottom flask was charged with the corresponding carboxylic acid (1 eq), HATU (1.1 eq) or COMU® (3.0 eq), dry DMF (reaction molarity≈0.15 M) and dry DIPEA (5 eq). After 5 minutes stirring at room temperature, (S)-2-(2-cyano-4,4-difluoropyrrolidin-1-yl)-2-oxoethan-1-aminium 4-methylbenzenesulfonate (1.1 eq) was added. After the consumption of the starting materials (UPLC monitoring) all volatiles removed via rotary evaporator. The crude was diluted with 5 mL of DCM washed with saturated aqueous sodium bicarbonate (3×1 mL), dried over Na₂SO₄, filtered and the solvent was eliminated under vacuo to give a residue that was purified by reverse-phase column chromatography eluting with a gradient of acetonitrile (ACN) in water.

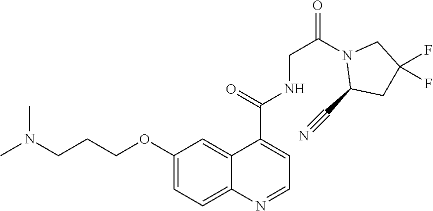 [SMILES: CN(C)CCCOc1ccc2nccc(C(=O)NCC(=O)N3CC(F)(F)C[C@H]3C#N)c2c1]

(S)—N-(2-(2-cyano-4,4-difluoropyrrolidin-1-yl)-2-oxoethyl)-6-(3-(dimethylamino)propoxy)quinoline-4-carboxamide (3a) (42.2 mg, 0.095 mmol, 4% yield) was prepared according to general procedure C with 6-(3-(dimethylamino)propoxy)quinoline-4-carboxylic acid (642 mg, 2.340 mmol) and HATU. The product was further purified via RP-HPLC with a gradient of acetonitrile in water+0.1% formic acid (5-50% in 20′). MS (ESI): m/z=223.7 [M+2H]⁺⁺; 446.3 [M+H]⁺; 468.2 [M+Na]⁺. ¹H NMR (400 MHz, DMSO) δ 9.13 (t, J=6.0 Hz, 1H), 8.83 (d, J=4.3 Hz, 1H), 8.01 (d, J=9.2 Hz, 1H), 7.89 (d, J=2.8 Hz, 1H), 7.54 (d, J=4.3 Hz, 1H), 7.47 (dd, J=9.2, 2.8 Hz, 1H), 5.15 (dd, J=9.3, 3.0 Hz, 1H), 4.34 (m, 1H), 4.24 (d, J=6.3 Hz, 2H), 4.21 (d, J=6.0 Hz, 2H), 4.15 (m, 1H), 3.17 (m, 2H), 2.92 (m, 2H), 2.75 (s, 6H), 2.16 (p, J=7.8 Hz, 2H).

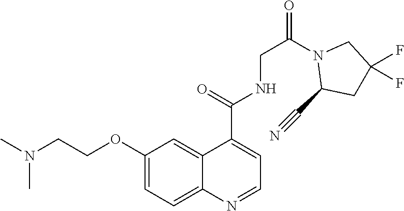 [SMILES: CN(C)CCOc1ccc2nccc(C(=O)NCC(=O)N3CC(F)(F)C[C@H]3C#N)c2c1]

(S)—N-(2-(2-cyano-4,4-difluoropyrrolidin-1-yl)-2-oxoethyl)-6-(2-(dimethylamino)ethoxy)quinoline-4-carboxamide (3b) (42.2 mg, 0.098 mmol, 27.4% yield) was prepared according to general procedure C with 6-(2-(dimethylamino)ethoxy)quinoline-4-carboxylic acid (93 mg, 0.357 mmol) and HATU. MS (ESI): m/z=432.3 [M+H]⁺; 216.7[M+2H]²⁺.

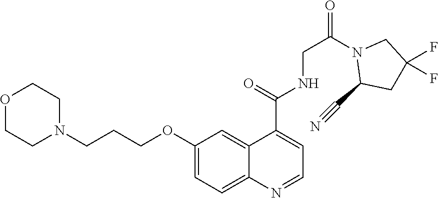 [SMILES: N#C[C@@H]1CC(F)(F)CN1C(=O)CNC(=O)c1ccnc2ccc(OCCCN3CCOCC3)cc12]

(S)—N-(2-(2-cyano-4,4-difluoropyrrolidin-1-yl)-2-oxoethyl)-6-(3-morpholinopropoxy)quinoline-4-carboxamide (3c) (75 mg, 0.154 mmol, 25% yield) was prepared according to general procedure C with 6-(3-morpholinopropoxy)quinoline-4-carboxylic acid (400 mg, 0.607 mmol) and COMU®. MS (ESI): m/z=488.1 [M+H]⁺; 244.6[M+2H]²⁺; ¹H NMR (400 MHz, MeOD) δ 8.73 (d, J=4.5 Hz, 1H), 7.95 (d, J=9.2 Hz, 1H), 7.90 (d, J=2.7 Hz, 1H), 7.55 (d, J=4.4 Hz, 1H), 7.44 (dd, J=9.2, 2.7 Hz, 1H), 5.13 (dd, J=9.4, 2.9 Hz, 1H), 4.19 (m, 6H), 3.77 (t, J=4.7 Hz, 4H), 2.88 (m, 8H), 2.15 (dp, J=12.2, 5.4 Hz, 2H).

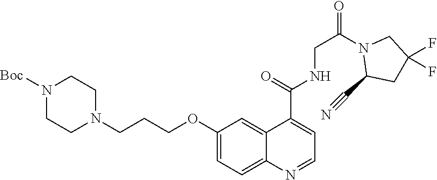 [SMILES: CC(C)(C)OC(=O)N1CCN(CCCOc2ccc3nccc(C(=O)NCC(=O)N4CC(F)(F)C[C@H]4C#N)c3c2)CC1]

Tert-butyl (S)-4-(3-((4-((2-(2-cyano-4,4-difluoropyrrolidin-1-yl)-2-oxoethyl)carbamoyl)quinolin-6-yl)oxy)propyl)piperazine-1-carboxylate (3d) (42.2 mg, 0.098 mmol, 27% yield) was prepared according to general procedure C with 6-(2-(dimethylamino)ethoxy)quinoline-4-carboxylic acid (93 mg, 0.357 mmol) and HATU. MS (ESI): m/z=587.3 [M+H]⁺. ¹H NMR (400 MHz, DMSO) δ 9.11 (t, J=6.1 Hz, 1H), 8.80 (d, J=4.4 Hz, 1H), 7.97 (d, J=9.2 Hz, 1H), 7.87 (d, J=2.8 Hz, 1H), 7.50 (d, J=4.4 Hz, 1H), 7.45 (dd, J=9.2, 2.8 Hz, 1H), 5.14 (dd, J=9.2, 2.9 Hz, 1H), 4.39-4.28 (m, 1H), 4.27-4.05 (m, 5H), 3.33-3.25 (m, 4H), 3.05-2.76 (m, 4H), 2.39-2.24 (m, 4H), 2.05-1.90 (m, 2H), 1.39 (s, 9H).

### General Procedure D (Fluoromethylation, Quaternization)

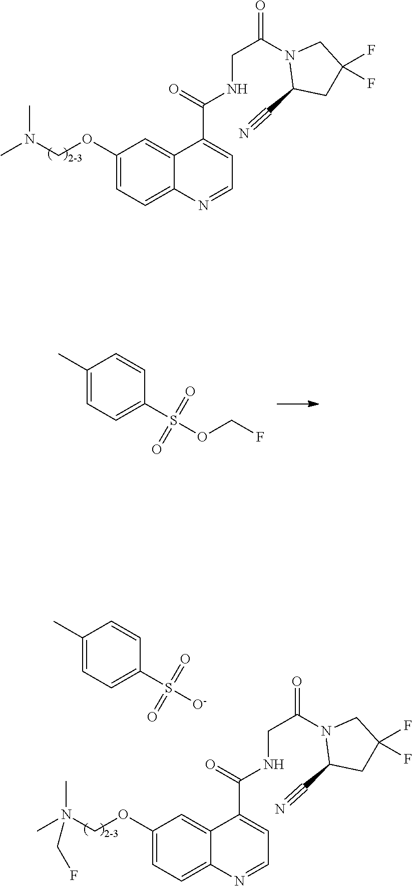

The required secondary-amine-containing UAMC1110 derivative (1 eq) was dissolved in DMF (reaction molarity≈0.4 M) in a glass vial. Fluoromethyl 4-methylbenzenesulfonate (5 eq) and DIPEA (5 eq) were added and the reaction was stirred at 120° C. for 20 min. The solvent was removed under reduced pressure and the crude mixture was purified via preparative RP-HPLC with a gradient of acetonitrile in water+0.1% formic acid (5-50% in 20′).

### General Procedure E (Fluoroethylation, Quaternization)

The required secondary-amine-containing UAMC1110 derivative (1 eq) was dissolved in acetonitrile (reaction molarity≈0.4 M) in a glass vial. Fluoroethyl 4-methylbenzenesulfonate (5 eq), DIPEA (5 eq) and NaI (excess) were added and the reaction was stirred at 80° C. for 120 min. The solvent was removed under reduced pressure and the crude mixture was purified via preparative RP-HPLC with a gradient of acetonitrile in water+0.1% formic acid (5-50%) in 20′.

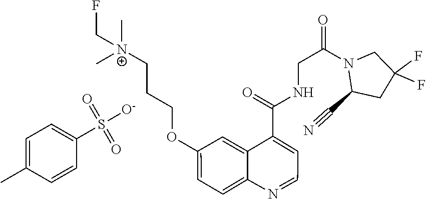 [SMILES: Cc1ccc(S(=O)(=O)[O-])cc1 ; C[N+](C)(CF)CCCOc1ccc2nccc(C(=O)NCC(=O)N3CC(F)(F)C[C@H]3C#N)c2c1]

(S)-3-((4-((2-(2-cyano-4,4-difluoropyrrolidin-1-yl)-2-oxoethyl)carbamoyl)quinoline-6-yl)oxy)-N-(fluoromethyl)-N,N-dimethylpropan-1-aminium benzenesulfonate (4a) (20 mg, 0.031 mmol, 47% yield) was prepared according to general procedure D with (S)—N-(2-(2-cyano-4,4-difluoropyrrolidin-1-yl)-2-oxoethyl)-6-(3-(dimethylamino)propoxy)quinoline-4-carboxamide (30 mg, 0.067 mmol). MS (ESI): m/z=239.7 [M+H]²⁺; 478.3 [M]⁺. ¹H NMR (400 MHz, MeOD) δ 8.77 (d, J=4.4 Hz, 1H), 8.04 (d, J=2.7 Hz, 1H), 7.99 (d, J=9.3 Hz, 1H), 7.68 (d, J=8.1 Hz, 2H), 7.57 (d, J=4.4 Hz, 1H), 7.47 (dd, J=9.2, 2.8 Hz, 1H), 7.21 (d, J=7.9 Hz, 2H), 5.46 (d, J=13.7 Hz, 2H), 5.15 (dd, J=9.4, 3.3 Hz, 1H), 4.37 (t, J=5.9 Hz, 2H), 4.32 (s, 2H), 4.25 (m, 1H), 4.12 (m, 1H), 3.70 (dd, J=10.6, 6.1 Hz, 2H), 3.22 (m, 6H), 2.89 (m, 2H), 2.41 (p, J=5.1 Hz, 2H), 2.33 (s, 3H). ¹³C NMR (101 MHz, MeOD) δ 169.2, 168.3, 157.32, 147.9, 144.0, 142.21, 141.6, 140.3, 129.7, 128.5, 126.0, 125.8, 125.5, 123.3, 119.9, 117.2, 104.3, 97.4, 95.2, 64.9, 59.3, 51.8, 51.5, 51.2, 46.6, 44.5, 44.5, 41.5, 36.8, 36.5, 36.3, 21.9, 19.9.

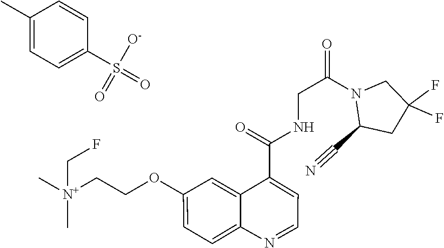 [SMILES: Cc1ccc(S(=O)(=O)[O-])cc1 ; C[N+](C)(CF)CCOc1ccc2nccc(C(=O)NCC(=O)N3CC(F)(F)C[C@H]3C#N)c2c1]

(S)-2-((4-((2-(2-cyano-4,4-difluoropyrrolidin-1-yl)-2-oxoethyl)carbamoyl)quinoline-6-yl)oxy)-N-(fluoromethyl)-N,N-dimethylethan-1-aminium 4-methylbenzenesulfonate (4b) was prepared according to general procedure D with (S)-2-((4-((2-(2-cyano-4,4-difluoropyrrolidin-1-yl)-2-oxoethyl)carbamoyl) quinoline-6-yl)oxy)-N-(fluoromethyl)-N,N-dimethylethan-1-aminium 4-methylbenzenesulfonate. MS (ESI): m/z=232.7 [M+H]²⁺; 464.2 [M]⁺. ¹H NMR (400 MHz, MeOD) δ 8.95 (s, 1H), 8.33 (d, J=2.7 Hz, 1H), 8.11 (d, J=9.3 Hz, 1H), 7.81 (d, J=4.9 Hz, 1H), 7.72-7.67 (m, 1H), 5.16 (dd, J=9.5, 3.3 Hz, 1H), 4.36 (s, 2H), 4.30-4.19 (m, 1H), 4.11 (dt, J=20.2, 10.3 Hz, 1H), 4.02-3.93 (m, 2H), 3.29 (d, J=2.1 Hz, 9H), 3.01-2.72 (m, 3H), 1.40-1.26 (m, 2H). ¹³C NMR (101 MHz, MeOD) δ 160.6, 159.9, 149.5, 137.8, 137.2, 134.2, 132.4, 120.5, 118.7, 117.6, 111.6, 109.3, 100.3, 97.4, 56.9, 54.7, 45.5, 43.5 (d, J=32.3 Hz), 43.3, 36.7 (d J=4.2 Hz), 33.7, 28.6.

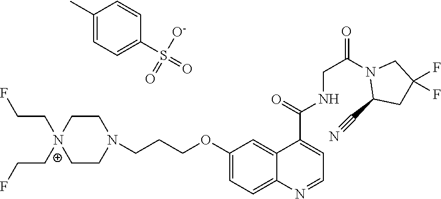

(S)-1-(4-(3-((4-((2-(2-cyano-4,4-difluoropyrrolidin-1-yl)-2-oxoethyl)carbamoyl)quinolin-6-yl)oxy)propyl)-1-(2-fluoroethyl)-1|4-piperazin-1-yl)-2-fluoroethan-1-ylium 4-methylbenzenesulfonate (4c) was prepared according to the general procedure E with (S)—N-(2-(2-cyano-4,4-difluoropyrrolidin-1-yl)-2-oxoethyl)-6-(3-(piperazin-1-yl)propoxy)quinoline-4-carboxamide. MS (ESI): m/z=375.6 [M+H]²⁺; 750.3 [M]⁺.

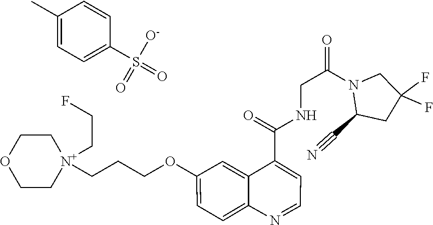 [SMILES: N#C[C@@H]1CC(F)(F)CN1C(=O)CNC(=O)c1ccnc2ccc(OCCC[N+]3(CCF)CCOCC3)cc12 ; Cc1ccc(S(=O)(=O)[O-])cc1]

(S)-4-(3-((4-((2-(2-cyano-4,4-difluoropyrrolidin-1-yl)-2-oxoethyl)carbamoyl)quinolin-6-yl)oxy)propyl)-4-(2-fluoroethyl)morpholin-4-ium 4-methylbenzenesulfonate (5) was prepared according to the general procedure E with (S)—N-(2-(2-cyano-4,4-difluoropyrrolidin-1-yl)-2-oxoethyl)-6-(3-morpholinopropoxy)quinoline-4-carboxamide. MS (ESI): m/z=267.6 [M+H]²⁺; 534.0 [M]⁺.

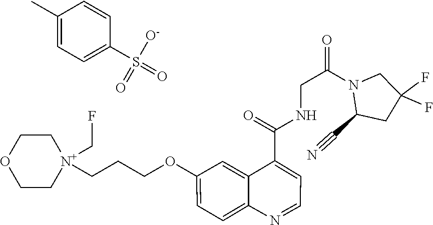 [SMILES: N#C[C@@H]1CC(F)(F)CN1C(=O)CNC(=O)c1ccnc2ccc(OCCC[N+]3(CF)CCOCC3)cc12 ; Cc1ccc(S(=O)(=O)[O-])cc1]

(S)-4-(3-((4-((2-(2-cyano-4,4-difluoropyrrolidin-1-yl)-2-oxoethyl)carbamoyl)quinolin-6-yl)oxy)propyl)-4-(2-fluoromethyl)morpholin-4-ium 4-methylbenzenesulfonate (6) was prepared according to the general procedure E with (S)—N-(2-(2-cyano-4,4-difluoropyrrolidin-1-yl)-2-oxoethyl)-6-(3-morpholinopropoxy)quinoline-4-carboxamide. MS (ESI): m/z=520.3 [M]⁺.

General Procedure F (Nucleophilic Substitution with Linker)

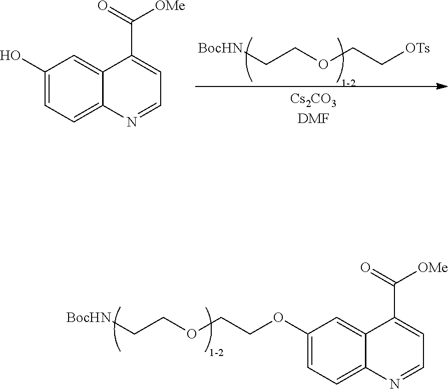

Methyl 6-hydroxyquinoline-4-carboxylate (200 mg, 0.98 mmol, 1.0 eq) was stirred under argon atmosphere at 0° C. with Cs₂CO₃ (639 mg, 1.96 mmol, 2.0 eq) in dry DMF (reaction molarity≈0.2 M) for 10 min. The alkylating agent (1.08 mmol, 1.1 eq) was added and the mixture was stirred at 40° C. overnight. The reaction mixture was poured on ice-cold water and extracted (3×20 mL) with EtOAc. The combined organic phases were washed with H₂O (20 mL), dried over Na₂SO₄ and the solvent was removed under reduced pressure. The title compound was purified using column chromatography eluting with a gradient of MeOH in DCM.

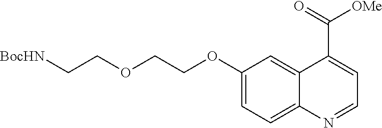 [SMILES: COC(=O)c1ccnc2ccc(OCCOCCNC(=O)OC(C)(C)C)cc12]

Methyl 6-(2-(2-((tert-butoxycarbonyl)amino)ethoxy)ethoxy)quinoline-4-carboxylate (7a) (450 mg, 1.15 mmol, 90% yield) was prepared according to general procedure F with 2-(2-((tert-butoxycarbonyl)amino)ethoxy)ethyl 4-methylbenzenesulfonate. MS (ESI): m/z=391.2 [M+H]⁺. ¹H NMR (400 MHz, DMSO) δ 8.89 (d, J=4.5 Hz, 1H), 8.09-8.01 (m, 2H), 7.93 (d, J=4.4 Hz, 1H), 7.53 (dd, J=9.2, 2.8 Hz, 1H), 6.84 (t, J=5.8 Hz, 1H), 4.24 (t, J=4.4 Hz, 2H), 3.98 (s, 3H), 3.84-3.79 (m, 2H), 3.48 (t, J=6.0 Hz, 2H), 3.11 (q, J=6.0 Hz, 2H), 1.35 (s, 9H). ¹³C NMR (101 MHz, DMSO) δ 166.3, 157.8, 155.6, 147.5, 144.8, 132.61, 131.3, 125.6, 122.6, 122.4, 104.9, 77.6, 69.3 (2C), 68.4, 67.6, 52.8, 28.2 (3C).

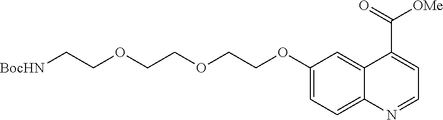 [SMILES: COC(=O)c1ccnc2ccc(OCCOCCOCCNC(=O)OC(C)(C)C)cc12]

Methyl 6-((2,2-dimethyl-4-oxo-3,8,11-trioxa-5-azatridecan-13-yl)oxy)quinoline-4-carboxylate (7b) (408 mg, 0.94 mmol, 96% yield) was prepared according to general procedure F with 2,2-dimethyl-4-oxo-3,8,11-trioxa-5-azatridecan-13-yl 4-methylbenzenesulfonate. MS (ESI): m/z=435.3 [M+H]⁺. ¹H NMR (400 MHz, CDCl₃) δ 8.85 (d, J=4.5 Hz, 1H), 8.24 (d, J=2.8 Hz, 1H), 8.05 (d, J=9.2 Hz, 1H), 7.91 (d, J=4.5 Hz, 1H), 7.46 (dd, J=9.2, 2.8 Hz, 1H), 5.04 (s, 1H), 4.31 (dd, J=5.7, 3.8 Hz, 2H), 4.01 (s, 3H), 3.97-3.92 (m, 2H), 3.74 (dd, J=5.8, 3.4 Hz, 2H), 3.66 (dd, J=5.8, 3.3 Hz, 2H), 3.55 (t, J=5.1 Hz, 2H), 3.32 (q, J=5.4 Hz, 2H), 1.41 (s, 9H). ¹³C NMR (101 MHz, CDCl₃) δ 166.8, 158.6, 156.1, 146.9, 145.5, 132.7, 131.4, 126.8, 123.3, 122.9, 104.1, 79.3, 70.9, 70.4 (2C), 69.7, 67.8, 52.8, 40.5, 28.5 (3C).

### General Procedure G

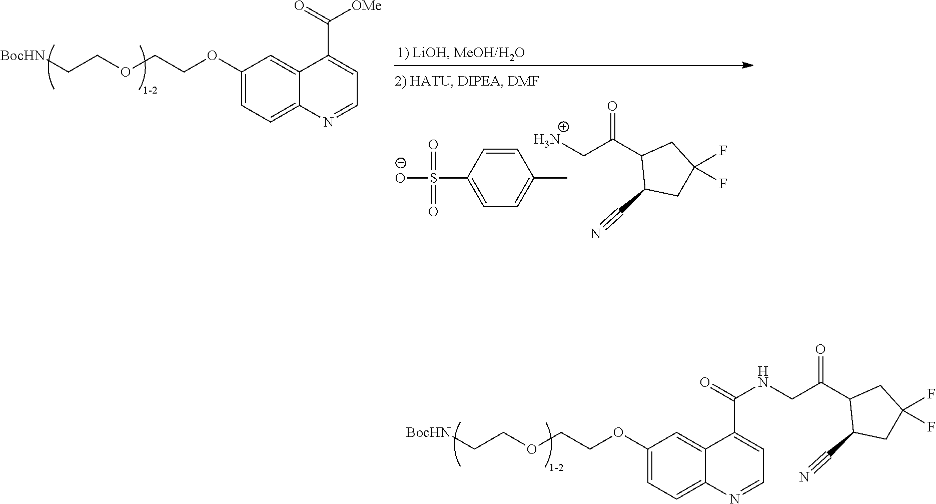

Corresponding compound 7 (0.50 mmol, 1.0 eq) was dissolved in MeOH/H₂O mixture and then LiOH (48 mg, 2.00 mmol, 4.0 eq) was added to the reaction mixture. After the consumption of the starting material (UPLC monitoring) MeOH was evaporated vacuo and the reaction mixture was carefully acidified to pH=4-5 by addition of 1M HCl at 0° C. Then the corresponding carboxylic acid was extracted (3×20 mL) with EtOAc, combined organic phases were dried over Na₂SO₄ and the solvent was evaporated via rotary evaporator. The carboxylic acid was dissolved in dry DMF and HATU (285 mg, 0.75 mmol, 1.5 eq) and dry DIPEA (0.26 mL, 1.5 mmol, 3.0 eq) were added to the reaction mixture. After 5 minutes stirring at room temperature, (S)-2-(2-cyano-4,4-difluoropyrrolidin-1-yl)-2-oxoethan-1-aminium 4-methylbenzenesulfonate (199 mg, 0.55 mmol, 1.1 eq) was added. After the consumption of the starting materials (UPLC monitoring) all volatiles removed via rotary evaporator. The crude was diluted with 25 mL of EtOAc, washed with saturated aqueous sodium bicarbonate and H₂O, dried over Na₂SO₄, filtered and the solvent was removed in vacuo to give a residue that was purified by reverse-phase column chromatography eluting with a gradient of ACN in water.

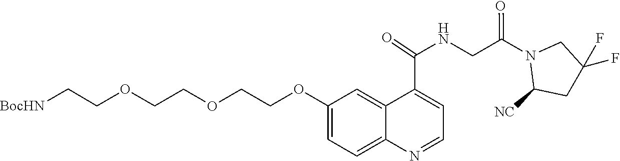 [SMILES: CC(C)(C)OC(=O)NCCOCCOCCOc1ccc2nccc(C(=O)NCC(=O)N3CC(F)(F)C[C@H]3C#N)c2c1]

tert-butyl (S)-(2-(2-(2-((4-((2-(2-cyano-4,4-difluoropyrrolidin-1-yl)-2-oxoethyl)carbamoyl)quinolin-6-yl)oxy)ethoxy)ethoxy)ethyl)carbamate (8a) (175 mg, 0.29 mmol, 59% yield) was prepared according to general procedure G with 6-((2,2-dimethyl-4-oxo-3,8,11-trioxa-5-azatridecan-13-yl)oxy)quinoline-4-carboxylic acid (206 mg, 0.49 mmol). MS (ESI): m/z=592.3 [M+H]⁺; 630.2 [M+K]⁺. ¹H NMR (400 MHz, MeOD) δ 8.70 (d, J=4.4 Hz, 1H), 7.96-7.87 (m, 2H), 7.52 (d, J=4.5 Hz, 1H), 7.45 (dd, J=9.3, 2.7 Hz, 1H), 5.13 (dd, J=9.3, 3.2 Hz, 1H), 4.38-4.18 (m, 5H), 4.18-4.05 (m, 1H), 3.92 (dd, J=5.9, 3.2 Hz, 2H), 3.76-3.68 (m, 2H), 3.66-3.59 (m, 2H), 3.50 (t, J=5.6 Hz, 2H), 3.21 (q, J=5.2 Hz, 2H), 2.99-2.75 (m, 2H), 1.40 (s, 9H). ¹³C NMR (101 MHz, MeOD) δ 170.4, 169.4, 159.1, 158.3, 148.1, 145.2, 142.5, 130.9, 127.5 (dd, J=250.8, 247.2 Hz), 127.1, 124.7, 120.4, 118.2, 105.21, 80.0, 71.6, 71.2, 71.0, 70.5, 69.2, 52.8 (t, J=32.3 Hz), 45.8 (d, J=5.6 Hz), 42.9, 41.2, 38.0 (t, J=25.1 Hz), 28.7 (3C).

### General Procedure H (Methylation, Quaternization)

The corresponding Boc-amine-containing derivative (0.10 mmol, 1.0 eq) was dissolved in acetonitrile and p-toluenesulfonic acid monohydrate (0.20 mmol, 2.0 eq) was added to the reaction mixture. After the consumption of the starting material (UPLC monitoring) the solvent was removed under reduced pressure. Then the residue was dissolved in DMF (reaction molarity≈0.4 M) in a glass vial. MeI (21 μL, 0.33 mmol, 3.3 eq) and DIPEA (10 eq) were added and the reaction was stirred at rt overnight. The solvent was removed under reduced pressure and the crude mixture was purified via preparative RP-HPLC with a gradient of ACN in Water+0.1% formic acid (5-50%) in 20′.

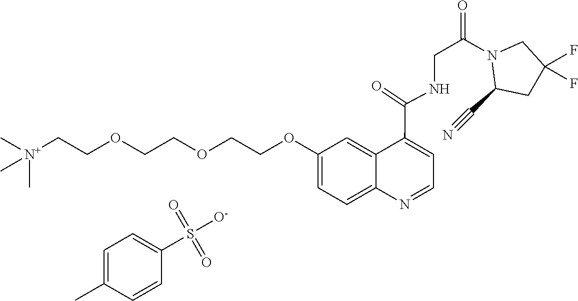 [SMILES: C[N+](C)(C)CCOCCOCCOc1ccc2nccc(C(=O)NCC(=O)N3CC(F)(F)C[C@H]3C#N)c2c1 ; Cc1ccc(S(=O)(=O)[O-])cc1]

(S)-2-(2-(2-((4-((2-(2-cyano-4,4-difluoropyrrolidin-1-yl)-2-oxoethyl)carbamoyl)quinolin-6-yl)oxy)ethoxy)ethoxy)-N,N,N-trimethylethan-1-aminium 4-methylbenzenesulfonate (7) (29 mg, 0.041 mmol, 41% yield) was prepared according to the general procedure H with tert-butyl (S)-(2-(2-(2-((4-((2-(2-cyano-4,4-difluoropyrrolidin-1-yl)-2-oxoethyl)carbamoyl)quinolin-6-yl)oxy)ethoxy)ethoxy)ethyl)carbamate. MS (ESI): m/z=267.6 [M+H]⁺⁺; 534.0 [M]⁺. ¹H NMR (400 MHz, MeOD) δ 8.77 (d, J=4.5 Hz, 1H), 8.04-7.94 (m, 2H), 7.70 (d, J=8.1 Hz, 2H), 7.58 (d, J=4.4 Hz, 1H), 7.51 (dd, J=9.2, 2.8 Hz, 1H), 7.24 (d, J=8.0 Hz, 2H), 5.15 (dd, J=9.4, 3.1 Hz, 1H), 4.41-4.21 (m, 5H), 4.21-4.06 (m, 1H), 3.93 (ddt, J=14.1, 4.9, 2.4 Hz, 4H), 3.78-3.65 (m, 4H), 3.58-3.48 (m, 2H), 3.14 (s, 9H), 3.03-2.74 (m, 2H), 2.37 (s, 3H). ¹³C NMR (101 MHz, MeOD) δ 170.6, 169.6, 159.2, 148.3, 145.3, 143.7, 142.8, 141.7, 131.1, 129.9 (2C), 127.5 (dd, J=250.9, 247.1 Hz), 127.3, 126.9 (2C), 124.5, 120.4, 118.4, 105.7, 71.5, 71.3, 70.5, 69.5, 66.9, 65.9, 54.8 (2C), 54.7, 52.9 (t, J=31.9 Hz), 45.84 (d, J=5.6 Hz), 42.9, 38.0 (t, J=25.2 Hz), 21.3.

### General Procedure I

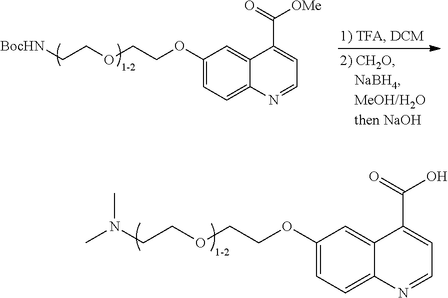

Trifluoroacetic acid (0.34 ml, 4.50 mmol, 5.0 eq) was added to the solution of the corresponding N-Boc protected methyl ester derivative (0.90 mmol, 1.0 eq) in dry DCM at 0° C. After stirring overnight at rt all volatiles were removed in vacuo and crude was dissolved in MeOH/H₂O mixture and cooled down to 0° C. Formaldehyde (37% solution in H₂O; 1.07 ml, 14.4 mmol, 16.0 eq) and NaBH₄ (170 mg, 4.5 mmol, 5.0 eq) were added to the reaction mixture. After stirring for 2 h at 0° C. the reaction mixture was alkalized to pH=12 and left at rt. After the consumption of the starting material (UPLC monitoring) all volatiles removed via rotary evaporator. The title compound was purified by reverse-phase column chromatography eluting with a gradient of ACN in water.

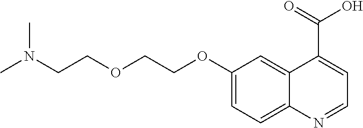 [SMILES: CN(C)CCOCCOc1ccc2nccc(C(=O)O)c2c1]

6-(2-(2-(Dimethylamino)ethoxy)ethoxy)quinoline-4-carboxylic acid (9a) (300 mg, 0.98 mmol, 52% yield) was prepared according to the general procedure I with methyl 6-(2-(2-((tert-butoxycarbonyl)amino)ethoxy)ethoxy)quinoline-4-carboxylate. MS (ESI): m/z=377.3 [M+H]⁺; 399.3 [M+Na]⁺. ¹H NMR (400 MHz, D₂O) δ 8.65 (d, J=4.5 Hz, 1H), 7.92 (d, J=9.2 Hz, 1H), 7.45 (ddd, J=12.1, 10.1, 2.6 Hz, 3H), 4.31-4.23 (m, 2H), 3.90-3.86 (m, 2H), 3.71-3.63 (m, 2H), 2.52 (t, J=5.7 Hz, 2H), 2.16 (s, 6H).

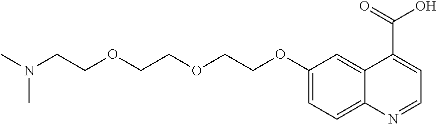 [SMILES: CN(C)CCOCCOCCOc1ccc2nccc(C(=O)O)c2c1]

6-(2-(2-(2-(dimethylamino)ethoxy)ethoxy)ethoxy)quinoline-4-carboxylic acid (9b) (207 mg, 0.59 mmol, 66% yield) was prepared according to the general procedure I with methyl 6-((2,2-dimethyl-4-oxo-3,8,11-trioxa-5-azatridecan-13-yl)oxy)quinoline-4-carboxylate. MS (ESI): m/z=349.2 [M+H]⁺; 371.2 [M+Na]⁺. ¹H NMR (400 MHz, MeOD) δ 8.66 (d, J=4.8 Hz, 1H), 7.96-7.83 (m, 2H), 7.57 (d, J=4.5 Hz, 1H), 7.41 (d, J=9.2 Hz, 1H), 4.25 (t, J=4.3 Hz, 2H), 3.87 (t, J=4.2 Hz, 2H), 3.69 (t, J=4.5 Hz, 2H), 3.65-3.48 (m, 4H), 2.46 (t, J=5.7 Hz, 2H), 2.20 (s, 6H). ¹³C NMR (101 MHz, MeOD) δ 174.7, 158.4, 148.4, 147.6, 145.4, 130.6, 127.6, 123.8, 120.4, 106.5, 71.6, 71.2, 70.6, 69.6, 68.8, 59.5, 49.9, 45.8.

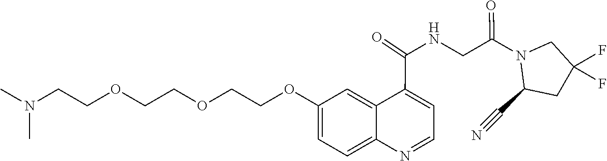 [SMILES: CN(C)CCOCCOCCOc1ccc2nccc(C(=O)NCC(=O)N3CC(F)(F)C[C@H]3C#N)c2c1]

(S)—N-(2-(2-cyano-4,4-difluoropyrrolidin-1-yl)-2-oxoethyl)-6-(2-(2-(2-(dimethylamino)ethoxy)ethoxy)ethoxy)quinoline-4-carboxamide (10a) (138 mg, 0.27 mmol, 41% yield) was prepared according to general procedure C with 6-(2-(2-(2-(dimethylamino)ethoxy)ethoxy)ethoxy)quinoline-4-carboxylic acid (226 mg, 0.65 mmol) and HATU. MS (ESI): m/z=260.8 [M+2H]⁺⁺; 520.3 [M+H]⁺. ¹H NMR (400 MHz, MeOD) δ 8.76 (d, J=4.4 Hz, 1H), 8.01-7.94 (m, 2H), 7.57 (d, J=4.4 Hz, 1H), 7.49 (dd, J=9.2, 2.8 Hz, 1H), 5.14 (dd, J=9.4, 3.2 Hz, 1H), 4.42-4.07 (m, 6H), 3.98-3.88 (m, 2H), 3.77-3.70 (m, 2H), 3.67-3.58 (m, 4H), 3.02-2.74 (m, 2H), 2.59 (td, J=5.7, 3.0 Hz, 2H), 2.30 (s, 6H). ¹³C NMR (101 MHz, MeOD) δ 170.7, 169.5, 159.4, 148.1, 145.4, 142.9, 130.9, 128.8 (t, J=250.7 Hz) 127.4, 124.9, 120.4, 118.2, 105.4, 71.7, 71.4, 70.6, 69.5, 69.4, 59.4, 52.9 (t, J=32.5 Hz), 45.8 (d, J=5.3 Hz), 45.6 (2C), 42.9, 38.1 (t, J=25.3 Hz).

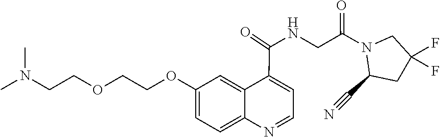 [SMILES: CN(C)CCOCCOc1ccc2nccc(C(=O)NCC(=O)N3CC(F)(F)C[C@H]3C#N)c2c1]

(S)—N-(2-(2-cyano-4,4-difluoropyrrolidin-1-yl)-2-oxoethyl)-6-(2-(2-(dimethylamino)ethoxy)ethoxy)quinoline-4-carboxamide (10b) (22 mg, 0.05 mmol, 8% yield) was prepared according to general procedure C with 6-(2-(2-(dimethylamino)ethoxy)ethoxy)quinoline-4-carboxylic acid (175 mg, 0.57 mmol) and COMU®. MS (ESI): m/z=476.3 [M+H]⁺. ¹H NMR (400 MHz, MeOD) δ 9.11 (t, J=5.8 Hz, 1H), 8.81 (d, J=4.2 Hz, 1H), 7.99 (d, J=9.2 Hz, 1H), 7.90 (d, J=2.3 Hz, 1H), 7.54-7.43 (m, 2H), 5.16 (d, J=7.1 Hz, 1H), 4.39-4.29 (m, 1H), 4.29-4.22 (m, J=6.1 Hz, 3H), 4.20-4.07 (m, 1H), 3.80 (s, 2H), 3.56 (t, J=5.8 Hz, 2H), 2.91-2.66 (m, 3H), 2.43 (t, J=5.9 Hz, 2H), 2.15 (s, 6H). ¹³C NMR (101 MHz, MeOD) δ 168.07, 167.48, 156.91, 147.67, 144.13, 140.85, 130.57, 125.33, 122.57, 119.06, 117.72, 104.54, 68.76, 68.51, 67.70, 67.68, 58.26, 45.57 (2C), 44.23, 41.37.

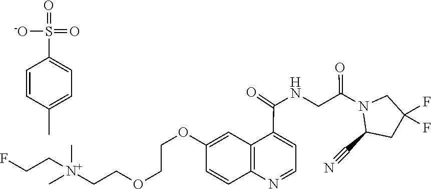

(S)-2-(2-((4-((2-(2-cyano-4,4-difluoropyrrolidin-1-yl)-2-oxoethyl)carbamoyl)quinolin-6-yl)oxy)ethoxy)-N-(2-fluoroethyl)-N,N-dimethylethan-1-aminium 4-methylbenzenesulfonate (8) (12 mg, 0.017 mmol, 41% yield) was prepared according to the general procedure E from compound 10b (20 mg, 0.04 mmol). MS (ESI): m/z=261.8 [M+2H]⁺⁺; 522.3 [M+H]⁺. ¹H NMR (400 MHz, MeOD) δ 8.75 (d, J=4.4 Hz, 1H), 8.01-7.95 (m, 2H), 7.67 (d, J=8.0 Hz, 1H), 7.55 (d, J=4.4 Hz, 1H), 7.46 (dd, J=9.3, 2.7 Hz, 1H), 7.20 (d, J=7.9 Hz, 1H), 5.10 (dd, J=9.4, 3.3 Hz, 1H), 4.41 (tt, J=7.6, 4.4 Hz, 2H), 4.31-4.19 (m, 3H), 4.12 (dd, J=19.5, 9.9 Hz, 1H), 4.00 (h, J=2.5 Hz, 2H), 3.94 (t, J=4.4 Hz, 2H), 3.88-3.83 (m, 1H), 3.80-3.76 (m, 1H), 3.74-3.68 (m, 2H), 3.19 (s, 6H), 3.01-2.72 (m, 2H). ¹³C NMR (101 MHz, MeOD) δ 170.7, 169.63, 159.2, 148.4, 145.7, 143.6, 142.9, 141.6, 131.1, 129.8, 127.5 (dd, J=251.1, 250.5 Hz), 126.9 (2C), 124.5, 120.4, 118.4, 105.8, 79.6, 77.9, 70.8, 69.0, 66.4 (d, J=19.8 Hz), 65.8, 65.7, 53.0 (t, J=32.2 Hz), 52.6, 45.9, 45.8, 42.9, 38.3, 37.9 (d, J=25.3 Hz), 21.3.

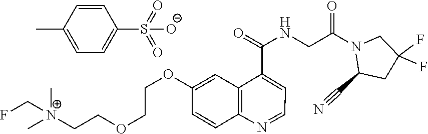 [SMILES: C[N+](C)(CF)CCOCCOc1ccc2nccc(C(=O)NCC(=O)N3CC(F)(F)C[C@H]3C#N)c2c1 ; Cc1ccc(S(=O)(=O)[O-])cc1]

(S)-2-(2-((4-((2-(2-cyano-4,4-difluoropyrrolidin-1-yl)-2-oxoethyl)carbamoyl)quinolin-6-yl)oxy)ethoxy)-N-(fluoromethyl)-N,N-dimethylethan-1-aminium 4-methylbenzenesulfonate (9) (23 mg, 0.034 mmol, 34% yield) was prepared according to the general procedure D from compound 10b (52 mg, 0.11 mmol). MS (ESI): m/z=254.7 [M+2H]⁺⁺; 508.2 [M+H]⁺. ¹H NMR (400 MHz, MeOD) δ 8.77 (d, J=4.4 Hz, 1H), 8.16-7.87 (m, 2H), 7.80-7.39 (m, 4H), 7.22 (d, J=7.6 Hz, 2H), 5.43 (d, J=45.5 Hz, 2H), 5.22-5.01 (m, 1H), 4.59-3.88 (m, 10H), 3.82-3.63 (m, 2H), 3.21 (s, 6H), 3.04-2.71 (m, 2H), 2.35 (s, 3H). ¹³C NMR (101 MHz, MeOD) δ 170.7, 169.6, 159.1, 148.4, 145.4, 143.6, 142.9, 141.7, 131.1, 129.9 (2C), 127.5 (dd, J=251.1, 250.5 Hz), 127.3, 126.9 (2C), 124.6, 120.4, 118.4, 105.8, 98.35 (d, J=218.3 Hz), 70.8, 69.0, 65.3 (2C), 62.5 (2C), 52.92 (t, J=32.2 Hz), 45.9 (d, J=4.9 Hz), 42.9, 38.0 (t, J=25.4 Hz), 21.3.

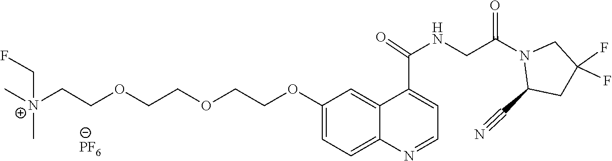

(S)-2-(2-(2-((4-((2-(2-cyano-4,4-difluoropyrrolidin-1-yl)-2-oxoethyl)carbamoyl)quinolin-6-yl)oxy)ethoxy)ethoxy)-N-(fluoromethyl)-N,N-dimethylethan-1-aminium hexafluorophosphate(V) (10) (12 mg, 0.017 mmol, 17% yield) was prepared according to the general procedure D from compound 10a (52 mg, 0.1 mmol). MS (ESI): m/z=276.7 [M+2H]⁺⁺; 552.3 [M+H]⁺. ¹H NMR (400 MHz, MeOD) δ 8.77 (d, J=4.4 Hz, 1H), 8.03-7.95 (m, 2H), 7.57 (d, J=4.5 Hz, 1H), 7.50 (dd, J=9.3, 2.8 Hz, 1H), 5.43 (d, J=45.2 Hz, 1H), 5.16-5.10 (m, 1H), 4.44-4.20 (m, 5H), 4.13 (dt, J=20.3, 10.3 Hz, 1H), 3.99-3.90 (m, 4H), 3.79-3.68 (m, 4H), 3.68-3.61 (m, 2H), 3.19 (d, J=2.1 Hz, 6H), 3.01-2.74 (m, 2H). ¹³C NMR (101 MHz, MeOD) δ 170.7, 169.6, 159.3, 148.3, 145.3, 142.9, 131.1, 127.5 (dd, J=251.1, 250.5 Hz), 127.3, 124.6, 120.4, 118.3, 105.7, 98.36 (d, J=219.2 Hz), 71.4 (2C), 70.6, 69.5, 65.3 (2C), 62.5 (2C), 52.9 (t, J=32.2 Hz), 45.9 (d, J=5.6 Hz), 42.9, 38.0 (t, J=25.2 Hz).

### General Procedure J (Methylation of Boc Piperazine)

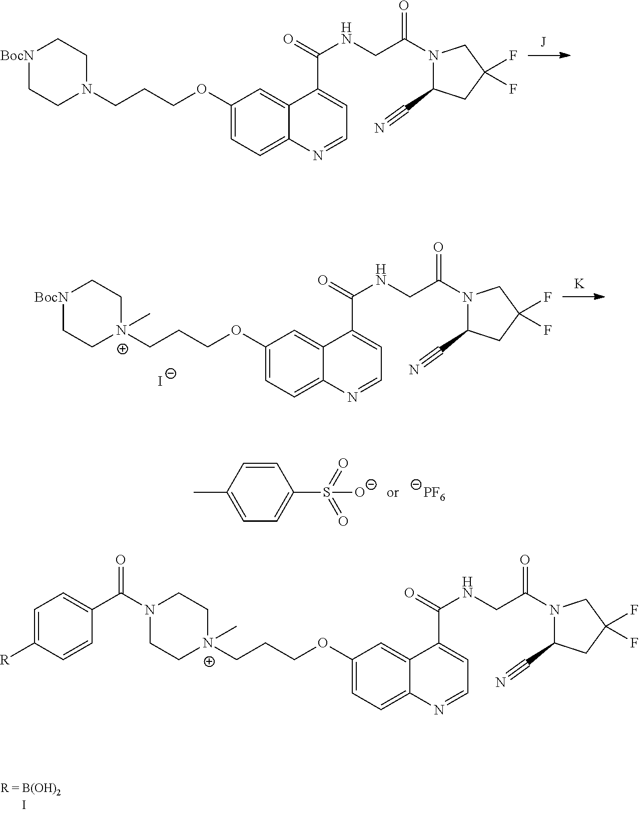

The corresponding Boc piperazine-containing derivative (0.35 mmol, 1.0 eq) was dissolved in DMF (reaction molarity≈0.4 M) in a glass vial. MeI (24 μL, 0.385 mmol, 1.1 eq) and DIPEA (0.12 mL, 0.70 mmol, 2.0 eq) were added and the reaction was stirred at rt overnight. The solvent was removed under reduced pressure and the crude mixture was purified via preparative RP-HPLC with a gradient of ACN in Water+0.1% formic acid (5-50%) in 20′.

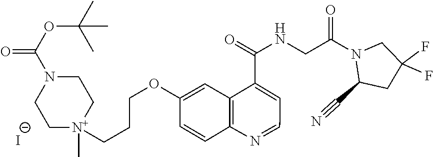 [SMILES: CC(C)(C)OC(=O)N1CC[N+](C)(CCCOc2ccc3nccc(C(=O)NCC(=O)N4CC(F)(F)C[C@H]4C#N)c3c2)CC1 ; [I-]]

(S)-4-(tert-butoxycarbonyl)-1-(3-((4-((2-(2-cyano-4,4-difluoropyrrolidin-1-yl)-2-oxoethyl)carbamoyl)quinolin-6-yl)oxy)propyl)-1-methylpiperazin-1-ium iodide (11) (127.5 mg, 0.175 mmol, 50% yield) was prepared according to the general procedure J from compound 3d (258 mg, 0.35 mmol). MS (ESI): m/z=301.3 [M+H]⁺⁺; 601.3 [M]⁺. ¹H NMR (400 MHz, MeOD) δ 8.78 (d, J=4.4 Hz, 1H), 8.06 (d, J=2.7 Hz, 1H), 8.00 (d, J=9.3 Hz, 1H), 7.57 (d, J=4.4 Hz, 1H), 7.52-7.48 (m, 1H), 5.14 (dd, J=9.4, 3.3 Hz, 1H), 4.45-4.21 (m, 5H), 4.13 (dt, J=20.2, 10.3 Hz, 1H), 3.99-3.86 (m, 2H), 3.78-3.62 (m, 4H), 3.55-3.48 (m, 4H), 3.22 (s, 3H), 3.02-2.74 (m, 2H), 2.48-2.37 (m, 2H), 1.48 (s, 9H). ¹³C NMR (101 MHz, MeOD) δ 170.8, 169.7, 158.8, 155.5, 148.5, 145.4, 143.1, 131.1, 127.6 (dd, J=250.9, 247.1 Hz), 127.3, 124.8, 120.3, 118.6, 105.7, 82.6, 66.4, 63.0, 62.9, 60.8 (2C), 52.9 (t, J=32.3 Hz), 47.2 (2C), 45.9 (d, J=6.5 Hz), 42.9, 37.9 (t, J=25.6 Hz), 28.5 (3C), 22.8.

### General Procedure K (Removal of Boc Protection Group and Amide Coupling)

The (S)-4-(tert-butoxycarbonyl)-1-(3-((4-((2-(2-cyano-4,4-difluoropyrrolidin-1-yl)-2-oxoethyl)carbamoyl)quinolin-6-yl)oxy)propyl)-1-methylpiperazin-1-ium iodide (51 mg, 0.07 mmol, 1.0 eq) was dissolved in dry MeCN, cooled down to 0° C. and p-toluenesulfonic acid monohydrate (53.2 mg, 0.28 mmol, 4.0 eq) was added to the reaction mixture. After stirring 2 days at rt the solvent was concentrated in vacuo. The crude was dissolved in dry DMF and added to the 10 mL round bottom flask that was previously charged with the corresponding carboxylic acid (0.105 mmol, 1.5 eq), HATU (53.2 mg, 0.14 mmol, 2.0 eq), dry DIPEA (0.12 mL, 0.7 mmol, 10.0 eq) and dry DMF (reaction molarity≈0.15 M). After the consumption of the starting materials (UPLC monitoring) all volatiles removed via rotary evaporator. The title compound was purified via RP-HPLC with a gradient of ACN in Water+0.1% formic acid (5-50% in 20′).

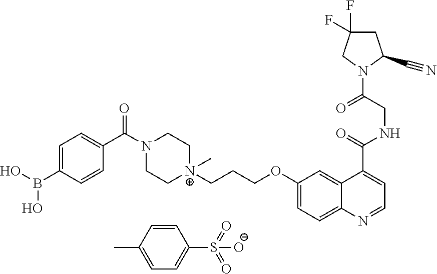 [SMILES: C[N+]1(CCCOc2ccc3nccc(C(=O)NCC(=O)N4CC(F)(F)C[C@H]4C#N)c3c2)CCN(C(=O)c2ccc(B(O)O)cc2)CC1 ; Cc1ccc(S(=O)(=O)[O-])cc1]

(S)-4-(4-boronobenzoyl)-1-(3-((4-((2-(2-cyano-4,4-difluoropyrrolidin-1-yl)-2-oxoethyl)carbamoyl)quinolin-6-yl)oxy)propyl)-1-methylpiperazin-1-ium 4-methylbenzenesulfonate (12) (32 mg, 0.039 mmol, 55% yield) was prepared according to general procedure K with 4-(4,4,5,5-tetramethyl-1,3,2-dioxaborolan-2-yl)benzoic acid (26 mg, 0.105 mmol, 1.5 eq). MS (ESI): m/z=325.3 [M+H]⁺⁺; 649.3 [M]⁺. ¹H NMR (400 MHz, MeOD) δ 8.78 (d, J=4.4 Hz, 1H), 8.05 (d, J=2.8 Hz, 1H), 8.00 (d, J=9.3 Hz, 1H), 7.87-7.71 (m, 2H), 7.69 (d, J=8.2 Hz, 2H), 7.57 (d, J=4.5 Hz, 1H), 7.50 (dd, J=9.3, 2.8 Hz, 1H), 7.45 (d, J=8.0 Hz, 2H), 7.22 (d, J=8.0 Hz, 2H), 5.13 (dd, J=9.3, 3.4 Hz, 1H), 4.41 (t, J=5.8 Hz, 2H), 4.36-4.19 (m, 3H), 4.13 (dt, J=20.2, 10.4 Hz, 1H), 4.04-3.44 (m, 10H), 3.27 (s, 3H), 3.02-2.75 (m, 2H), 2.48-2.38 (m, 2H), 2.35 (s, 3H). ¹³C NMR (101 MHz, MeOD) δ 172.7, 170.7, 169.7, 158.7, 148.5, 145.4, 143.6, 143.1, 141.7 (2C), 140.6, 139.4, 135.0, 131.1, 129.8 (2C), 127.5 (dd, J=250.9, 247.1 Hz), 127.3, 127.2 (2C), 126.9 (2C), 124.7, 120.3, 118.6, 105.8, 66.4, 63.2, 63.1, 60.8 (2C), 53.1 (t, J=31.9 Hz), 47.5 (2C), 46.0 (d, J=5.7 Hz), 42.9, 37.9 (t, J=25.9 Hz), 22.9, 21.3.

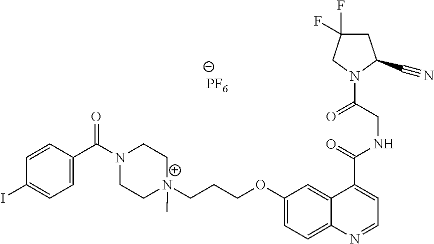 [SMILES: C[N+]1(CCCOc2ccc3nccc(C(=O)NCC(=O)N4CC(F)(F)C[C@H]4C#N)c3c2)CCN(C(=O)c2ccc(I)cc2)CC1 ; "PF6"]

(S)-1-(3-((4-((2-(2-cyano-4,4-difluoropyrrolidin-1-yl)-2-oxoethyl)carbamoyl)quinolin-6-yl)oxy)propyl)-4-(4-iodobenzoyl)-1-methylpiperazin-1-ium hexafluorophosphate(V) (13) (21.5 mg, 0.025 mmol, 35% yield) was prepared according to general procedure H with 4-iodobenzoic acid (26 mg, 0.105 mmol, 1.5 eq). MS (ESI): m/z=366.3 [M+H]⁺⁺; 731.3 [M]⁺. ¹H NMR (400 MHz, MeOD) δ 8.78 (d, J=4.5 Hz, 1H), 8.06 (d, J=2.8 Hz, 1H), 7.99 (d, J=9.3 Hz, 1H), 7.87 (d, J=8.4 Hz, 2H), 7.58 (d, J=4.5 Hz, 1H), 7.51 (dd, J=9.2, 2.8 Hz, 1H), 7.27 (d, J=8.4 Hz, 2H), 5.13 (dd, J=9.4, 3.3 Hz, 1H), 4.46-4.19 (m, 5H), 4.12 (dt, J=20.1, 10.2 Hz, 1H), 4.03-3.44 (m, 10H), 3.26 (s, 3H), 3.04-2.74 (m, 2H), 2.53-2.36 (m, 2H). ¹³C NMR (101 MHz, MeOD) δ 171.6, 170.6, 169.7, 158.8, 148.3, 145.1, 143.4, 139.2 (2C), 134.8, 130.8, 130.1 (2C), 127.5 (dd, J=251.1, 247.4 Hz), 127.3, 124.9, 120.3, 118.6, 105.7, 97.8, 66.5, 63.2, 63.1, 60.7 (2C), 52.9 (t, J=32.8 Hz), 47.5 (2C), 45.9 (d, J=5.1 Hz), 42.9, 37.9 (t, J=25.4 Hz), 22.9.

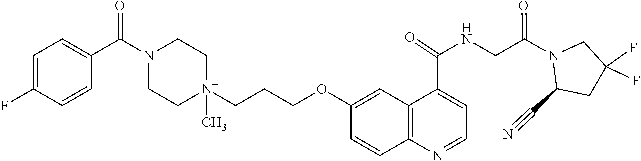 [SMILES: C[N+]1(CCCOc2ccc3nccc(C(=O)NCC(=O)N4CC(F)(F)C[C@H]4C#N)c3c2)CCN(C(=O)c2ccc(F)cc2)CC1]

(S)-1-(3-((4-((2-(2-cyano-4,4-difluoropyrrolidin-1-yl)-2-oxoethyl)carbamoyl)quinolin-6-yl)oxy)propyl)-4-(4-fluorobenzoyl)-1-methylpiperazin-1-ium (14) (10.2 mg, 0.016 mmol, 43% yield) was prepared according to general procedure H with 4-fluorobenzoic acid (7.4 mg, 0.05 mmol). MS (ESI): m/z=312.1 [M+H]⁺⁺; 623.1 [M]⁺. ¹H NMR (400 MHz, MeOD) δ 8.75 (d, J=4.4 Hz, 1H), 8.39 (s, 1H), 8.03-7.93 (m, 2H), 7.57-7.43 (m, 3H), 7.25-7.07 (m, 3H), 5.08 (dd, J=9.3, 3.5 Hz, 1H), 4.39 (t, J=5.8 Hz, 2H), 4.28 (d, J=5.2 Hz, 1H), 4.25-4.01 (m, 4H), 3.88 (s, 3H), 3.71 (s, 2H), 3.55 (s, 3H), 3.23 (s, 2H), 2.40 (s, 2H), 1.25 (s, 3H).

### 2) Biochemical Evaluation of Compounds

Typically, compounds are evaluated as inhibitors of FAP, PREP, DPP4, DPP8, DPP9 and DPP2.

Used Enzymes in these Assays:
A gateway-entry clone for human FAP was purchased from Dharmacon (Accession number DQ891423) and the human secretion signal was replaced with the HoneyBee mellitin secretion signal. For transfection and expression of FAP in Sf9 insect cells the C-terminal BaculoDirect kit from LifeTechnologies was used. The enzyme was purified from the supernatant of the insect cells using immobilized Ni-chelating chromatography (GE healthcare, Diegem, Belgium), followed by anion-exchange chromatography using a 1 mL HiTrap Q and size exclusion chromatography using the Superdex 200 column (GE healthcare, Diegem, Belgium).
Human recombinant PREP was expressed in BL21(DE3) cells and purified using immobilized Co-chelating chromatography (GE healthcare), followed by anion-exchange chromatography on a 1 ml Mono Q column (GE healthcare).
DPP4 was purified from human seminal plasma.
Gateway-entry clones for human DPP8 and DPP9 were purchased from Dharmacon (Accession numbers DQ891733 and DQ892325 respectively). For transfection and expression of DPP8 and DPP9 in Sf9 insect cells the N-terminal BaculoDirect kit from LifeTechnologies was used. The enzymes were purified using immobilized Ni-chelating chromatography (GE healthcare, Diegem, Belgium), followed by anion-exchange chromatography using a 1 mL Mono Q (GE healthcare, Diegem, Belgium).
Recombinant human DPP2 was purchased from R&D.

### IC₅₀ Measurements:

For FAP: IC₅₀ measurement of the probes was performed using Z-Gly-Pro-7-amino-4-methylcoumarine (AMC) (Bachem) as the substrate at a concentration of 50 μM at pH 8 (0.05 M Tris-HCl buffer, 1 mg/mL BSA and 140 mM NaCl). Eight concentrations of inhibitors/probe were tested. The final DMSO concentration was kept constant during the experiment to exclude any effects. Inhibitors were pre-incubated with the enzyme for 15 minutes at 37° C., afterwards the substrate was added and the velocities of AMC release were measured kinetically at λex=380 nm, λem=465 nm for at least 10 minutes at 37° C. Measurements were executed on the Infinite 200 (Tecan Group Ltd.) and the Magellan software was used to process the data.
For PREP:
   IC₅₀ measurements of the probes were carried out using N-succinyl-Gly-Pro-AMC (Bachem) as the substrate at a concentration of 250 μM at pH 7.4 (0.1 M K-phosphate, 1 mM EDTA, 1 mM DTT and 1 mg/mL BSA). Eight concentrations of inhibitors were tested. The final DMSO concentration in kept constant during the experiment to exclude any effects. Inhibitors were pre-incubated with the enzyme for 15 minutes at 37° C., afterwards the substrate was added and the velocities of AMC release were measured kinetically at λex=380 nm, λem=465 nm for at least 10 minutes at 37° C. Measurements were done on the Infinite 200 (Tecan Group Ltd.) and the Magellan software was used to process the data.
For DPP4, DPP8 & DPP9
   IC₅₀-values were determined using Ala-Pro-paranitroanilide (pNA) as the substrate at the respective final concentrations of 25 μM (DPP4), 300 μM (DPP8) or 150 μM (DPP9) at pH 7.4 (0.05 M HEPES-NaOH buffer with 0.1% Tween-20, 0.1 mg/mL BSA and 150 mM NaCl). At least eight different inhibitor concentrations were used. The final DMSO concentration is kept constant during the experiment to exclude any effects. Inhibitors were pre-incubated with the enzyme for 15 minutes at 37° C., afterwards the substrate was added and the velocities of pNA release were measured kinetically at 405 nm for at least 10 minutes at 37° C. Measurements were done on the Infinite 200 (Tecan Group Ltd.) and the Magellan software was used to process the data.
For DPP2: Initial velocity measurements were carried out. Lys-Ala-pNA was used as the substrate at a concentration of 1 mM at pH 5.5 (100 mM NaAc, 10 mM EDTA, 14 μg/mL aprotinin). Inhibitors were tested at one concentration, 5 μM, being the final concentration in the well. Inhibitors were pre-incubated with the enzyme for 15 minutes at 37° C., afterwards the substrate was added and the velocities of pNA release were measured kinetically at 405 nm for at least 10 minutes at 37° C. Measurements were done on the Infinite 200 (Tecan Group Ltd.) and the Magellan software was used to process the data.

### IC50 Results:

The specificity of the reference molecule UAMC1110 was tested by comparing the inhibitory effect of UAMC1110 on FAP, PREP, DPP2, DPP4, DPP8 and DPP9 activity. While UAMC1110 showed a high specificity to FAP (IC50=0.43±0.02 nM) compared to DPP2, DPP4, DPP8 and DPP9 (IC50>10 nM), UAMC1110 was shown to be an efficient PREP inhibitor as well (IC50=1.8±0.01 nM). Interestingly, a comparison between UAMC1110 and UAMC-0004522 (compound 4a) of current invention revealed that UAMC-0004522 has a similar IC50 for FAP (i.e. 0.32±0.02 nM), but a significantly higher IC50 for PREP (i.e. >10 nM) and for the other members of the prolyl oligopeptidase family S9 (Table 2). Similar results were obtained for all other tested compounds of the invention (Table 2), demonstrating that the compounds of current invention have a high specificity towards FAP which is currently not available in the art.

TABLE 2
Selectivity of the FAP-inhibitors for FAP, PREP, DPP4, DPP8 and DPP9 as indicated
by the IC50 values in nM for FAP and in μM for PREP, DPP4, DPP8, DPP9.

| | | | IC50 (μM) | | | |
|---|---|---|---|---|---|---|
| Cpd | UAMC- | FAP IC50 (nM) | PREP | DPP4 | DPP8 | DPP9 |
| 4a | 0004522 | 0.32 ± 0.02 | >10 | >10 | >10 | >10 |
| 4b | 0005086 | 2.53 ± 0.13 | >10 | >10 | >10 | >10 |
| 7 | 0005258 | 0.27 ± 0.02 | >10 | >10 | >10 | >10 |
| 8 | 0005095 | 9.42 ± 3.44 | >10 | >10 | >10 | >10 |
| 9 | 0005300 | 0.49 ± 0.001 | 5.328 ± 0.710 | / | / | / |
| 10 | 0005297 | 0.46 ± 0.04 | >1 | >1 | >10 | >10 |
| 11 | 0005269 | 0.44 ± 0.05 | >10 | >10 | >1 | 0.62 ± 0.08 |
| 12 | 0005288 | 0.57 ± 0.05 | >10 | >1 | >1 | >1 |
| 13 | 0005268 | 0.44 ± 0.1 | >10 | >10 | >1 | 0.67 ± 0.03 |
| 14 | 0005267 | 0.95 ± 0.1 | >10 | >10 | >10 | >1 |

Additionally the selectivity index was calculated. The selectivity index as used herein is the ratio of two SC-values. For example: the FAP-to-PREP selectivity index is defined as (IC₅₀(PREP)/IC₅₀(FAP)). In Table 3, the FAP-to-other enzyme selectivity indices are represented by the name of the other enzymes (PREP, DPP4, DPP8, DPP9). Hence, the selectivity index of PREP is IC₅₀(PREP)/IC₅₀(FAP). Compound 4b has thus a 4853 higher specificity for FAP than for PREP (Table 3).

TABLE 3
Selectivity indices of the FAP-inhibitors
towards FAP over PREP, DPP4, DPP8 and DPP9

Selectivity indices

| Compound | PREP | DPP4 | DPP8 | DPP9 |
|---|---|---|---|---|
| UAMC-0004522 | >31000 | >31000 | >31000 | >31000 |
| (4a) | | | | |
| UAMC-0005086 | >4800 | >4800 | >4800 | >4800 |
| (4b) | | | | |
| UAMC-0005258 | >36000 | >36000 | >36000 | >36000 |
| (7) | | | | |
| UAMC-0005095 | >1300 | >1300 | >1300 | >1300 |
| (8) | | | | |
| UAMC-0005300 | >10000 | >20000 | >20000 | >20000 |
| (9) | | | | |
| UAMC-0005297 | >2100 | >2100 | >21000 | >21000 |
| (10) | | | | |
| UAMC-0005269 | >22000 | >22000 | >2200 | >2200 |
| (11) | | | | |
| UAMC-0005288 | >17000 | >17000 | >1700 | >1700 |
| (12) | | | | |
| UAMC-0005268 | >22000 | >22000 | >2200 | >2200 |
| (13) | | | | |
| UAMC-0005267 | >10000 | >10000 | >10000 | >1000 |
| (14) | | | | |

### 3) Synthetic Approach, Procedures and Compound Characterization for the Radioactively Labeled Compound 1

The radiosynthesis was accomplished from the corresponding amine-based intermediate described in Scheme 1 (vide supra). The latter was subjected to reaction with dibromomethane-d2 in the presence of ¹⁸F-fluoride ion as shown in

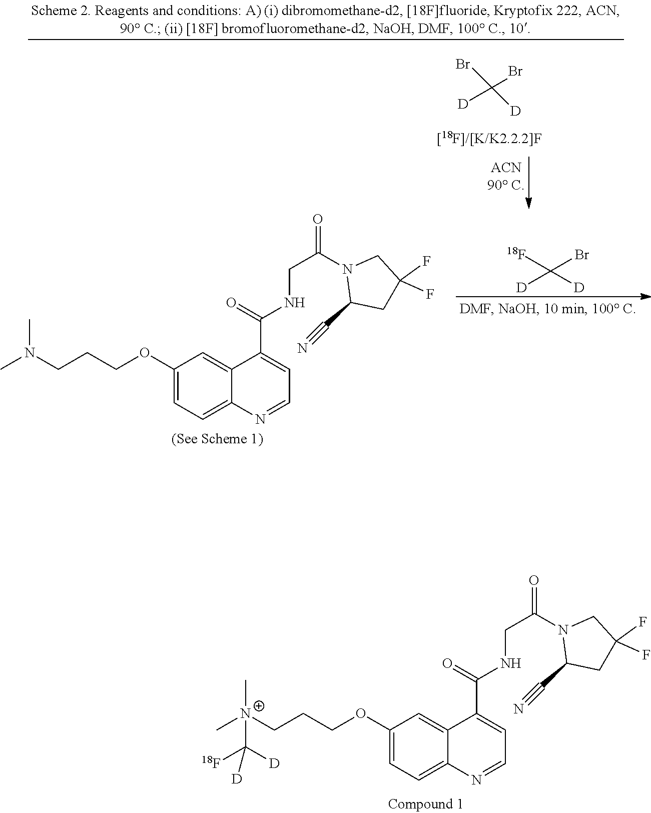 [SMILES: [2H]C([2H])(Br)Br ; CN(C)CCCOc1ccc2nccc(C(=O)NCC(=O)N3CC(F)(F)C[C@H]3C#N)c2c1 ; [2H]C([2H])([18F])Br ; [2H]C([2H])([18F])[N+](C)(C)CCCOc1ccc2nccc(C(=O)NCC(=O)N3CC(F)(F)C[C@H]3C#N)c2c1]

Procedure: The radiosynthesis was performed on a fully automated TRASIS AllinOne module (TRASIS). No-carrier added aqueous [¹⁸F]fluoride was produced in an Eclipse HP cyclotron (Siemens) using the ¹⁸O(p,n)¹⁸F reaction by proton bombardment of [¹⁸O]H₂O (Rotem Industries). After transferring to the hot cell, [¹⁸F]fluoride was isolated from enriched water by trapping in a silica-based anion exchange cartridge (QMA, Waters), previously conditioned with potassium bicarbonate solution (0.25 g/5 ml) and water (10 ml), followed by elution to reaction vial 1 with 0.8 mL of a mixture of 0.03 M K₂CO₃/0.07 M Kryptofix 222 in CH₃CN/H₂O (95:5 v/v) and evaporation to complete dryness. The thoroughly dried fluoride was cooled to 40° C. and dibromomethane-d2 (500 μL) in 500 μL ACN was added to reaction vial 1. The mixture was heated to 90° C. for 5 min. After cooling reactor 1 to 40° C., purification of [¹⁸F]FCD₂Br was performed by distillation over 4 silica plus SepPak® cartridges using a smooth He stream (1 min with 10 mL/min; then 40 mL/min for 10-15 min) and the pure [¹⁸F]FCD₂Br was trapped in reactor 2 and subsequently reacted with the amine precursor (2 mg in 200 μL DMF) in the presence of NaOH. The alkylation was performed for 10 min at 100° C. The reaction was cooled to RT and quenched by addition of 1 mL HPLC buffer (EtOH/0.05M NaOAc pH 5.5 15:85 (v/v)). The crude solution was purified by semi-preparative HPLC (Phenomenex Luna C18 250×10 mm (5 μm) HPLC column, EtOH/0.05M NaOAc pH 5.5 15:85 (v/v); 3 mL min⁻¹ flow rate). The fraction containing the desired molecule (t_{R}=14 min) was collected, loaded on a polymer-based weak cation exchange cartridge (Oasis WCX). Purified Compound 1 was obtained after successive washing steps with 6% NH₄OH, 6% NH₄OH/EtOH (50:50 v/v), EtOH, and H₂O. Compound 1 was eluted with a 0.9% NaCl solution.

Radiotracer characterization: To assess the quality and suitability of compound 1 ([¹⁸F]UAMC-4522) for specific applications, such as medical imaging, several parameters were determined. First, the decay-corrected RCY (Radiochemical Yield) was calculated. This indicates the percentage of the desired radioactively labeled molecule after correction for radioactive decay. It is calculated by dividing the amount of radioactively labeled molecule by the total radioactivity, taking into account the time elapsed since radiolabeling. Next, the Apparent Molar Activity (Am) was calculated. This represents the activity of the radiotracer per unit of substance (e.g. per micromole). It is calculated by dividing the activity of the radiotracer by the amount of labeled molecule used. Next, the Radiochemical Purity (RCP) was determined. This indicates the percentage of the radioactively labeled molecule relative to other chemical components or impurities. It is often determined using analytical techniques such as radio-HPLC (High-Performance Liquid Chromatography) or radio-TLC (Thin-Layer Chromatography). Finally, the Partition Coefficient (Log D) was calculated. This reflects the distribution of a substance between two solvents with different polarities. The Log D value represents the ratio of the concentrations of the substance in an organic solvent and an aqueous solution. It is used to understand the solubility and distribution of the radiotracer in biological systems.

Table 4 shows the characterization of compound 1. Data are presented as mean with standard deviation (n=8).

| | Decay- | | | |
|---|---|---|---|---|
| | corrected | Aₘ | | |
| Radiotracer | RCY (%)* | (GBq/μmol)* | RCP (%) | LogD |
| [¹⁸F]UAMC- | 2.6 ± 1.2 | 240.3 ± 171.3 | 99.2 ± 1.1 | −1.77 ± 0.01 |
| 4522 | | | | |

### 4) In Vivo Investigation of Compound 1 ([¹⁸F]UAMC-4522)

### Experimental Procedure

For all in vivo studies the following experimental procedure was used. Mice (7- to 8-week old female CD1−/− nude mice) were injected with 5.1-7.4 MBq of compound 1 (n=9) via the lateral tail vein. At 15, 30, 60 (n=3 for each time point) post-radiotracer injection (p.i.) the blood was collected in EDTA-coated tubes through cardiac puncture and the mice were euthanized by cervical dislocation.

### In Vivo Stability

From the collected blood, the plasma fraction was obtained by centrifugation at 4,000 g for 7 min, and mixed (200 μL) with an equal volume of cold MeCN to enable sample deproteination. After vigorously vortexing and γ-counting, the samples were centrifuged at 4000 g for 4 min, and the supernatant and the pellet were γ-counted separately to determine the amount of recovered radioactivity in the organic phase. The radioactive contents of the supernatant were analyzed by analytical radio-HPLC. The eluate fractions were collected every 30 s and counted for radioactivity in an automated γ-counter. Table 5 shows that the in vivo stability of compound 1 is significantly higher than that of the control.

TABLE 5
Radiotracer stability of [¹⁸F]UAMC-4522 (compound
1) and [⁶⁸Ga]Ga-DOTA-FAPI-04 (control) in CD1
nude mice after 15, 30, and 60 min p.i. (n =
3 per timepoint). Mean % intact radiotracer ± SD are described.

| Time (min) | UAMC-4522 | DOTA-FAPI-04 |
|---|---|---|
| 0 | 99.12 ± 0.51 | 97.56 ± 0.44 |
| 15 | 74.84 ± 6.62 | 22.82 ± 1.42 |
| 30 | 60.23 ± 3.60 | 14.49 ± 0.63 |
| 60 | 56.47 ± 7.81 | 13.99 ± 1.98 |

### Biodistribution of Compound 1 in Control Animals

The organs and tissues of the CD1−/− nude mice (see above) were harvested, weighed and the radioactivity in the samples was measured using an automatic γ-counter (Wizard² 2480, PerkinElmer). The uptake levels of the tracers were expressed as percentage of the injected dose per gram (% ID g⁻¹).

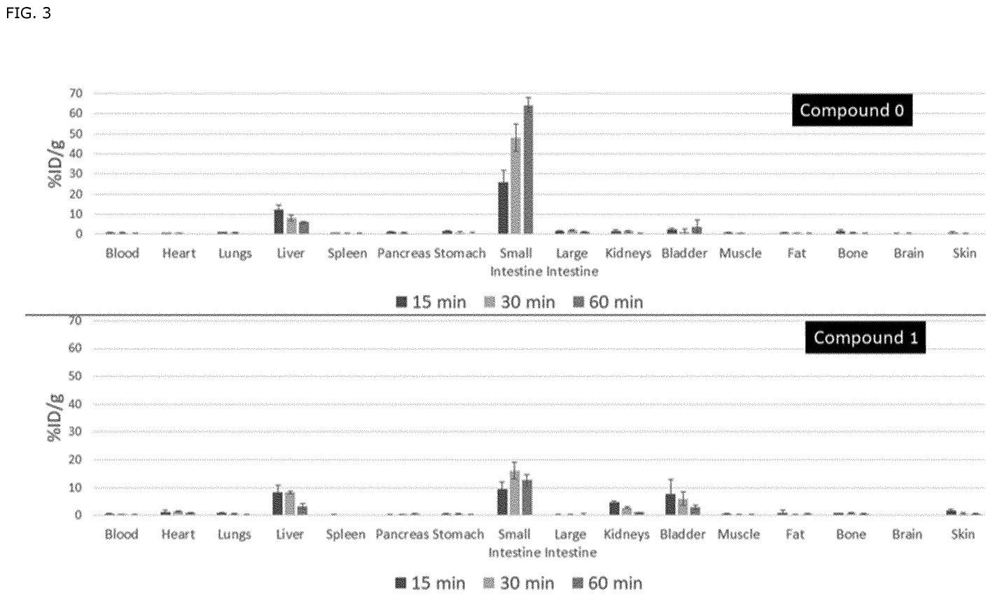

FIG. 3 summarizes the tissue biodistribution (% injected dose/gram of tissue) of [¹⁸F]UAMC-4522 (compound 1) and of compound 0 in normal CD1 nude mice after 15, 30, and 60 min p.i. (n=3 per timepoint).

### In Vivo PET Imaging and Biodistribution in Tumor-Bearing Animals

To confirm the binding specificity of compound 1, a cohort of tumor-bearing mice (n=4) was injected via the tail vein with UAMC1110 30 min before the injection of compound 1. Dynamic whole-body PET images were acquired during 60 min. The average tumor or muscle activity per volume was obtained from the PET images and the decay-corrected time-activity curves (TACs) were extracted. At the end of the scans, the organs and tissues were harvested, weighed and the radioactivity in the samples was measured using an automatic γ-counter.

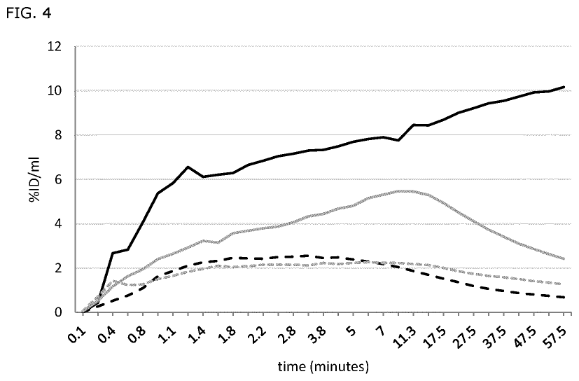

The radioligand's performance was compared with [68Ga]Ga-DOTA-FAPI-04 in U87MG xenografts (FIG. 4). Tumor uptake for both radioligands, represented as TACs, is shown in FIG. 4. Dynamic imaging over the course of 60 min p.i. for [18F]UAMC-4522 demonstrated an early peak uptake in the tumor followed by retention or slight increase in the tumor over the duration of the PET scan. [18F]UAMC-4522 had a remarkable and significantly higher tumor uptake compared to the uptake in the muscle (10.16±2.22 vs. 2.20±0.33% ID/mL at 60 min p.i.). In comparison, [68Ga]Ga-DOTA-FAPI-04 showed a later peak tumor uptake, reaching a maximum uptake at 8.8 min p.i. (5.46±0.83% ID/mL), followed by a fast clearance from the tumor (FIG. 4).

Importantly, the radiotracer's specificity for FAP was evaluated in a blocking study. Pre-injection of a specific FAP inhibitor, UAMC-1110, in mice bearing U87MG tumors led to a decrease in tumor uptake, demonstrating the specificity of the radioligand for FAP (FIG. 4). Interestingly, [18F]UAMC-4522 showed lower nonspecific tumor uptake when compared to the 68Ga-labeled FAP ligand, which can be an advantage when imaging sites with lower FAP expression.

In accordance with the PET results, the biodistribution study confirmed the high and specific tumor uptake of all FAP radioligands at 60 min post-injection (p.i.) (Table 6). The resulting tumor-to-background ratios are shown in Table 6.

TABLE 6
Tumor-to-background ratios of FAP radioligands in U87MG xenografts at 60 min p.i.

| | Tumor | Tumor-to- | Tumor-to- | Tumor-to-small | Tumor-to- |
|---|---|---|---|---|---|
| Radiotracer | (% ID/g) | muscle | blood | intestine | liver |
| UAMC-4522 | 10.98 ± 1.73 | 6.54 ± 1.36 | 6.35 ± 1.08 | 2.43 ± 0.12 | 4.77 ± 0.58 |
| DOTA-FAPI-04 | 3.67 ± 0.73 | 11.49 ± 3.81 | 5.06 ± 1.06 | 8.22 ± 1.94 | 1.87 ± 0.49 |
| Compound 0 | 3.02 ± 0.39 | 6.77 ± 3.79 | 6.99 ± 0.57 | 0.14 ± 0.02 | 1.32 ± 0.73 |

### Ex Vivo Tumor Analysis

Tumor sections (10 μm) were taken at regular intervals across the entire tumor volume and used for immunohistochemical analysis of FAP expression in the tumor xenografts. Tumor sections were stained for FAP and after image acquisition, FAP expression was quantified. For correlation analysis, the Pearson correlation coefficient was computed.

U87MG tumors showed a high FAP positive tumor area (75.9±11.6%). The uptake of [18F]UAMC-4522 (r=0.967, p=0.032) was highly correlated to FAP expression in the tumors. In contrast, animals injected with [68Ga]Ga-DOTA-FAPI-04 (r=0.892, p=0.108), showed a poorer correlation between mean tumor radioactivity and FAP expression in U87MG tumors. These results demonstrate that [18F]UAMC-4522 uptake in tumors is a better proxy for FAP activity and/or expression than [68Ga]Ga-DOTA-FAPI-04, potentially due to the lack of in vivo stability of the latter.

### DESCRIPTION OF FIGURES

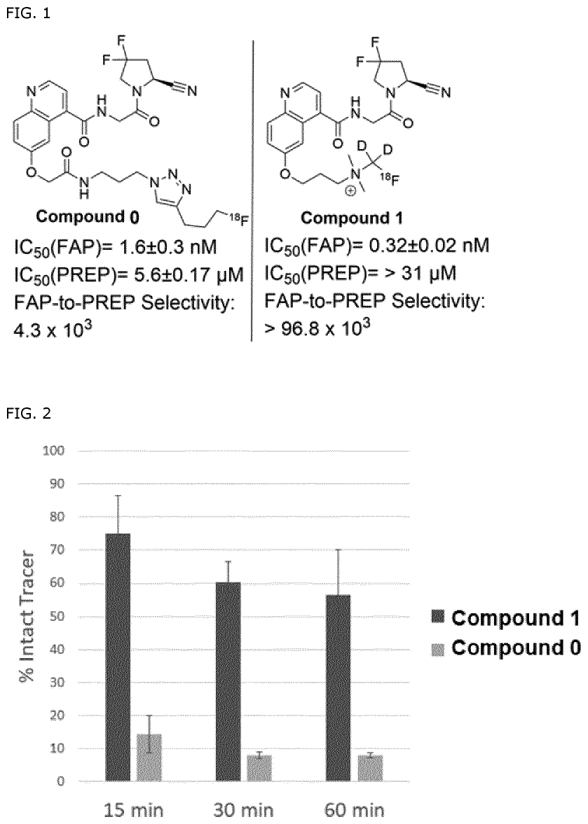

FIG. 1 shows a comparison of Compound 1 with a compound without quaternary ammonium cation (Compound 0) regarding their selectivity for FAP.

The importance of a quaternary ammonium group in the linker moiety is demonstrated by comparing their selectivity for FAP for Compounds 0 and 1. Only compound 1 has a quaternary ammonium-containing linker and this molecule has a significantly higher selectivity for FAP with respect to prolyl oligopeptidase (PREP) than Compound 0. PREP is an enzyme that is closely related to FAP and that is widely expressed in the human body. Selectivity for FAP with respect to PREP thus is a relevant parameter.

FIG. 2 shows a comparison of Compound 1 with a compound without quaternary ammonium cation (Compound 0) regarding their metabolic stability.

The importance of a quaternary ammonium group in the linker moiety is demonstrated by comparing their metabolic stability for Compounds 0 and 1. Compound 1 has a significantly higher metabolic stability than Compound 0. This is also relevant, because better metabolic stability can allow for less frequent dosing during in vivo applications.

FIG. 3 shows a comparison of Compound 1 with a compound without quaternary ammonium cation (Compound 0) regarding their biodistribution profile.

The importance of a quaternary ammonium group in the linker moiety is demonstrated by comparing their biodistribution profile for Compounds 0 and 1. The quaternary ammonium-comprising Compound 1, has a biodistribution profile including urinary excretion while Compound 0 is characterized by strong hepatobiliary secretion and almost exclusive excretion via the gut. The latter can be an undesirable feature, for example in the framework of radio-imaging and radiotherapy. More specifically, important gut excretion causes a strong background signal in diagnostic imaging applications. Likewise, it can impose a radio-toxicological burden on the gut and the rest of the body in radiotherapeutic applications.

FIG. 4 shows the in vivo uptake of [18F]UAMC-4522 (black) and [68Ga]Ga-DOTA-FAPI-04 (grey) in % ID/ml in tumor tissue over the course of 60 minutes post-injection with (dotted lines) or without (straight lines) a pre-injection of UAMC-1110.

The importance of a quaternary ammonium group in the linker moiety is demonstrated by comparing their uptake into tumor tissue in vivo. Compound 1 has a significantly higher uptake compared to FAPI-04, a reference radiotracer molecule lacking a quaternary ammonium.

1. A compound of Formula I or a stereoisomer, tautomer, racemic, metabolite, prodrug, salt, hydrate, or solvate thereof, wherein the compound comprises a quinoline structure, 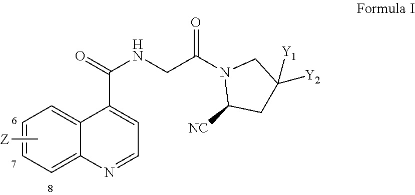
wherein
Y₁ and Y₂ are independently H or F; wherein a linker (Z) comprises an oxygen, wherein said oxygen is covalently bound to the quinoline structure of said compound on position 6, 7 or 8, wherein said linker comprises a quaternary ammonium cation.

2. The compound according to claim 1, wherein a radionuclide is covalently bound to the linker and wherein the radionuclide is selected from ¹⁸F, ¹²⁰I, ¹²²I, ¹²³I, ¹²⁴I, ¹²⁵I, ¹³¹I or ²¹¹At.

3. The compound according to claim 1, wherein said linker has a molecular weight of maximal 1000 Da.

4. The compound according to claim 1, wherein said linker (Z) is selected from 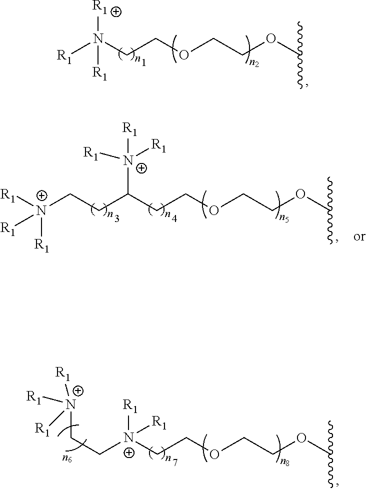
wherein each R₁ is independently selected from
−H, —CH₃, CH₂CH₃, —CH₂CH₂CH₃, —CH₂CH₂CH₂CH₃, —CH₂F, CH₂CH₂F, —CH₂CH₂CH₂F, —CH₂CH₂CH₂CH₂F, —CH₂I, CH₂CH₂I, —CH₂CH₂CH₂I, or —CH₂CH₂CH₂CH₂I, wherein F is present as ¹⁸F and I as ¹²⁰I, ¹²²I, ¹²³I, ¹²⁴I, ¹²⁵I, or ¹³¹I, and wherein n₁, n₂, n₃, n₄, n₅, n₆, n₇, n₈ is independently 0-4.

5. The compound according to claim 1, wherein said linker (Z) is selected from 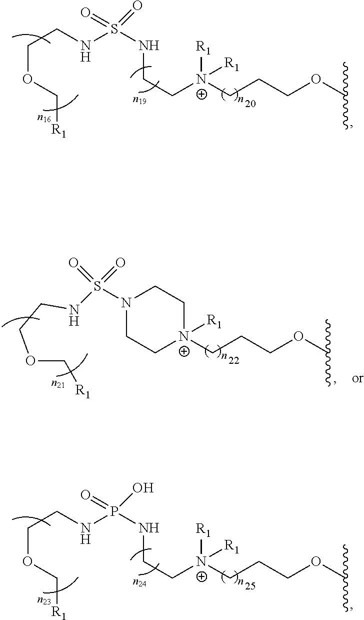
wherein each R₁ is independently selected from
—H, —CH₃, CH₂CH₃, —CH₂CH₂CH₃, —CH₂CH₂CH₂CH₃, —CH₂F, CH₂CH₂F, —CH₂CH₂CH₂F, —CH₂CH₂CH₂CH₂F, —CH₂I, CH₂CH₂I, —CH₂CH₂CH₂I, and —CH₂CH₂CH₂CH₂I, —COOCCH₃ or COC₆H₆—R₂, wherein R₂ is selected I, F, At or B(OH)₂, and wherein F is present as ¹⁸F and I as ¹²⁰I, ¹²²I, ¹²³I, ¹²⁴I, ¹²⁵I or ¹³¹I, At as ²¹¹At and wherein n₉, n₁₀, n₁₁, n₁₂, n₁₃, n₁₄, n₁₅, n₁₆, n₁₇ is independently 0-4.

6. The compound according to claim 1, wherein said linker (Z) is selected from 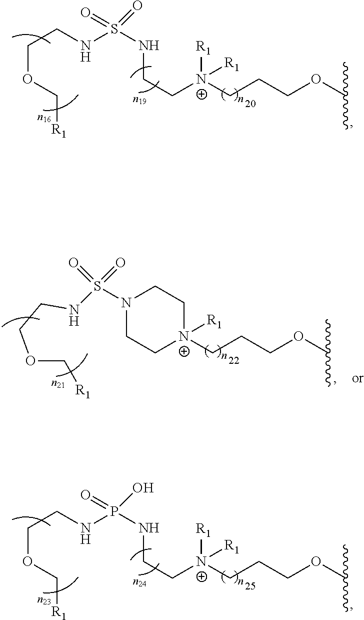
wherein each R₁ is independently selected from
—H, —CH₃, CH₂CH₃, —CH₂CH₂CH₃, —CH₂CH₂CH₂CH₃, —CH₂F, CH₂CH₂F, —CH₂CH₂CH₂F, —CH₂CH₂CH₂CH₂F, —CH₂I, CH₂CH₂I, —CH₂CH₂CH₂I, or —CH₂CH₂CH₂CH₂I, wherein F is present as ¹⁸F and I as ¹²⁰I, ¹²²I, ¹²³I, ¹²⁴I, ¹²⁵I or ¹³¹I, and wherein n₁₈, n₁₉, n₂₀, n₂₁, n₂₂, n₂₃, n₂₄, n₂₅ is independently 0-4.

7. The compound according to claim 1, wherein said linker (Z) comprises an aromatic ring, optionally heterocyclic and 5-, 6- or 7-membered.

8. The compound according to any of the previous claim 1, wherein said linker (Z) is selected from 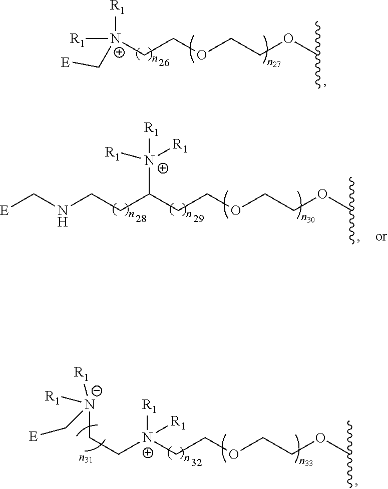
wherein
each R₁ is independently selected from
—H, —CH₃, CH₂CH₃, —CH₂CH₂CH₃, or —CH₂CH₂CH₂CH₃;
n₂₆, n₂₇, n₂₈, n₂₉, n₃₀, n₃₁, n₃₂, n₃₃ is independently 0-4;
E is 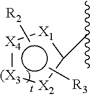
wherein each X₁, X₂, X₃, X₄ is independently selected from C, O or N;
t is independently 1, 2 or 3;
each R₂ is independently selected from
¹⁸F, ¹²⁰I, ¹²²I, ¹²³I, ¹²⁴I, ¹²⁵I or ¹³¹I or ²¹¹At; and
each R₃ is independently selected from
guanidine, aminomethyl or dialkylaminomethyl.

9. The compound according to claim 1, wherein said linker (Z) is selected from 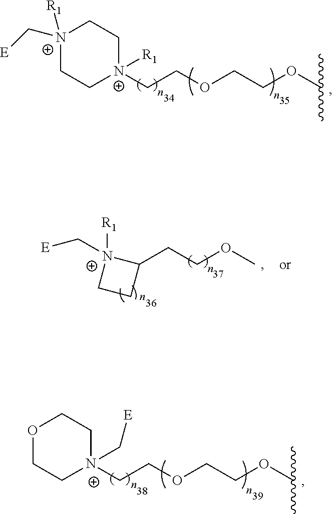
wherein
each R₁ is independently selected from
—H, —CH₃, CH₂CH₃, —CH₂CH₂CH₃, or —CH₂CH₂CH₂CH₃;
n₃₄, n₃₅, n₃₆, n₃₇, n₃₈, n₃₉ is independently 0-4;
E is 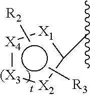
wherein each X₁, X₂, X₃, X₄ is independently selected from C, O or N;
t is independently 1, 2 or 3;
each R₂ is independently selected from
¹⁸F, ¹²⁰I, ¹²²I, ¹²³I, ¹²⁴I, ¹²⁵I or ¹³¹I or ²¹¹At; and
each R₃ is independently selected from
guanidine, aminomethyl or dialkylaminomethyl.

10. A pharmaceutical composition comprising a compound according to claim 1 and at least one pharmaceutically acceptable carrier, diluent, excipient, or adjuvant.

11.-15. (canceled)

16. A method for the prevention and/or treatment of a disorder, said method comprises administering to a subject in need thereof a compound according to claim 1.

17. A method for the prevention and/or treatment of a FAP-related disorder, said method comprises administering to a subject in need thereof a compound according to claim 1.

18. The method according to claim 17, wherein said FAP-related disorder is selected from proliferative diseases selected from breast cancer, colorectal cancer, ovarian cancer, prostate cancer, pancreatic cancer, kidney cancer, lung cancer, melanoma, fibrosarcoma, bone and connective tissue sarcomas, renal cell carcinoma, giant cell carcinoma, squamous cell carcinoma, and adenocarcinoma; diseases characterized by tissue remodeling and/or chronic inflammation such as fibrotic diseases, wound healing disorders, keloid formation disorders, osteoarthritis, rheumatoid arthritis, cartilage degradation disorders, atherosclerotic disease and Crohn's disease; disorders involving endocrinological dysfunction, such as disorders of glucose metabolism; or blood clotting disorders.

19. A method for tissue and/or organ imaging, said method comprises administering to a subject in need thereof a compound according to claim 1.

20. A method for a companion diagnostic, said method comprises administering to a subject in need thereof a compound according to claim 1.
